SEQUENCE LISTING (112 sequences)

SEQ ID NO: 1 - DNA, 1172 bp, Homo sapiens
gcgatgcgggcgcccccggcgggcggccccggcgggcaccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagggctgccactccccaccaccgtccccagccccgttctgttccgggctgggactctgcccccggagcaccctccccagctgacatcacccatcccactccagccccaggcccctctgcccacgctgcacccagcaccaccagcgccctgacccccggacctgccgctgccgctgccgacgccgcagcttcctccgttgccaagggcggggcttagagctcaacccagacacctgcaggtgccggaagctgcgaaggtgacacatggcttttcagactcagcagggtgacttgcctcagaggctatatcccagtgggggaacaaagaggagcctggtaaaaaacagccaagcccccaagacctcagcccaggcagaagctgctctaggacctgggcctctcagagggctcttctgccatcccttgtctccctgaggccatcatcaaacaggacagagttggaagaggagactgggaggcagcaagaggggtcacataccagctcaggggagaatggagtactgtctcagtttctaaccactctgtgcaagtaagcatcttacaactggctcttcctcccctcactaagaagacccaaacctctgcataatgggatttgggctttggtacaagaactgtgacccccaaccctgataaaagagatggaaggaaaaaaaaaaaaaaaaaaaaaaaaaaaaaaaaaaaaaaaaaaaaaaaaaaaaaaa

SEQ ID NO: 2 - protein, 207 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 3 - protein, 188 aa, Homo sapiens [sequence omitted]

SEQ ID NO: 4 - protein, 207 aa, Homo sapiens; identical to SEQ ID NO: 2

SEQ ID NO: 5 - DNA, 1236 bp, Mus musculus
ctcaggccgtcgctgcggcgctgcgttgcgctgcctgcgcccagggctcgggagggggccgcggaggagccgccccctgcgccccgccccgggtccccgggcccgcgccatggggctctggctgccgccgccccccacgccgccgggctagggccatgcgggcgctcccggcgctcgccccccgcgggcaccatgagccccctgctccgtcgcctgctgcttgttgcactgctgcagctggctcgcacccaggcccctgtgtcccagtttgatggccccagccaccagaagaaagtggtgccatggatagacgtttatgcacgtgccacatgccagcccagggaggtggtggtgcctctgagcatggaactcatgggcaatgtggtcaaacaactagtgcccagctgtgtgactgtgcagcgctgtggtggctgctgccctgacgatggcctggaatgtgtgcccactgggcaacaccaagtccgaatgcagatcctcatgatccagtacccgagcagtcagctgggggagatgtccctggaagaacacagccaatgtgaatgcagaccaaaaaaaaaggagagtgctgtgaagccagacagggttgccataccccaccaccgtccccagccccgctctgttccgggctgggactctaccccgggagcatcctccccagctgacatcatccatcccactccagccccaggatcctctgcccgccttgcacccagcgccgtcaacgccctgacccccggacctgccgctgccgctgcagacgccgccgcttcctccattgccaagggcggggcttagagctcaacccagacacctgtaggtgccggaagccgcgaaagtgacaagctgctttccagactccacgggcccggctgcttttatggccctgcttcacagggagaagagtggagcacaggcgaacctcctcagtctgggaggtcactgccccaggacctggaccttttagagagctctctcgccatcttttatctcccagagctgccatctaacaattgtcaaggaacctcatgtctcacctcaggggccagggtactctctcacttaaccaccctggtcaagtgagcatcttctggctggctgtctcccctcactatgaaaaccccaaacttctaccaataacgggatttgggttctgttatgataactgtgacacacacacacactcacactctgataaaagagatggaagacactaac

SEQ ID NO: 6 - protein, 207 aa, Mus musculus
MSPLLRRLLLVALLQLARTQAPVSQFDGPSHQKKVVPWIDVYARATCQPREVVVPLSMELMGNVVKQLVPSCVTVQRCGGCCPDDGLECVPTGQHQVRMQILMIQYPSSQLGEMSLEEHSQCECRPKKKESAVKPDRVAIPHHRPQPRSVPGWDSTPGASSPADIIHPTPAPGSSARLAPSAVNALTPGPAAAAADAAASSIAKGGA

SEQ ID NO: 7 - protein, 188 aa, Mus musculus
MSPLLRRLLLVALLQLARTQAPVSQFDGPSHQKKVVPWIDVYARATCQPREVVVPLSMELMGNVVKQLVPSCVTVQRCGGCCPDDGLECVPTGQHQVRMQILMIQYPSSQLGEMSLEEHSQCECRPKKKESAVKPDSPRILCPPCTQRRQRPDPRTCRCRCRRRRFLHCQGRGLELNPDTCRCRKPRK

SEQ ID NO: 8 - protein, 207 aa, Mus musculus; identical to SEQ ID NO: 6

SEQ ID NO: 9 - protein, 13 aa, Artificial Sequence
  Description of Artificial Sequence Synthetic peptide
PXCVXXXRCXGCC

SEQ ID NO: 10 - DNA, 20 bp, Artificial Sequence
  Description of Artificial Sequence Synthetic primer
tctgagcatggaactcatgg

SEQ ID NO: 11 - DNA, 20 bp, Artificial Sequence
  Description of Artificial Sequence Synthetic primer
atcaccttcgagaagctgga

SEQ ID NO: 12 - DNA, 20 bp, Artificial Sequence
  Description of Artificial Sequence Synthetic primer
ccaaatctccacggtctgtt

SEQ ID NO: 13 - DNA, 23 bp, Artificial Sequence
  Description of Artificial Sequence Synthetic primer
cccacacccagctcacgtattta

SEQ ID NO: 14 - DNA, 20 bp, Artificial Sequence
  Description of Artificial Sequence Synthetic primer
caggctgctgtaacgatgaa

SEQ ID NO: 15 - DNA, 20 bp, Artificial Sequence
  Description of Artificial Sequence Synthetic primer
ggtgacctggatgagaagga

SEQ ID NO: 16 - DNA, 19 bp, Artificial Sequence
  Description of Artificial Sequence Synthetic primer
actcttccagccttccttc

SEQ ID NO: 17 - DNA, 20 bp, Artificial Sequence
  Description of Artificial Sequence Synthetic primer
ctgaccggcctgtatgctat

SEQ ID NO: 18 - DNA, 20 bp, Artificial Sequence
  Description of Artificial Sequence Synthetic primer
catgtcaagccagaggaaga

SEQ ID NO: 19 - DNA, 20 bp, Artificial Sequence
  Description of Artificial Sequence Synthetic primer
cccatgtgctcctaccagat

SEQ ID NO: 20 - DNA, 20 bp, Artificial Sequence
  Description of Artificial Sequence Synthetic primer
cagcatatgatggctgagtg

SEQ ID NO: 21 - DNA, 20 bp, Artificial Sequence
  Description of Artificial Sequence Synthetic primer
tttgcagggatcttcaactg

SEQ ID NO: 22 - DNA, 19 bp, Artificial Sequence
  Description of Artificial Sequence Synthetic primer
ggagccgtgaccactgaca

SEQ ID NO: 23 - DNA, 20 bp, Artificial Sequence
  Description of Artificial Sequence Synthetic primer
gacaaggcctcagggtacca

SEQ ID NO: 24 - DNA, 18 bp, Artificial Sequence
  Description of Artificial Sequence Synthetic primer
ccattctggcccaccaac

SEQ ID NO: 25 - DNA, 21 bp, Artificial Sequence
  Description of Artificial Sequence Synthetic primer
atatctacctgcggagtgaag

SEQ ID NO: 26 - DNA, 20 bp, Artificial Sequence
  Description of Artificial Sequence Synthetic primer
tcggaggctatggattcaac

SEQ ID NO: 27 - DNA, 20 bp, Artificial Sequence
  Description of Artificial Sequence Synthetic primer
acttgtttgagtgggccaag

SEQ ID NO: 28 - DNA, 21 bp, Artificial Sequence
  Description of Artificial Sequence Synthetic primer
cggttttgagaagcccatatt

SEQ ID NO: 29 - DNA, 21 bp, Artificial Sequence
  Description of Artificial Sequence Synthetic primer
cagtactcacctcagcagttg

SEQ ID NO: 30 - DNA, 20 bp, Artificial Sequence
  Description of Artificial Sequence Synthetic primer
ctgccaggacagtacccaag

SEQ ID NO: 31 - DNA, 20 bp, Artificial Sequence
  Description of Artificial Sequence Synthetic primer
gccacaaaccctttgaaaaa

SEQ ID NO: 32 - DNA, 20 bp, Artificial Sequence
  Description of Artificial Sequence Synthetic primer
agccctctgcactgtatgct

SEQ ID NO: 33 - DNA, 21 bp, Artificial Sequence
  Description of Artificial Sequence Synthetic primer
ggcactaagtgccctcaacct

SEQ ID NO: 34 - DNA, 21 bp, Artificial Sequence
  Description of Artificial Sequence Synthetic primer
ggatcatccagcagcctttga

SEQ ID NO: 35 - DNA, 24 bp, Artificial Sequence
  Description of Artificial Sequence Synthetic primer
cccagcagaataaagctactttgg

SEQ ID NO: 36 - DNA, 20 bp, Artificial Sequence
  Description of Artificial Sequence Synthetic primer
actcttgccacacaggctct

SEQ ID NO: 37 - DNA, 25 bp, Artificial Sequence
  Description of Artificial Sequence Synthetic primer
tcaatgtaccaggaattacagaagg

SEQ ID NO: 38 - DNA, 20 bp, Artificial Sequence
  Description of Artificial Sequence Synthetic primer
catgagctaaaccaccaggg

SEQ ID NO: 39 - DNA, 18 bp, Artificial Sequence
  Description of Artificial Sequence Synthetic primer
cgcaggctctgaacctgg

SEQ ID NO: 40 - DNA, 20 bp, Artificial Sequence
  Description of Artificial Sequence Synthetic primer
gcaagtcccatcagcaactg

SEQ ID NO: 41 - DNA, 22 bp, Artificial Sequence
  Description of Artificial Sequence Synthetic primer
gtggtcagagattccaggttcc

SEQ ID NO: 42 - DNA, 24 bp, Artificial Sequence
  Description of Artificial Sequence Synthetic primer
tacaaccaagtggtgacatctctg

SEQ ID NO: 43 - DNA, 27 bp, Artificial Sequence
  Description of Artificial Sequence Synthetic primer
gatgagcataggacatacttagatgtg

SEQ ID NO: 44 - DNA, 20 bp, Artificial Sequence
  Description of Artificial Sequence Synthetic primer
ccagaaagggaaggacatca

SEQ ID NO: 45 - DNA, 21 bp, Artificial Sequence
  Description of Artificial Sequence Synthetic primer
ttcagctgggaatagagttcg

SEQ ID NO: 46 - DNA, 20 bp, Artificial Sequence
  Description of Artificial Sequence Synthetic primer
gatggtgacaagctggtggt

SEQ ID NO: 47 - DNA, 20 bp, Artificial Sequence
  Description of Artificial Sequence Synthetic primer
ggaagatggcgcctggtgga

SEQ ID NO: 48 - DNA, 17 bp, Artificial Sequence
  Description of Artificial Sequence Synthetic primer
cttcaccctgcccgagg

SEQ ID NO: 49 - DNA, 20 bp, Artificial Sequence
  Description of Artificial Sequence Synthetic primer
agagacggtcttgtcgcagt

SEQ ID NO: 50 - DNA, 20 bp, Artificial Sequence
  Description of Artificial Sequence Synthetic primer
ggaggagtacaacaccacgg

SEQ ID NO: 51 - DNA, 20 bp, Artificial Sequence
  Description of Artificial Sequence Synthetic primer
agctggagaatgagctggag

SEQ ID NO: 52 - DNA, 20 bp, Artificial Sequence
  Description of Artificial Sequence Synthetic primer
cgtgaggaggaggagaacag

SEQ ID NO: 53 - DNA, 21 bp, Artificial Sequence
  Description of Artificial Sequence Synthetic primer
agcacctctctcgtgatttcc

SEQ ID NO: 54 - DNA, 20 bp, Artificial Sequence
  Description of Artificial Sequence Synthetic primer
ggagctactgggctgtgaag

SEQ ID NO: 55 - DNA, 22 bp, Artificial Sequence
  Description of Artificial Sequence Synthetic primer
tctcccctctctacgcattcta

SEQ ID NO: 56 - DNA, 20 bp, Artificial Sequence
  Description of Artificial Sequence Synthetic primer
tgcgacttcaacagcaactc

SEQ ID NO: 57 - DNA, 20 bp, Artificial Sequence
  Description of Artificial Sequence Synthetic primer
tctgcattcacattggctgt

SEQ ID NO: 58 - DNA, 20 bp, Artificial Sequence
  Description of Artificial Sequence Synthetic primer
acttcaccaccctgaagcaa

SEQ ID NO: 59 - DNA, 20 bp, Artificial Sequence
  Description of Artificial Sequence Synthetic primer
ccaggtgatgcctttgttct

SEQ ID NO: 60 - DNA, 23 bp, Artificial Sequence
  Description of Artificial Sequence Synthetic primer
tcccctctacatgccttcaatgc

SEQ ID NO: 61 - DNA, 20 bp, Artificial Sequence
  Description of Artificial Sequence Synthetic primer
tatgtgctggctttggtgat

SEQ ID NO: 62 - DNA, 20 bp, Artificial Sequence
  Description of Artificial Sequence Synthetic primer
ttcagcttgtggatgtgctc

SEQ ID NO: 63 - DNA, 20 bp, Artificial Sequence
  Description of Artificial Sequence Synthetic primer
atctccttctgcatcctgtc

SEQ ID NO: 64 - DNA, 20 bp, Artificial Sequence
  Description of Artificial Sequence Synthetic primer
ccgttcttcagcatttggat

SEQ ID NO: 65 - DNA, 20 bp, Artificial Sequence
  Description of Artificial Sequence Synthetic primer
tggtaggagagcagcacctt

SEQ ID NO: 66 - DNA, 20 bp, Artificial Sequence
  Description of Artificial Sequence Synthetic primer
ccttgaagaagcgacctttg

SEQ ID NO: 67 - DNA, 20 bp, Artificial Sequence
  Description of Artificial Sequence Synthetic primer
gtggtttatccgctggtatg

SEQ ID NO: 68 - DNA, 20 bp, Artificial Sequence
  Description of Artificial Sequence Synthetic primer
ccctttgtacaaggaggtga

SEQ ID NO: 69 - DNA, 20 bp, Artificial Sequence
  Description of Artificial Sequence Synthetic primer
tggtttgctgcatggttctg

SEQ ID NO: 70 - DNA, 19 bp, Artificial Sequence
  Description of Artificial Sequence Synthetic primer
gccgaatagttcgccgaaa

SEQ ID NO: 71 - DNA, 21 bp, Artificial Sequence
  Description of Artificial Sequence Synthetic primer
aatgcgagtggtcttccatca

SEQ ID NO: 72 - DNA, 22 bp, Artificial Sequence
  Description of Artificial Sequence Synthetic primer
ccttcccaagtttcaacaagtc

SEQ ID NO: 73 - DNA, 20 bp, Artificial Sequence
  Description of Artificial Sequence Synthetic primer
cagcctctgaatttgtgcag

SEQ ID NO: 74 - DNA, 20 bp, Artificial Sequence
  Description of Artificial Sequence Synthetic primer
agagattcggcctctctgtg

SEQ ID NO: 75 - DNA, 22 bp, Artificial Sequence
  Description of Artificial Sequence Synthetic primer
attctgtttccttccttccaca

SEQ ID NO: 76 - DNA, 21 bp, Artificial Sequence
  Description of Artificial Sequence Synthetic primer
cacacaagttctcgagtcaag

SEQ ID NO: 77 - DNA, 20 bp, Artificial Sequence
  Description of Artificial Sequence Synthetic primer
gccacaaaccctttgaaaaa

SEQ ID NO: 78 - DNA, 20 bp, Artificial Sequence
  Description of Artificial Sequence Synthetic primer
tacaaggggttcatgccttc

SEQ ID NO: 79 - DNA, 20 bp, Artificial Sequence
  Description of Artificial Sequence Synthetic primer
cagaaaggagggcacaaatc

SEQ ID NO: 80 - DNA, 23 bp, Artificial Sequence
  Description of Artificial Sequence Synthetic primer
gccacatagatctctgccagtgt

SEQ ID NO: 81 - DNA, 18 bp, Artificial Sequence
  Description of Artificial Sequence Synthetic primer
accgggacctgcacctgt

SEQ ID NO: 82 - DNA, 23 bp, Artificial Sequence
  Description of Artificial Sequence Synthetic primer
tccttttgtgcaactaggaacgt

SEQ ID NO: 83 - DNA, 20 bp, Artificial Sequence
  Description of Artificial Sequence Synthetic primer
ccttgccctgtcaggtatgt

SEQ ID NO: 84 - DNA, 20 bp, Artificial Sequence
  Description of Artificial Sequence Synthetic primer
gagtgagaagtcgcctgcac

SEQ ID NO: 85 - DNA, 25 bp, Artificial Sequence
  Description of Artificial Sequence Synthetic primer
ttcctgaggatacaagataccattg

SEQ ID NO: 86 - DNA, 21 bp, Artificial Sequence
  Description of Artificial Sequence Synthetic primer
tcgaaggtctccagacaggag

SEQ ID NO: 87 - DNA, 20 bp, Artificial Sequence
  Description of Artificial Sequence Synthetic primer
gggggaaatcacagcttctc

SEQ ID NO: 88 - DNA, 21 bp, Artificial Sequence
  Description of Artificial Sequence Synthetic primer
gctataccagcatgtccgctc

SEQ ID NO: 89 - DNA, 21 bp, Artificial Sequence
  Description of Artificial Sequence Synthetic primer
aatctcttcggtcaatgggac

SEQ ID NO: 90 - DNA, 22 bp, Artificial Sequence
  Description of Artificial Sequence Synthetic primer
caccactccaatcccaagtaag

SEQ ID NO: 91 - DNA, 20 bp, Artificial Sequence
  Description of Artificial Sequence Synthetic primer
gtctccagttcgcactcctc

SEQ ID NO: 92 - DNA, 20 bp, Artificial Sequence
  Description of Artificial Sequence Synthetic primer
ctgcacatggatgagtttgc

SEQ ID NO: 93 - DNA, 20 bp, Artificial Sequence
  Description of Artificial Sequence Synthetic primer
aatttccatccaggcctctt

SEQ ID NO: 94 - DNA, 20 bp, Artificial Sequence
  Description of Artificial Sequence Synthetic primer
ccgagtacaggtgacattgt

SEQ ID NO: 95 - DNA, 21 bp, Artificial Sequence
  Description of Artificial Sequence Synthetic primer
aacactgctgagtcctttccc

SEQ ID NO: 96 - DNA, 20 bp, Artificial Sequence
  Description of Artificial Sequence Synthetic primer
tactgggcttcgagagcatt

SEQ ID NO: 97 - DNA, 20 bp, Artificial Sequence
  Description of Artificial Sequence Synthetic primer
ttgaggagctttcaccgaac

SEQ ID NO: 98 - DNA, 20 bp, Artificial Sequence
  Description of Artificial Sequence Synthetic primer
gcagccgcattaagttcttc

SEQ ID NO: 99 - DNA, 20 bp, Artificial Sequence
  Description of Artificial Sequence Synthetic primer
tcctctctgccaggatcttc

SEQ ID NO: 100 - DNA, 22 bp, Artificial Sequence
  Description of Artificial Sequence Synthetic primer
agtaaaagcagggagtctgtgg

SEQ ID NO: 101 - DNA, 20 bp, Artificial Sequence
  Description of Artificial Sequence Synthetic primer
cctcctgtgagctggaagtc

SEQ ID NO: 102 - DNA, 22 bp, Artificial Sequence
  Description of Artificial Sequence Synthetic primer
acggattggaagttctattggc

SEQ ID NO: 103 - DNA, 20 bp, Artificial Sequence
  Description of Artificial Sequence Synthetic primer
gcctctcttgctcagtgtcc

SEQ ID NO: 104 - DNA, 20 bp, Artificial Sequence
  Description of Artificial Sequence Synthetic primer
atcaccttcgagaagctgga

SEQ ID NO: 105 - DNA, 20 bp, Artificial Sequence
  Description of Artificial Sequence Synthetic primer
acttcaccaccctgaagcaa

SEQ ID NO: 106 - DNA, 20 bp, Artificial Sequence
  Description of Artificial Sequence Synthetic primer
ccaaatctccacggtctgtt

SEQ ID NO: 107 - DNA, 20 bp, Artificial Sequence
  Description of Artificial Sequence Synthetic primer
ccaggtgatgcctttgttct

SEQ ID NO: 108 - DNA, 20 bp, Artificial Sequence
  Description of Artificial Sequence Synthetic primer
tctgagcatggaactcatgg

SEQ ID NO: 109 - DNA, 20 bp, Artificial Sequence
  Description of Artificial Sequence Synthetic primer
tctgcattcacattggctgt

SEQ ID NO: 110 - DNA, 18 bp, Artificial Sequence
  Description of Artificial Sequence Synthetic primer
cgcaggctctgaacctgg

SEQ ID NO: 111 - DNA, 21 bp, Artificial Sequence
  Description of Artificial Sequence Synthetic primer
tcgaaggtctccagacaggag

SEQ ID NO: 112 - protein, 6 aa, Artificial Sequence
  Description of Artificial Sequence Synthetic 6xHis tag
HHHHHH

(19) United States
(12) Patent Application Publication
(10) Pub. No.: US 2011/0150900 A1
(43) Pub. Date: Jun. 23, 2011

(54) REGULATION OF FATTY ACID TRANSPORTERS

(75) Inventor: Ulf Eriksson, Stockholm (SE)

(73) Assignee: LUDWIG INSTITUTE FOR CANCER RESEARCH, NEW YORK, NY (US)

(21) Appl. No.: 12/937,164

(22) PCT Filed: Apr. 8, 2009
(86) PCT No.: PCT/US09/39885

§ 371 Date: Feb. 8, 2011

Related U.S. Application Data
(60) Provisional application No. 61/123,523, filed on Apr. 9, 2008.

(51) Int. Cl. A61K 39/395; A61K 31/713; A61P 3/00; G01N 33/53

(52) U.S. Cl. 424/158.1; 514/44 A; 435/7.21

(57) ABSTRACT

The present invention provides materials and methods for modulating FATP expression and/or activity in vivo. The materials and methods have numerous diagnostic, prophylactic, and therapeutic applications for various diseases and conditions that are influenced by FATPs, or characterized by excessive or inadequate FATP expression or activity.

### CROSS REFERENCE TO RELATED APPLICATION

This application claims the priority of U.S. Provisional Application Ser. No. 61/123,523 filed Apr. 9, 2008, the disclosure of which is incorporated herein by reference in its entirety.

### BACKGROUND OF THE INVENTION

Fatty acid transport proteins (FATP) are membrane-bound proteins that transport free fatty acids (FFA) across the plasma membrane, and are thus responsible for cellular uptake of FFA from the extracellular space. Transgenic overexpression of FATP1 in the heart leads to increased lipid uptake, lipotoxicity, and cardiac myopathy (Chiu et al. (2005) Circ. Res. 96:225-233). Chiu et al. illustrate the critical need for a controlled uptake of FFA to tissues. Accumulated FFA are not properly oxidized and can be toxic to cells.

Lipid accumulation in tissues, particularly of skeletal muscle, has also been suggested to induce insulin resistance syndrome also known as the diabetic metabolic syndrome. FATP4 has been shown by linkage analysis to be a candidate gene in the insulin resistance syndrome characterized by dyslipidemia, hypertension, and the procoagulant state (Gertow et al. (2004) Clin. Endocrinol. Metab. 89:392-399). Similarly, FATP1 has been linked with elevated post-prandial lipaemia and alterations in LDL particle size distribution (Gertow et al. (2003) Atherscler. 167:265-273). A review of fatty acid transport proteins and their role in insulin resistance can be found in Fisher et al. (2005) Curr. Opin. Lipidol. 16:173-178.

Aberrant lipid accumulation is closely related to deleterious lipotoxic effects, insulin resistance, perturbations of both lipid and carbohydrate metabolism and other features of the metabolic syndrome (Unger et al. (2004) Endocrinol. 144:5159-5164). Thus, a need exists for new materials and methods for modulating FATP expression and/or activity in vivo, for prophylaxis or therapy of various diseases and conditions that are influenced by FATPs.

The maintenance of normal physiological and metabolic activities in peripheral organs is dependent on efficient supply of nutrients and oxygen by the vasculature. The organs further need to adapt their metabolism and uptake of energy-rich substrates from plasma to changes in nutrient availability, hormonal milieu and physical activity.

The family of vascular endothelial VEGFs, comprising VEGF-A, PlGF and VEGF-B, are major regulators of blood vessel growth and development, and are involved in several other aspects of endothelial cell (EC) physiology. VEGF-A is required for vasculogenesis and angiogenesis (Carmeliet et al. (1996) Nat Med 7:575-583; Ferrara et al. (1996) Nature 380:439-442) and PlGF has a modifying role in angiogenesis.

VEGF-B is widely expressed, being most abundant in heart, skeletal muscle, brown adipose tissue (BAT) and brain (Olofsson et al. (1996) Proc Natl Acad Sci USA 93:2576-2581). It is expressed as two isoforms, VEGF-B₁₆₇ and VEGF-B₁₈₆, which differ in their biochemical properties due to alternative splicing of the C-terminal region (Olofsson et al. (1996) J Biol Chem 271:19310-19317). Both VEGF-B isoforms bind VEGF receptor 1 (VEGFR1) and neuropilin-1 (NRP-1), but not VEGFR2 or 3 (Makinen et al. (1999) J Biol Chem 274:21217-21222; Olofsson et al. (1998) Proc Natl Acad Sci USA 95:11709-11714). The ability of VEGF-B to stimulate angiogenesis is poor in most tissues except heart (Lahteenvuo et al. (2009) Circulation 119:845-856; Li et al. (2008) Arterioscler Thromb Vasc Biol 28:1614-1620), and VEGF-B, unlike VEGF-A, does not induce vascular permeability (Nagy et al. (2008) Angiogenesis 11:109-119). VEGF-B and PlGF share the same receptor, VEGFR1, for signal transduction. VEGF-B deficient (VEGF-B^{-/-}) mice are healthy and fertile, and only minor cardiac abnormalities have been observed (Aase et al. (2001) Circulation 104:358-364; Bellomo et al. (2000) Circ Res 86:E29-35). Recently, a transgenic mouse model overexpressing VEGF-B₁₆₇ specifically in the heart was reported to develop hypertrophy of cardiomyocytes, characterized by ceramide accumulation in the tissue, increased mitochondrial lysis, cardiomyopathy and eventually increased mortality (Karpanen et al. (2008) Circ Res 103:1018-1026).

In accordance with the present invention, a novel important link between endothelial function and peripheral organ metabolism has been discovered, whereby the metabolic needs of a tissue can regulate EC-mediated uptake of long chain fatty acids (LCFAs).

### SUMMARY OF THE INVENTION

The present invention provides materials and methods for modulating FATP expression and/or activity in vivo. The materials and methods have numerous diagnostic, prophylactic, and therapeutic applications for various diseases and conditions that are influenced by FATPs, or characterized by excessive or FATP expression or activity.

For example, in one embodiment, the invention provides a method of reducing lipid accumulation in any mammalian subject that has a disease or condition for which lipid reduction is a desirable therapeutic goal (as well as for subjects at risk for developing such disease or condition). In one variation, the method comprises administering to a mammalian subject in need of treatment to reduce lipid accumulation a composition that comprises a VEGF-B inhibitor, in an amount effective to reduce lipid accumulation in a tissue in the subject.

In these and other embodiments of the invention, the method optionally further includes one or more steps of monitoring one or more parameters in the subject, to confirm the safety and/or efficacy of the treatment and/or to optimize dosing. Thus, for example, in a method of the invention for reducing lipid accumulation, the method optionally further comprising monitoring lipid markers in a biological sample comprising a tissue or fluid from the subject. Exemplary tissues include biopsies of any organ (liver, kidney, spine, heart, vessel, intestine, etc) or skin or hair, for example. Exemplary fluids include blood, cerebrospinal fluid, semen, saliva, or other secretions or excretions.

Another aspect of the invention is directed to a method of stimulating glucose metabolism in a mammalian subject, comprising administering to a mammalian subject in need of treatment for a condition characterized by elevated blood glucose a composition that comprises a VEGF-B inhibitor, in an amount effective to increase glucose tolerance and/or reduce insulin resistance in the subject. Glucose metabolism generally may be tracked via glucose measurements in the blood or serum of the subject. In some variations, the method further includes monitoring glucose levels in the blood of the subject. The administration (dose, dosing frequency) is modulated to balance maximum therapeutic benefit with minimization of side-effects.

For methods described herein which involve a subject in need of treatment or prophylaxis for a particular condition, the method of the invention may include selecting a patient based on appropriate criteria (e.g., medical diagnosis based on accepted tests, family history, examination, and other criteria) for the prophylaxis or therapy. Thus, for example, the methods summarized above may further comprise selecting for treatment a subject with at least one disease or condition selected from the group consisting of: obesity, insulin resistance, diabetes, hepatic steatosis, cardiovascular disease and metabolic syndrome. In some preferred variations, the subject has type II diabetes.

Numerous exemplary VEGF-B inhibitors are described below in greater detail, for use in methods of the invention that require them. Exemplary VEGF-B inhibitors include, but are not limited to:

(a) antibodies that immunoreact (bind) with VEGF-B (also known as VEGF-B antibodies or anti-VEGF-B antibodies); such antibodies include antibodies that bind multiple isoforms of VEGF-B, as well as antibodies that show preferential binding or specificity for one isoform, such as a VEGF-B₁₆₇ antibody or a VEGF-B₁₈₆ antibody;

(b) a VEGFR-1 antibody (preferably one that immunoreacts with the extracellular domain of VEGFR-1);

(c) fragments of (a) or (b) that retain antigen binding activity;

(d) polypeptides that comprise an antigen binding domain of (a), (b) or (c) and that bind said antigen;

(e) antisense oligonucleotides that inhibit VEGF-B transcription or translation;

(e) aptamers that inhibit VEGF-B₁₆₇ and/or VEGF-B₁₈₆;

(f) short interfering RNA (siRNA, RNAi) that inhibits VEGF-B translation; and,

(g) small molecule inhibitors of VEGFR-1.

Antibodies and other binding agents (and antigen binding domains thereof) for use as described herein bind selectively to antigen (such as VEGF-B), which means they bind preferentially to antigen with a greater binding affinity than with which they bind other antigens. In some variations, the antibodies or other binding agents are antigen-specific binding agents that are capable of distinguishing antigen from other closely related members of the same family of proteins (e.g., other VEGF/PDGF family members). Typically, the antigen binding agents for use in practicing the invention (or fragments, variants, or derivatives thereof of) will bind with a greater affinity to human antigen (when selected for human treatment/medicaments) as compared to its binding affinity to antigen of other, e.g, non human, species. However, it is to be expected that antibodies that are specific for a human protein (e.g., VEGF-B or VEGFR-1) may also bind, to varying degrees, to species homologs of the same protein.

The term “antigen binding domain” or “antigen binding region” refers to that portion of the selective binding agent (such as an antibody molecule) which contains the specific binding agent amino acid residues that interact with an antigen and confer on the binding agent its specificity and affinity for the antigen. In an antibody, the antigen binding domain is commonly referred to as the “complementarity determining region”, or “CDR.”

As used herein, the term “VEGF-B” or “VEGF-B polypeptide” should be understood to mean a soluble form of VEGF-B that retains a characteristic VEGF-B biological activity, such as binding to VEGFR-1 expressed on the surface of cells. Another characteristic VEGF-B activity that is important to the invention is VEGF-B's effects on modulating FATP proteins, described herein in detail. The term VEGF-B polypeptide likewise refers to pro-forms of the polypeptide that may be cleaved in vivo into active forms; and polypeptides that are chemically modified, e.g., to improve serum half-life or stability. The term “VEGF-B polynucleotide” refers to polynucleotides that encode a VEGF-B polypeptide. Such polynucleotides include “naked DNA” constructs, and also various vectors that contain/include the encoding polynucleotide sequence. The polynucleotide preferably includes one or more suitable expression control sequences, such as promoters, to promote expression of the encoded polypeptide in cells of the mammalian subject to be treated.

In some variations, a ligand (e.g., polypeptides, antibodies, etc.) may be coated with beads and administered in vivo as described in Ueng et al. (2006) J. Orthopedic Research 22:592-599 and Kumar (2000) J. Pharm. Pharmaceut. Sci. 3:234-258.

For aspects of the invention that involve VEGF-B polypeptides or polynucleotides, human polypeptide sequences are preferred, yet analogs that retain VEGF-B biological activity also are contemplated. Thus, in some variations, the agent comprises, for example:

(a) a polypeptide that comprises an amino acid sequence that is at least 90%, or 95%, or 97.5%, or 99% identical to the amino acid sequence of amino acids 22 to 188 of SEQ ID NO: 3, which constitutes a human sequence for VEGF-B₁₆₇;

(b) a polypeptide that comprises an amino acid sequence that is at least 90%, or 95%, or 97.5%, or 99% identical to the amino acid sequence of amino acids 22 to 207 of SEQ ID NO: 4, which constitutes a human sequence for VEGF-B₁₈₆;

(c) a polynucleotide that comprises a nucleotide sequence that encodes (a) or (b).

Exemplary polynucleotides for practicing the invention include expression vectors, such as “gene therapy” vectors, that comprise the polynucleotide that encodes the desired polypeptide. Exemplary expression vectors include adenoviral vectors, adeno-associated viral vectors, and lentivirus vectors. Replication-deficient forms of the vectors are preferred.

Numerous aspects of the invention have been described in the context of methods of treatment. For each such method, a related aspect of the invention is use of the agent (described in the method) for the manufacture of a medicament for treatment or prophylaxis for the specified disease or conditions.

For example, the invention includes use of a VEGF-B inhibitor in the manufacture of a medicament to reduce lipid accumulation in a mammalian subject.

The invention also includes use of a VEGF-B inhibitor in the manufacture of a medicament to stimulate glucose metabolism in a mammalian subject.

In related aspects, the invention includes use of a VEGF-B inhibitor in the manufacture of a medicament for the treatment or prophylaxis of a disease or condition selected from the group consisting of: obesity, insulin resistance, diabetes, hepatic steatosis, metabolic syndrome, lipid accumulation in the kidney, lipid accumulation in the heart or skeletal muscle, and lipid accumulation in other organs.

For all therapeutic and prophylactic methods described herein, exemplary mammalian subjects include humans and animals of economic importance for farming, food, livestock, transportation, pets, zoos, pre-clinical medical work. Exemplary animals include dogs/canines, cats/felines, primates, pigs/porcines, cows/bovines, horses/equines, rats, mice, dromedaries, and others.

Preferred compositions for administration further include one or more additional components such as pharmaceutically acceptable diluents, adjuvants, carriers, preservatives, flavorings, or other convention additives described herein and/or known in the field.

Additionally, co-therapies are contemplated as part of the invention. Administration of multiple therapeutics may be simultaneous (in admixture or separate), sequential, or separated in time during a treatment period.

Many of the diseases or conditions described herein are chronic in nature and the administration is expected to be repeated over a period that may involve days, weeks, months, years, or even the entire duration of a subject's life.

In jurisdictions that forbid the patenting of methods that are practiced on the human body, the following restrictions are intended: (1) the selecting of a human subject shall be construed to be restricted to selecting based on testing of a biological sample that has previously been removed from a human body and/or based on information obtained from a medical history, patient interview, or other activity that is not practiced on the human body; and (2) the administering of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the selecting of subjects and the administering of compositions includes both methods practiced on the human body and also the foregoing activities.

Additional features and variations of the invention will be apparent to those skilled in the art from the entirety of this application, including the drawing and detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

In addition to the foregoing, the invention includes, as an additional aspect, all embodiments of the invention narrower in scope in any way than the variations specifically mentioned above. For example, although aspects of the invention may have been described by reference to a genus or a range of values for brevity, it should be understood that each member of the genus and each value or sub-range within the range is intended as an aspect of the invention. Likewise, various aspects and features of the invention can be combined, creating additional aspects which are intended to be within the scope of the invention.

### BRIEF DESCRIPTION OF THE DRAWINGS

FIG. 1A-F demonstrate reduced peripheral lipid uptake and storage in VEGF-B^{-/-} mice. FIG. 1A is a schematic illustration showing: I.) VEGF-B co-expression with mitochondrial proteins in order to coordinate lipid uptake and β-oxidation; II.) VEGF-B secretion by tissue cells, and signaling in a paracrine fashion to VEGFR1 and NRP-1 on ECs; III.) stimulation of ECs with VEGF-B leading to upregulation of endothelial FATP3 and FATP4; and (IV.) subsequent transport of LCFAs across the EC layer to the surrounding tissue. FIG. 1B shows the time-course of plasma radioactivity in wt and VEGF-B^{-/-} mice after oral gavage with ¹⁴C—OA (n=3). FIG. 1C shows plasma levels of TGs and NEFAs measured in fed or fasted wt and VEGF-B^{-/-} mice (n=10-15). FIG. 1D depicts the distribution of ¹⁴C—OA in organs from wt and VEGF-B^{-/-} mice 2 hrs and 24 hrs after oral gavage (n=3-5). Data is represented as mean+/−SEM. FIG. 1E shows ORO or HE staining of tissues from wt and VEGF-B^{-/-} mice. Scale bar, 50 μm (n=6-7). FIG. 1F shows mean uptake of [¹⁸F]FDG into wt and VEGF-B^{-/-} hearts. Infolded; representative PET image of hearts (arrowhead) after 60 min (n=3-4) *P<0.05, **P<0.01, ***P<0.001.

FIG. 2 depicts expression of the fatty acid handling proteins Fatp4 and Cd36 in the small intestine of wt and VEGF-B^{-/-} mice. 8-12 week old male wt and VEGF-B^{-/-} mice were sacrificed and the proximal half of the small intestine was dissected and rinsed with PBS. RNA was isolated using RNeasy kit (Qiagen) as described in Example 1. The expression of Fatp4 and Cd36 was analyzed by qPCR and the results presented as fold-change versus the level in wt mice, which was set to 1 unit (n=3-6 mice per genotype).

FIG. 3 shows Wt and VEGF-B^{-/-} mice plasma levels of glucose, insulin and glucagon, measured in fed and fasted mice. Glucose was measured from the tail vein of normally fed or over-night fasted age-matched 8-12 week old male mice using a Contour Blood Glucose Meter (Ascensia). For all the other analyses, blood was collected from hearts of Avertin-anesthetized mice using EDTA syringes, and centrifuged for 1 min at 16,000 g. Insulin and glucagon were measured using the Mercodia mouse insulin ELISA kit and the Multispecies Glucagon ELISA kit from Biovendor, respectively, according to the manufacturers' instructions (n=10-15 mice per genotype).

FIG. 4 shows accumulation of the ¹⁴C—OA tracer in different WAT depots. Relative radioactivity in retroperitoneal (rWAT), inguinal (iWAT) and subcutaneous WAT (scWAT) in VEGF-B^{-/-} mice as compared to controls 24 hrs after oral gavage, was performed as described in Example 1. (n=4 mice). *P<0.05, **P<0.01.

FIG. 5 depicts quantitation of ORO staining in heart and muscle, and of mean lipid vacuole size in iBAT from wt and VEGF-B^{-/-} animals, performed as described in Example 1. (n=4-7 mice per genotype). **P<0.01, ***P<0.001.

FIG. 6 shows mean radioactivity in dissected mouse hearts 60 min after [¹⁸F]FDG i.v. injection as percentage of injected [¹⁸F]FDG. dose (n=3-4 mice). *P<0.05.

FIGS. 7A-H show that VEGF-B^{-/-} mice have increased ketosis, accumulation of WAT and lower plasma leptin. FIG. 7A shows mean VO₂ consumption and VCO₂ release during 23 hrs for wt (left panel) and VEGF-B^{-/-} mice (right panel) as measured by indirect calorimetry. The mean RQ for three time periods (13.00-19.00; 19.00-07.00; 07.00-10.00) for each genotype is indicated in the graphs (n=6-7). FIG. 7B shows plasma levels of β-hydroxybutyrate measured in fed and fasted wt and VEGF-B^{-/-} mice (n=4-6). FIG. 7C is a graph of ex vivo β-oxidation in isolated organs from wt and VEGF-B^{-/-} mice. Dissected organs were weighed and incubated with ¹⁴C—OA, after which formed ¹⁴CO₂ was collected onto alkalinized membranes and analyzed by liquid scintillation (n=5). FIG. 7D shows coronal MRI images of wt and VEGF-B^{-/-} mice. The white signal represents adipose tissue. Quantification of total body fat from the images is shown to the right (n=6-7). FIG. 7E shows glycerol release measured ex vivo from eWAT that was isolated and incubated in DMEM without serum for 12 hrs (n=4-5). FIG. 7F shows HE staining of eWAT and ORO staining of liver from wt and VEGF-B^{-/-} mice. Scale bar, 50 μm (n=3-7). FIG. 7G depicts plasma levels of leptin measured in fed wt and VEGF-B^{-/-} mice (n=7-9). FIG. 7H shows mRNA levels of genes in lipid metabolism in eWAT and liver from VEGF-B^{-/-} and wt mice, determined by qPCR, and normalized by the mean value of wt mice set to 1 unit. (n=3) *P<0.05, **P<0.01, ***P<0.001.

FIGS. 8A-B show that VEGF-B^{-/-} animals have lower metabolic rate and voluntary activity. FIG. 8A shows mean VO₂ consumption and CO₂ output for wt and VEGF-B^{-/-} mice during the indicated time periods measured using indirect calorimetry as described in Example 1. (n=6-7). FIG. 8B shows mean voluntary activity for wt and VEGF-B^{-/-} mice during 4 days and nights measured as described by Aase et al. (2001) Circulation 104:358-364. and Johansson et al. (1997) Acta Physiol Scand 160:133-138. (n=4). *P<0.05, **P<0.01, ***P<0.001.

FIGS. 9A-C show that VEGF-B^{-/-} animals do not readily switch metabolism during 23 hrs. FIG. 9A shows mean RQ for wt and VEGF-B^{-/-} mice during the indicated times. The scale starts from 0.6 in order to better illustrate the differences between genotypes (n=6-7). FIGS. 9B-C are graphs of RQ (B) and measured VO₂ and CO₂ (C) for one representative individual mouse of each genotype during 23 hrs. **P<0.01, ***P<0.001.

FIG. 10 is a graph showing that VEGF-B^{-/-} mice have larger fat pads. Weight of epididymal (eWAT), retroperitoneal (rWAT), inguinal (iWAT) and subcutaneous WAT (scWAT) fat pads from 16-18 week old male wt and VEGF-B^{-/-} mice are normalized to body weight (n=5-7). *P<0.05, **P<0.01.

FIGS. 11A-C show size distribution and mean cell size of adipocytes in eWAT, and quantitation of ORO staining in liver from wt or VEGF-B^{-/-} animals. For FIG. 11A, adipose tissue from 12 week old male mice was prepared and stained by HE as described in Example 1. (n=3 animals per genotype). From 10 photographed frames per adipose tissue sample, 20 adipocytes/frame were chosen in blind for measurement of their diameters, giving a total of 200 measured adipocytes per mouse, or 600 measured adipocytes per genotype. Cells were measured in 5 mm bundles by hand on printed frames. Data show percentage of cells of a certain size of the total number of measured cells per genotype. FIG. 11B shows mean adipoyte cell diameter calculated from the same measurements as in A. FIG. 11C shows quantification of liver ORO staining performed as described in Example 1. (n=4-7 mice per genotype). **P<0.01.

FIGS. 12A-I show that expression of endothelial Fatp3 and Fatp4 is significantly reduced in VEGF-B^{-/-} organs. FIGS. 12A-C show mRNA levels of FATPs in wt and VEGF-B^{-/-} heart, oxidative skeletal muscle and liver. (n=3). FIGS. 12D-E show immunoblotting of the vascular FATPs in heart (D) and liver (E). (n=3). FIG. 12F shows expression of FATPs in isolated cell fractions from wt hearts (n=4). FIG. 12G shows mRNA levels of FATPs in isolated EC and non-EC fractions from wt and VEGF-B^{-/-} hearts. (n=3-4). FIGS. 12H-I show immunohistochemical localization of FATP3, FATP4 and type I myosin in hearts (H) and muscles (I) from wt and VEGF-B^{-/-} mice. Asterisks indicate type I fibres. Scale bars, 50 μm (n=2-3) *P<0.05, **P<0.01, ***P<0.001.

FIG. 13 shows mRNA expression of FATPs in wt and VEGF-B^{-/-} tissues. The mRNA levels of FATPs were determined by qPCR in iBAT (upper panel), and eWAT (lower panel) from wt (black bars) and VEGF-B−/− mice (white bars) as described in Example 1. (n=3). *P<0.05, **P<0.01.

FIG. 14 shows mRNA expression of Lpl in wt and VEGF-B^{-/-} tissues. The mRNA level of Lpl was determined by qPCR in heart, muscle, iBAT and liver from wt (black bars) and VEGF-B^{-/-} mice (white bars) as described in Example 1. (n=3). *P<0.05

FIG. 15 is an immunoblot of CD36 in heart lysates from wt and VEGF-B−/− mice. Calnexin was used as loading control. (n=3)

FIGS. 16A-B show mRNA expression of FATPs in wt mouse tissues (FIG. 16A), and cultured mouse endothelial cell lines (FIG. 16B). mRNA was extracted from tissues and cultured cells (brain-capillary derived bEnd3 ECs and mouse pancreatic MS-1 ECs) as described in Example 1, and analyzed by conventional RT-PCR using specific primers listed in Table 3.

FIG. 17 shows enrichment of ECs in isolated mouse heart EC and non-EC fractions, analyzed by conventional RT-PCR. Pecam1 and Vegfr1 were used as marker genes for ECs, and Myh6, Tnnt2 and Vegfb for cardiomyocytes. Total cDNA from heart was used as positive controls (+Ctl); negative controls (−Ctl) lack template. Myh6, cardiac myosin heavy polypeptide 6; Tnnt2, cardiac troponin T2.

FIG. 18 shows negative controls for immunohistochemistry. Staining of tissue sections with specific antibodies was performed as described in Example 1. For the negative controls the primary antibody was omitted.

FIGS. 19A-F show that VEGF-B induces FATP-expression in vivo and in vitro

FIG. 19A shows mRNA expression of Fatp3, Fatp4 and Pecam in mouse hearts transduced with adenoviruses encoding LacZ or different VEGFs. (n=3). FIG. 19B shows mRNA levels of FATPs in cultured bEnd3 ECs treated with different VEGFs. Data are represented as mean+/−SEM. FIG. 19C shows immunoblots of FATP4 in bEnd3 and MS-1 ECs after VEGF-B₁₈₆ treatment. FIG. 19D shows BODIPY-FA uptake into bEnd3 cells after transfection with the indicated plasmids±a 10× molar excess of OA (lower panel). Quantitation of the uptake is shown to the right. Data are represented as mean+/−SEM. FIG. 19E shows Fatp3 mRNA levels in bEnd3 cells treated with VEGF-B₁₈₆ and receptor neutralizing antibodies. Data are represented as mean+/−SEM. FIG. 19F shows Fatp3 mRNA levels in bEnd3 cells treated with VEGF-B₁₈₆ only, or together with 10× PlGF2. Data are represented as mean+/−SEM. Data in FIG. 19A are presented as fold-change versus AdLacZ transduced hearts set to 1 unit. Data in FIGS. 19B, C, E and F are presented as fold change versus sVEGFR1 treated cells. *P<0.05, **P<0.01, ***P<0.001.

FIGS. 20A-B show mRNA expression levels of Vegfb, Vegfa and Plgf after adenoviral transduction. In FIG. 20A, mouse and human Vegfb and Vegfa mRNA expression levels were analyzed by qPCR using previously published species-specific primers (Thijssen et al., (2004) Exp Cell Res 299: 286-293). Data are presented as endogenous levels of mVegfb and mVegfa in AdhVEGF-B and AdhVEGF-A transduced mouse hearts (dark grey bars), and as total Vegfb and Vegfa mRNA levels by combining the results obtained with specific mouse and human primers (light grey bars). In FIG. 20B, mouse Plgf mRNA expression was determined in AdLacZ and AdmPlGF transduced mouse hearts using primers specific for mPlgf transcripts. Primer sequences are found in Table 3. L19 was used as normalization gene, and data are shown as fold-change versus endogenous or AdLacZ levels set to 1 unit±SEM.

FIG. 21 shows mRNA levels of FATPs in cultured MS-1 ECs after treatment with different VEGFs. Cells were cultured, treated and results were analyzed as described in Example 1. Data are presented as fold-change versus sVEGFR1 treated cells set to 1 unit. *P<0.05, **P<0.01.

FIG. 22 shows mRNA expression of VEGFs and VEGF-receptors in cultured mouse endothelial cell lines. mRNA was extracted from cultured cells as described in Example 1, and analyzed by conventional RT-PCR using specific primers listed in Table 3.

FIGS. 23A-C shows that VEGF-B induced trans-endothelial lipid transport. FIG. 23A shows BODIPY-FA uptake into bEnd3 cells transfected with Fatp3 and/or Fatp4 targeting siRNA, and further treated with VEGF-B₁₈₆ (1^{st} row) or anti-VEGF-B mAb (2^{nd} row). Quantitation is shown to the right. Data are represented as mean+/−SEM. FIG. 23B shows kinetics of VEGF-B induced specific trans-endothelial ¹⁴C—OA transport. Data are represented as mean+/−SEM. FIG. 23C shows trans-endothelial transport of ¹⁴C—OA after treatment with the indicated VEGFs. Data are represented as mean+/−SEM. *P<0.05, **P<0.01, ***P<0.001

FIGS. 24A-C show efficiency of FATP knock-down by siRNA in bEnd3 cells. mRNA expression levels of Gapdh (FIG. 24A), Fatp3 (FIG. 24B) and Fatp4 (FIG. 24C) in bEnd3 cells transfected with either control siRNA or specific targeting siRNA, and further treated with VEGF-B186 or anti-VEGF-B mAb as described in Example 1 are shown. Results are presented as percentage of expression relative to the normalization gene L19. Bars represent mean±SEM of 2-4 wells per treatment. **P<0.01, ***P<0.001.

FIGS. 25A-C show that VEGF-B treatment does not increase endothelial permeability. FIG. 25A shows measurements of trans-endothelial resistance (TER) over membranes that were unseeded (No cells) or seeded with bEnd3 cells (Cells). Cells were subsequently treated with VEGFs or mock treated, and TER was measured before treatment, as well as after 5 hrs and 30 hrs of treatment, respectively. Data are shown as mean of measured TER±SEM of four wells per treatment. FIG. 25B shows trans-endothelial leakage assessed by adding ¹⁴C-labeled inulin to bEnd3 monolayers after treatment with VEGFs for 30 hrs. Data show mean±SEM of 4 independent experiments. FIG. 25C shows trans-endothelial transport of LCFAs across bEnd3 cell layers. ¹⁴C-labeled OA was added to cell culture inserts unseeded or seeded with bEnd3 cells. Bars represent mean±SEM of 3 independent experiments. Transport was determined by measuring the radioactivity in the lower compartments. *P<0.05, **P<0.01, ***P<0.001.

FIG. 26 shows expression of mitochondrial genes and Vegfb in hearts from control and db/db mice.

FIG. 27 shows starved blood glucose in db/db mice after six weeks of treatment with anti-VEGF-B antibody.

FIG. 28 shows fasting blood glucose in individual db/db mice after six weeks of treatment with anti-VEGF-B antibody.

FIG. 29 shows body weight curves for db/db mice treated with 2H10 or control C44 antibody.

FIG. 30 shows non-esterified fatty acids in plasma in db/db mice treated with 2H10 or control C44 antibody.

FIG. 31 shows glucose levels for individual mice treated with 2H10 or control C44 antibody.

FIG. 32 shows blood glucose levels in VEGF-B^{-/-} mice on a db/db background and littermate controls (2 mice and 3 littermate controls).

### DETAILED DESCRIPTION OF THE INVENTION

The FATP family of proteins comprises six members (FATP1-6) with distinct expression patterns. Two of the most relevant tissues to consider in terms of fatty acid metabolism and insulin resistance are adipose tissue and skeletal muscle. FATP1 expression is high in both tissues, whereas FATP4 is much lower (its principal site of expression being the small intestine), but both have been linked to insulin resistance and associated parameters. FATP1 has been linked to insulin resistance and is expressed in adipose tissue and skeletal muscle (Kim et al, J. Clin. Invest., 113:756-763, 2004). FATP2 is located in peroxisomes of the liver and kidney and is implicated in the activation of both bile acids and fatty acids (Mihalik et al., J. Biol. Chem., 277:24771-24779, 2002). FATP4 has also been linked to insulin-resistance related parameters and is expressed in the intestines, liver and adipose tissue. FATPs 2, 3 and 5 are expressed in the liver and kidney. FATP6 is expressed almost exclusively in the heart (Gimeno et al., J. Biol. Chem., 278:16039-16044, 2003).

The present invention demonstrates that VEGF-B has an unexpected role in lipid homeostasis. VEGF-B has an important role in controlling endothelial LCFA transport. As a consequence, VEGF-B^{-/-} animals have a defect in fatty acid uptake to peripheral tissues and reduced accumulation of intra-myocellular lipids. Bioinformatic analyses showed that Vegfb expression is tightly co-regulated with genes encoding proteins in the respiratory chain of mitochondria. In metabolically highly active tissues like heart and skeletal muscle, VEGF-B specifically controls accumulation of dietary fatty acids via regulation of two fatty acid transporters (FATP3 and FATP4) in endothelial cells of blood vessels. The co-expression of VEGF-B and components of mitochondria for energy production via β-oxidation of fatty acids defines a novel regulatory mechanism whereby lipid uptake and utilization are tightly coordinated. VEGF-B has a critical role in coordinating EC-mediated LCFA-uptake to the oxidative capacity of the mitochondria in the tissue. Altered lipid metabolism and fatty acid uptake underlie several human disorders including obesity, diabetes, and cardiovascular diseases and thus modulation of VEGF-B mediated signaling is useful for the treatment of such disorders.

Dietary LCFAs are transported in plasma as NEFAs bound to serum albumin, or as TGs packed into lipoproteins. Upon dissociation from albumin, or hydrolysis of the lipoprotein-bound TGs by LPL, free LCFAs have to transverse the endothelial cells of the blood vessels in order to be accumulated and metabolized by most tissues. Recently, PGC1α, a major regulator of metabolism, was shown to control the supply of angiogenic blood vessels to nutrient-deprived tissues through its regulation of VEGF-A, creating a link between vascular growth factors, nutrient delivery and oxidative metabolism (Arany et al. (2008) Nature 451:1008-1012). In accordance with the present invention it has been discovered that another vascular growth factor, VEGF-B, has a major role in peripheral fatty acid uptake and homeostasis, and that it acts via regulating transcytosis of LCFAs across the endothelium of tissues with high lipid turnover.

As disclosed herein, bioinformatic data shows that Vegfb is co-expressed with genes encoding mitochondrial proteins. This co-expression was previously indicated in a larger study characterizing mitochondrial proteins (Mootha et al. (2003) Cell 115:629-640). By co-expressing Vegfb and mitochondrial genes, it is proposed that parenchymal cells will ensure that the capacity for β-oxidation is matched with the uptake of LCFAs from the circulation, thereby avoiding intracellular accumulation of excess lipids and subsequent lipotoxicity (Muoio et al. (2007) Novartis Found Symp 286:24-38). This metabolic coupling integrates blood vessel function with the metabolic demands of the surrounding tissues.

As demonstrated herein, different FATPs are expressed in different cell types within an organ, and the endothelial FATPs have a crucial role for LCFA-uptake to heart and oxidative muscle. It is shown herein that FATP3 can induce cellular uptake of LCFAs. Studies using neutralizing antibodies showed that both VEGFR1 and NRP-1 are involved in VEGF-B mediated regulation of the endothelial FATPs.

As found in accordance with the present invention, VEGF-B^{-/-} mice have a complex metabolic phenotype due to several secondary adaptive changes in both liver and WAT, two organs that normally do not express high levels of VEGF-B. To summarize the VEGF-B^{-/-} phenotype, deficiency of VEGF-B primarily leads to lower LCFA-uptake and lipid accumulation in heart, muscle and BAT, as qPCR analysis showed very few other expressional changes in these organs. The lipids that are not taken up by muscles or heart are shunted to WAT, and VEGF-B deficient mice gain more weight than controls due to larger fat mass. The small hyperplastic adipocytes of the VEGF-B^{-/-} mice secrete less leptin, and lower leptin levels decrease lipolysis, and modulate PPARα activity and β-oxidation WAT and liver. The VEGF-B^{-/-} mice have a lower metabolic rate and lower activity during the night, which previously also has been correlated to low leptin levels (Friedman et al (1998) Nature 395:763-770).

Pathological accumulation of lipids is involved in inducing insulin resistance in peripheral tissues, and is a hallmark of type 2 diabetes (Savage et al. (2007) Physiol Rev 87:507-520). Cell fractionation studies of heart showed that Fatp1 was predominantly expressed in the myocyte fraction, thus acting downstream of the VEGF-B-regulated vascular FATPs. VEGF-B^{-/-} hearts also have an increased capacity for glucose uptake, as measured by PET, and the VEGF-B^{-/-} animals more efficiently shunt lipids to WAT.

In diabetes, VEGF-B may modulate the uptake of blood lipids to the endothelial cells, and aberrant expression (especially over-expression) may result in dyslipidemia, a condition found in many diabetic patients as part of the metabolic syndrome. In insulin-resistant states such as obesity, fatty acid metabolism and homeostasis are clearly disturbed. Fatty acid clearance and storage capacity in adipose tissue are impaired, and fatty acids may accumulate in the circulation and in nonadipose tissues such as liver skeletal muscle in the form of triacylglycerol, diacylglycerol, ceramides and fatty acyl-CoAs. Such ectopic lipid accumulation is closely related to deleterious lipotoxic effects, insulin resistance, perturbations of both lipid and carbohydrate metabolism and other features of the metabolic syndrome (Unger et al., Endocrinol., 144:5159-5164, 2004).

In cardiovascular disease, VEGF-B is known to stimulate revascularization following a heart infarction (see U.S. Patent Application Publication No.: 2003-0008824), and VEGF-B may have this effect by stimulating the uptake of FFA to the myocardium and indirectly stimulate revascularization by providing a better energy state in the myocardium.

Vascular endothelial growth factor B (VEGF-B) is abundantly expressed in several organs with a high metabolic turnover, like heart, skeletal muscle, brown fat, cerebral cortex and in gastric secreting parietal cells. It is well documented that all of these organs and cells use free fatty acids (FFA) as their main source for energy production under normal physiological conditions. The ultimate source of FFA for most cells is the plasma where serum albumin is the main transport protein. Plasma-derived FFA has to transverse the blood vessel wall to be used by the energy-requiring parenchymal cells of the heart, muscles, brown fat, brain, etc. (Van der Vusse et al., Adv. Exp. Med. Biol., 441:181-191, 1998).

VEGF-B binds to VEGF receptor 1 (VEGFR-1) which is mainly expressed by endothelial cells of the blood vessel wall. VEGF-B deficient animals are largely normal, suggesting that VEGF-B has no major role in the development of the vascular system during embryogenesis, nor has it an essential role in maintenance of the vascular system in adult animals. In certain stress situations (e.g., following an experimentally induced heart infarction), however, VEGF-B deficient animals do not recover and revascularize the infarcted zone of the heart as well as normal mice (U.S. Patent Application Publication No. 2005/0214280).

The term “VEGF-B” as used in the present invention encompasses those polypeptides identified as VEGF-B in U.S. Pat. No. 6,331,301, which is incorporated herein in its entirety, as well as published U.S. Application No. 2003/0008824. A human VEGF-B cDNA and deduced amino acid sequence are set forth in SEQ ID NOs: 1 and 2, respectively. A mouse VEGF-B cDNA and deduced amino acid sequence are set forth in SEQ ID NOs: 5 and 6, respectively.

VEGF-B comprises, but is not limited to, both the human VEGF-B₁₆₇ (SEQ ID NO: 3) and mouse VEGF-B₁₆₇ (SEQ ID NO: 7) and/or human VEGF-B₁₈₆ (SEQ ID NO: 4) and mouse VEGF-B₁₈₆ (SEQ ID NO: 8) isoforms or a fragment or analog thereof having the ability to bind VEGFR-1. Preferred active analogs exhibit at least 85% sequence identity, preferably at least 90% sequence identity, particularly preferably at least 95% sequence identity, and especially preferably at least 98% sequence identity to the natural VEGF-B polypeptides, as determined by BLAST analysis. The active substance typically will include the amino acid sequence Pro-Xaa-Cys-Val-Xaa-Xaa-Xaa-Arg-Cys-Xaa-Gly-Cys-Cys (where Xaa may be any amino acid) (SEQ ID NO: 9) that is characteristic of VEGF-B. The examples describe in vitro and in vivo assays for confirming that any selected VEGF-B analog has FATP modulating activity. Neuropilin-1 is also contemplated as having FATP modulating activity as it is a co-receptor for VEGF-B binding to VEGFR-1.

The sequences of SEQ ID NOs:1-8 are set forth in the Table below.

| Sequence | | |
|---|---|---|
| Identifier | Description | Sequence |
| 1. | VEGF-B | gcgatgcgggcgcccccggcgggcggccccggcgggcacca |
| | (human) DNA | tgagccctctgctccgccgcctgctgctcgccgcactcctg |
| | Genbank | cagctggcccccgcccaggcccctgtctcccagcctgatgc |
| | Acc. No.: | ccctggccaccagaggaaagtggtgtcatggatagatgtgt |
| | NM_003377 | atactcgcgctacctgccagccccgggaggtggtggtgccc |
| | | ttgactgtggagctcatgggcaccgtggccaaacagctggt |
| | | gcccagctgcgtgactgtgcagcgctgtggtggctgctgcc |
| | | ctgacgatggcctggagtgtgtgcccactgggcagcaccaa |
| | | gtccggatgcagatcctcatgatccggtacccgagcagtca |
| | | gctgggggagatgtccctggaagaacacagccagtgtgaat |
| | | gcagacctaaaaaaaaggacagtgctgtgaagccagacagg |
| | | gctgccactccccaccaccgtccccagccccgttctgttcc |
| | | gggctgggactctgcccccggagcaccctccccagctgaca |
| | | tcacccatcccactccagccccaggcccctctgcccacgct |
| | | gcacccagcaccaccagcgccctgacccccggacctgccgc |
| | | tgccgctgccgacgccgcagcttcctccgttgccaagggcg |
| | | gggcttagagctcaacccagacacctgcaggtgccggaagc |
| | | tgcgaaggtgacacatggcttttcagactcagcagggtgac |
| | | ttgcctcagaggctatatcccagtgggggaacaaagaggag |
| | | cctggtaaaaaacagccaagcccccaagacctcagcccagg |
| | | cagaagctgctctaggacctgggcctctcagagggctcttc |
| | | tgccatcccttgtctccctgaggccatcatcaaacaggaca |
| | | gagttggaagaggagactgggaggcagcaagaggggtcaca |
| | | taccagctcaggggagaatggagtactgtctcagtttctaa |
| | | ccactctgtgcaagtaagcatcttacaactggctcttcctc |
| | | ccctcactaagaagacccaaacctctgcataatgggatttg |
| | | ggctttggtacaagaactgtgacccccaaccctgataaaag |
| | | agatggaaggaaaaaaaaaaaaaaaaaaaaaaaaaaaaaaa |
| | | aaaaaaaaaaaaaaaaaaaaaaaa |
| 2. | VEGF-B | MSPLLRRLLLAALLQLAPAQAPVSQPDAPGHQRKVVSWIDV |
| | (human) AA | YTRATCQPREVVVPLTVELMGTVAKQLVPSCVTVQRCGGCC |
| | Genbank | PDDGLECVPTGQHQVRMQILMIRYPSSQLGEMSLEEHSQCE |
| | Acc. No.: | CRPKKKDSAVKPDRAATPHHRPQPRSVPGWDSAPGAPSPAD |
| | NM_003377 | ITHPTPAPGPSAHAAPSTTSALTPGPAAAAADAAASSVAKG |
| | | GA |
| 3. | VEGF-B₁₆₇ | MSPLLRRLLLAALLQLAPAQAPVSQPDAPGHQRKVVSWIDV |
| | (human) AA | YTRATCQPREVVVPLTVELMGTVAKQLVPSCVTVQRCGGCC |
| | Genbank | PDDGLECVPTGQHQVRMQILMIRYPSSQLGEMSLEEHSQCE |
| | Acc. No.: | CRPKKKDSAVKPDSPRPLCPRCTQHHQRPDPRTCRRRCRRR |
| | AAL79000 | SFLRCQGRGLELNPDTCRCRKLRR |
| 4. | VEGF-B₁₈₆ | MSPLLRRLLLAALLQLAPAQAPVSQPDAPGHQRKVVSWIDV |
| | (human) AA | VELMGTVAKQLVPSCVTVQRCGGCCPDDYTRATCQPREVVV |
| | Genbank | PLTGLECVPTGQHQVRMQILMIRYPSSQLGEMSLEEHSQCE |
| | Acc. No.: | CRPKKKDSAVKPDRAATPHHRPQPRSVPGWDSAPGAPSPAD |
| | AAL79001 | ITHPTPAPGPSAHAAPSTTSALTPGPAAAAADAAASSVAKG |
| | | GA |
| 5. | VEGF-B | ctcaggccgtcgctgcggcgctgcgttgcgctgcctgcgcc |
| | (mouse) DNA | cagggctcgggagggggccgcggaggagccgccccctgcgc |
| | Genbank | cccgccccgggtccccgggcccgcgccatggggctctggct |
| | Acc No.: | gccgccgccccccacgccgccgggctagggccatgcgggcg |
| | NM_011697 | ctcccggcgctcgccccccgcgggcaccatgagccccctgc |
| | | tccgtcgcctgctgcttgttgcactgctgcagctggctcgc |
| | | acccaggcccctgtgtcccagtttgatggccccagccacca |
| | | gaagaaagtggtgccatggatagacgtttatgcacgtgcca |
| | | catgccagcccagggaggtggtggtgcctctgagcatggaa |
| | | ctcatgggcaatgtggtcaaacaactagtgcccagctgtgt |
| | | gactgtgcagcgctgtggtggctgctgccctgacgatggcc |
| | | tggaatgtgtgcccactgggcaacaccaagtccgaatgcag |
| | | atcctcatgatccagtacccgagcagtcagctgggggagat |
| | | gtccctggaagaacacagccaatgtgaatgcagaccaaaaa |
| | | aaaaggagagtgctgtgaagccagacagggttgccataccc |
| | | caccaccgtccccagccccgctctgttccgggctgggactc |
| | | taccccgggagcatcctccccagctgacatcatccatccca |
| | | ctccagccccaggatcctctgcccgccttgcacccagcgcc |
| | | gtcaacgccctgacccccggacctgccgctgccgctgcaga |
| | | cgccgccgcttcctccattgccaagggcggggcttagagct |
| | | caacccagacacctgtaggtgccggaagccgcgaaagtgac |
| | | aagctgctttccagactccacgggcccggctgcttttatgg |
| | | ccctgcttcacagggagaagagtggagcacaggcgaacctc |
| | | ctcagtctgggaggtcactgccccaggacctggacctttta |
| | | gagagctctctcgccatcttttatctcccagagctgccatc |
| | | taacaattgtcaaggaacctcatgtctcacctcaggggcca |
| | | gggtactctctcacttaaccaccctggtcaagtgagcatct |
| | | tctggctggctgtctcccctcactatgaaaaccccaaactt |
| | | ctaccaataacgggatttgggttctgttatgataactgtga |
| | | cacacacacacactcacactctgataaaagagatggaagac |
| | | actaac |
| 6. | VEGF-B | MSPLLRRLLLVALLQLARTQAPVSQFDGPSHQKKVVPWIDV |
| | (mouse) AA | YARATCQPREVVVPLSMELMGNVVKQLVPSCVTVQRCGGCC |
| | Genbank | PDDGLECVPTGQHQVRMQILMIQYPSSQLGEMSLEEHSQCE |
| | Acc No.: | CRPKKKESAVKPDRVAIPHHRPQPRSVPGWDSTPGASSPAD |
| | NM_011697 | IIHPTPAPGSSARLAPSAVNALTPGPAAAAADAAASSIAKG |
| | | GA |
| 7. | VEGF-B₁₆₇ | MSPLLRRLLLVALLQLARTQAPVSQFDGPSHQKKVVPWIDV |
| | (mouse) AA | YARATCQPREVVVPLSMELMGNVVKQLVPSCVTVQRCGGCC |
| | | PDDGLECVPTGQHQVRMQILMIQYPSSQLGEMSLEEHSQCE |
| | | CRPKKKESAVKPDSPRILCPPCTQRRQRPDPRTCRCRCRRR |
| | | RFLHCQGRGLELNPDTCRCRKPRK |
| 8. | VEGF-B₁₈₆ | MSPLLRRLLLVALLQLARTQAPVSQFDGPSHQKKVVPWIDV |
| | (mouse) AA | YARATCQPREVVVPLSMELMGNVVKQLVPSCVTVQRCGGCC |
| | Genbank | PDDGLECVPTGQHQVRMQILMIQYPSSQLGEMSLEEHSQCE |
| | Acc. No.: | CRPKKKESAVKPDRVAIPHHRPQPRSVPGWDSTPGASSPAD |
| | U52820 | IIHPTPAPGSSARLAPSAVNALTPGPAAAAADAAASSIAKG |
| | | GA |

Use of polypeptides comprising VEGF-B sequences modified with conservative substitutions, insertions, and/or deletions, but which still retain the biological activity of VEGF-B is within the scope of the invention. Standard methods can readily be used to generate such polypeptides including site-directed mutagenesis of VEGF-B polynucleotides, or specific enzymatic cleavage and ligation. Similarly, use of peptidomimetic compounds or compounds in which one or more amino acid residues are replaced by a non-naturally-occurring amino acid or an amino acid analog that retains the required aspects of the biological activity of VEGF-B is contemplated.

In addition, variant forms of VEGF-B polypeptides that may result from alternative splicing and naturally-occurring allelic variation of the nucleic acid sequence encoding VEGF-B are useful in the invention. Allelic variants are well known in the art, and represent alternative forms or a nucleic acid sequence that comprise substitution, deletion or addition of one or more nucleotides, but which do not result in any substantial functional alteration of the encoded polypeptide.

Variant forms of VEGF-B can be prepared by targeting non-essential regions of a VEGF-B polypeptide for modification. These non-essential regions are expected to fall outside the strongly-conserved regions of the VEGF/PDGF family of growth factors. In particular, the growth factors of the PDGF/VEGF family, including VEGF-B and the PDGFs, are dimeric, and at least VEGF-A, VEGF-B, VEGF-C, VEGF-D, PlGF, PDGF-A and PDGF-B show complete conservation of eight cysteine residues in the N-terminal domains, i.e. the PDGF/VEGF-like domains. (Olofsson, et al., Proc. Nat'l Acad. Sci. USA, 93:2576-2581 (1996); Joukov, et al., EMBO J., 15:290-298 (1996)). These cysteines are thought to be involved in intra- and inter-molecular disulfide bonding. In addition there are further strongly, but not completely, conserved cysteine residues in the C-terminal domains. Loops 1, 2 and 3 of each subunit, which are formed by intra-molecular disulfide bonding, are involved in binding to the receptors for the PDGF/VEGF family of growth factors. (Andersson, et al., Growth Factors, 12:159-64 (1995)).

These conserved cysteine residues are preferably preserved in any proposed variant form, although there may be exceptions, because receptor-binding VEGF-B analogs are known in which one or more of the cysteines is not conserved. Similarly, the active sites present in loops 1, 2 and 3 also should be preserved. Other regions of the molecule can be expected to be of lesser importance for biological function, and therefore offer suitable targets for modification. Modified polypeptides can readily be tested for their ability to show the biological activity of VEGF-B by routine activity assay such as a VEGFR-1 binding assay or an FATP modulating activity assays based on the examples set forth below. Alignment of VEGF-B sequences from multiple species, to identify conserved and variable residues, provides an indication of residues that are more susceptible to alteration without destroying VEGFR-1 binding activity.

Preferably, where amino acid substitution is used, the substitution is conservative, i.e. an amino acid is replaced by one of similar size and with similar charge properties.

As used herein, the term “conservative substitution” denotes the replacement of an amino acid residue by another, biologically similar residue. Examples of conservative substitutions include the substitution of one hydrophobic residue such as isoleucine, valine, leucine, alanine, cysteine, glycine, phenylalanine, proline, tryptophan, tyrosine, norleucine or methionine for another, or the substitution of one polar or charged residue for another residue with similarly polarity or charge, such as the substitution of arginine for lysine, glutamic acid for aspartic acid, or glutamine for asparagine, and the like. Neutral hydrophilic amino acids that can be substituted for one another include asparagine, glutamine, serine and threonine. The term “conservative substitution” also includes the use of a substituted amino acid in place of an unsubstituted parent amino acid.

Alternatively, conservative amino acids can be grouped as described in Lehninger, (Biochemistry, Second Edition; Worth Publishers, Inc. NY: NY, pp. 71-77 (1975)) as set out in the following:

Non-Polar (Hydrophobic)
A. Aliphatic: A, L, I, V, P,
B. Aromatic: F, W,
C. Sulfur-containing: M,
D. Borderline: G.

Uncharged-Polar
A. Hydroxyl: S, T, Y,
B. Amides: N, Q,
C. Sulthydryl: C,
D. Borderline: G.

Positively Charged (Basic): K, R, H.

Negatively Charged (Acidic): D, E.

VEGF-B protein can be modified, for instance, by glycosylation, amidation, carboxylation, or phosphorylation, or by the creation of acid addition salts, amides, esters, in particular C-terminal esters, and N-acyl derivatives. The proteins also can be modified to create peptide derivatives by forming covalent or noncovalent complexes with other moieties. Covalently bound complexes can be prepared by linking the chemical moieties to functional groups on the side chains of amino acids comprising the peptides, or at the N- or C-terminus.

VEGF-B proteins can be conjugated to a reporter group, including, but not limited to a radiolabel, a fluorescent label, an enzyme (e.g., that catalyzes a calorimetric or fluorometric reaction), a substrate, a solid matrix, or a carrier (e.g., biotin or avidin).

Examples of VEGF-B analogs are described in WO 98/28621 and in Olofsson, et al., Proc. Nat'l. Acad. Sci. USA, 95:11709-11714 (1998), both incorporated herein by reference.

VEGF-B polypeptides are preferably produced by expression of DNA sequences that encode them such as DNAs that correspond to, or that hybridize under stringent conditions with the complements of SEQ ID NOS: 1 and 2. Suitable hybridization conditions include, for example, 50% formamide, 5×SSPE buffer, 5× Denhardts solution, 0.5% SDS and 100 μg/ml of salmon sperm DNA at 42° C. overnight, followed by washing 2×30 minutes in 2×SSC at 55° C. Such hybridization conditions are applicable to any polynucleotide encoding one or more of the growth factors of the present invention.

The invention is also directed to an isolated and/or purified DNA that corresponds to, or that hybridizes under stringent conditions with, any one of the foregoing DNA sequences.

The VEGF-B for use according to the present invention can be used in the form of a protein dimer comprising VEGF-B protein, particularly a disulfide-linked dimer. VEGF-B dimers may comprise VEGF-B polypeptides of identical sequence, of different VEGF-B isoforms, or other heterogeneous VEGF-B molecules. The protein dimers for use according to the present invention include both homodimers of VEGF-B and heterodimers of VEGF-B and VEGF polypeptides, as well as other VEGF family growth factors including, but not limited to placental growth factor (PlGF), which are capable of binding to VEGFR-1 (flt-1). The VEGF-B of the present invention also includes VEGF-B polypeptides that have been engineered to contain an N-glycosylation site such as those described in Jeltsch, et al., WO 02/07514, which is incorporated herein in its entirety.

The term “vector” refers to a nucleic acid molecule amplification, replication, and/or expression vehicle in the form of a plasmid or viral DNA system where the plasmid or viral DNA may be functional with bacterial, yeast, invertebrate, and/or mammalian host cells. The vector may remain independent of host cell genomic DNA or may integrate in whole or in part with the genomic DNA. The vector will contain all necessary elements so as to be functional in any host cell it is compatible with. Such elements are set forth below.

A. Preparation of DNA Encoding VEGF-B Polypeptides

A nucleic acid molecule encoding a VEGF-B polypeptide can readily be obtained in a variety of ways, including, without limitation, chemical synthesis, cDNA or genomic library screening, expression library screening, and/or PCR amplification of cDNA. These methods and others useful for isolating such DNA are set forth, for example, by Sambrook, et al., “Molecular Cloning: A Laboratory Manual,” Cold Spring Harbor Laboratory Press, Cold Spring Harbor, N.Y. (1989), by Ausubel, et al., eds., “Current Protocols In Molecular Biology,” Current Protocols Press (1994), and by Berger and Kimmel, “Methods In Enzymology: Guide To Molecular Cloning Techniques,” vol. 152, Academic Press, Inc., San Diego, Calif. (1987). Preferred nucleic acid sequences encoding VEGF-B are mammalian sequences. Most preferred nucleic acid sequences encoding VEGF-B are human and other primates.

Chemical synthesis of a VEGF-B nucleic acid molecule can be accomplished using methods well known in the art, such as those set forth by Engels, et al., Angew. Chem. Intl. Ed., 28:716-734 (1989). These methods include, inter alia, the phosphotriester, phosphoramidite and H-phosphonate methods of nucleic acid synthesis. Typically, the nucleic acid molecule encoding the full length VEGF-B polypeptide will be several hundred base pairs (bp) or nucleotides in length. Nucleic acids larger than about 100 nucleotides in length can be synthesized as several fragments, each fragment being up to about 100 nucleotides in length. The fragments can then be ligated together, as described below, to form a full length nucleic acid encoding the VEGF-B polypeptide. A preferred method is polymer-supported synthesis using standard phosphoramidite chemistry.

Alternatively, the nucleic acid encoding a VEGF-B polypeptide may be obtained by screening an appropriate cDNA library prepared from one or more tissue source(s) that express the polypeptide, or a genomic library from any subspecies. The source of the genomic library may be any tissue or tissues from any mammalian or other species believed to harbor a gene encoding VEGF-B or a VEGF-B homologue.

The library can be screened for the presence of the VEGF-B cDNA/gene using one or more nucleic acid probes (oligonucleotides, cDNA or genomic DNA fragments that possess an acceptable level of homology to the VEGF-B or VEGF-B homologue cDNA or gene to be cloned) that will hybridize selectively with VEGF-B or VEGF-B homologue cDNA(s) or gene(s) that is(are) present in the library. The probes preferably are complementary to or encode a small region of the VEGF-B DNA sequence from the same or a similar species as the species from which the library was prepared. Alternatively, the probes may be degenerate, as discussed below.

Where DNA fragments (such as cDNAs) are used as probes, typical hybridization conditions are those for example as set forth in Ausubel, et al., eds., supra. After hybridization, the blot containing the library is washed at a suitable stringency, depending on several factors such as probe size, expected homology of probe to clone, type of library being screened, number of clones being screened, and the like. Examples of stringent washing solutions (which are usually low in ionic strength and are used at relatively high temperatures) are as follows. One such stringent wash is 0.015 M NaCl, 0.005 M NaCitrate and 0.1 percent SDS at 55-65° C. Another such stringent buffer is 1 mM Na₂ EDTA, 40 mM NaHPO₄, pH 7.2, and 1% SDS at about 40-50° C. One other stringent wash is 0.2×SSC and 0.1% SDS at about 50-65° C. Such hybridization conditions are applicable to any polynucleotide encoding one or more of the growth factors of the present invention.

Another suitable method for obtaining a nucleic acid encoding a VEGF-B polypeptide is the polymerase chain reaction (PCR). In this method, poly(A)+RNA or total RNA is extracted from a tissue that expresses VEGF-B. cDNA is then prepared from the RNA using the enzyme reverse transcriptase. Two primers typically complementary to two separate regions of the VEGF-B cDNA (oligonucleotides) are then added to the cDNA along with a polymerase such as Taq polymerase, and the polymerase amplifies the cDNA region between the two primers.

B. Preparation of a Vector for VEGF-B Expression

After cloning, the cDNA or gene encoding a VEGF-B polypeptide or fragment thereof has been isolated, it is preferably inserted into an amplification and/or expression vector in order to increase the copy number of the gene and/or to express the polypeptide in a suitable host cell and/or to transform cells in a target organism (to express VEGF-B in vivo). Numerous commercially available vectors are suitable, though “custom made” vectors may be used as well. The vector is selected to be functional in a particular host cell or host tissue (i.e., the vector is compatible with the host cell machinery such that amplification of the VEGF-B gene and/or expression of the gene can occur). The VEGF-B polypeptide or fragment thereof may be amplified/expressed in prokaryotic, yeast, insect (baculovirus systems) and/or eukaryotic host cells. Selection of the host cell will depend at least in part on whether the VEGF-B polypeptide or fragment thereof is to be glycosylated. If so, yeast, insect, or mammalian host cells are preferable; yeast cells will glycosylate the polypeptide if a glycosylation site is present on the VEGF-B amino acid sequence.

Typically, the vectors used in any of the host cells will a contain 5′ flanking sequence and other regulatory elements as well such as enhancer(s), an origin of replication element, a transcriptional termination element, a complete intron sequence containing a donor and acceptor splice site, a signal peptide sequence, a ribosome binding site element, a polyadenylation sequence, a polylinker region for inserting the nucleic acid encoding the polypeptide to be expressed, and a selectable marker element. Optionally, the vector may contain a “tag” sequence, i.e., an oligonucleotide sequence located at the 5′ or 3′ end of the VEGF-B coding sequence that encodes polyHis (such as hexaHis) or another small immunogenic sequence. This tag will be expressed along with the protein, and can serve as an affinity tag for purification of the VEGF-B polypeptide from the host cell. Optionally, the tag can subsequently be removed from the purified VEGF-B polypeptide by various means such as using a selected peptidase for example.

The vector/expression construct may optionally contain elements such as a 5′ flanking sequence, an origin of replication, a transcription termination sequence, a selectable marker sequence, a ribosome binding site, a signal sequence, and one or more intron sequences. The 5′ flanking sequence may be homologous (i.e., from the same species and/or strain as the host cell), heterologous (i.e., from a species other than the host cell species or strain), hybrid (i.e., a combination of p5′ flanking sequences from more than one source), synthetic, or it may be the native VEGF-B 5′ flanking sequence. As such, the source of the 5′ flanking sequence may be any unicellular prokaryotic or eukaryotic organism, any vertebrate or invertebrate organism, or any plant, provided that the 5′ flanking sequence is functional in, and can be activated by, the host cell machinery.

An origin of replication is typically a part of commercial prokaryotic expression vectors, and aids in the amplification of the vector in a host cell. Amplification of the vector to a certain copy number can, in some cases, may be important for optimal expression of the VEGF-B polypeptide. If the vector of choice does not contain an origin of replication site, one may be chemically synthesized based on a known sequence, and ligated into the vector.

A transcription termination element is typically located 3′ to the end of the VEGF-B polypeptide coding sequence and serves to terminate transcription of the VEGF-B polypeptide. Usually, the transcription termination element in prokaryotic cells is a G-C rich fragment followed by a poly T sequence. Such elements can be cloned from a library, purchased commercially as part of a vector, and readily synthesized.

Selectable marker genes encode proteins necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells, (b) complement auxotrophic deficiencies of the cell; or (c) supply critical nutrients not available from complex media.

A ribosome binding element, commonly called the Shine-Dalgarno sequence (prokaryotes) or the Kozak sequence (eukaryotes), is necessary for translation initiation of mRNA. The element is typically located 3′ to the promoter and 5′ to the coding sequence of the polypeptide to be synthesized. The Shine-Dalgarno sequence is varied but is typically a polypurine (i.e., having a high A-G content). Many Shine-Dalgarno sequences have been identified, each of which can be readily synthesized using methods set forth above.

All of the elements set forth above, as well as others useful in this invention, are well known to the skilled artisan and are described, for example, in Sambrook, et al., “Molecular Cloning: A Laboratory Manual,” Cold Spring Harbor Laboratory Press, Cold Spring Harbor, N.Y. (1989) and Berger, et al., eds., “Guide To Molecular Cloning Techniques,” Academic Press, Inc., San Diego, Calif (1987].

For those embodiments of the invention where the recombinant VEGF-B is to be secreted, a signal sequence is preferably included to direct secretion from the cell where it is synthesized. Typically, the signal sequence is positioned in the coding region of the transgene towards or at the 5′ end of the coding region. Many signal sequences have been identified, and any of them that are functional in the transgenic tissue may be used in conjunction with the transgene. Therefore, the signal sequence may be homologous or heterologous to the transgene, and may be homologous or heterologous to the transgenic mammal. Additionally, the signal sequence may be chemically synthesized using methods set forth above. However, for purposes herein, preferred signal sequences are those that occur naturally with the transgene (i.e., are homologous to the transgene).

In many cases, gene transcription is increased by the presence of one or more introns on the vector. The intron may be naturally-occurring within the transgene sequence, especially where the transgene is a full length or a fragment of a genomic DNA sequence. Where the intron is not naturally-occurring within the DNA sequence (as for most cDNAs), the intron(s) may be obtained from another source. The intron may be homologous or heterologous to the transgene and/or to the transgenic mammal. The position of the intron with respect to the promoter and the transgene is important, as the intron must be transcribed to be effective. As such, where the transgene is a cDNA sequence, the preferred position for the intron is 3′ to the transcription start site, and 5′ to the polyA transcription termination sequence. Preferably for cDNA transgenes, the intron will be located on one side or the other (i.e., 5′ or 3′) of the transgene sequence such that it does not interrupt the transgene sequence. Any intron from any source, including any viral, prokaryotic and eukaryotic (plant or animal) organisms, may be used to express VEGF-B, provided that it is compatible with the host cell(s) into which it is inserted. Also included herein are synthetic introns. Optionally, more than one intron may be used in the vector.

Preferred vectors for recombinant expression of VEGF-B protein are those that are compatible with bacterial, insect, and mammalian host cells. Such vectors include, inter alia, pCRII (Invitrogen Company, San Diego, Calif.), pBSII (Stratagene Company, La Jolla, Calif.), and pETL (BlueBacII; Invitrogen).

After the vector has been constructed and a VEGF-B nucleic acid has been inserted into the proper site of the vector, the completed vector may be inserted into a suitable host cell for amplification and/or VEGF-B polypeptide expression. The host cells typically used include, without limitation: Prokaryotic cells such as gram negative or gram positive cells, i.e., any strain of E. coli, Bacillus, Streptomyces, Saccharomyces, Salmonella, and the like; eukaryotic cells such as CHO (Chinese hamster ovary) cells, human kidney 293 cells, COS-7 cells; insect cells such as Sf4, Sf5, Sf9, and Sf21 and High 5 (all from the Invitrogen Company, San Diego, Calif.); and various yeast cells such as Saccharomyces and Pichia.

Insertion (also referred to as “transformation” or “transfection”) of the vector into the selected host cell may be accomplished using such methods as calcium chloride, electroporation, microinjection, lipofection or the DEAE-dextran method. The method selected will in part be a function of the type of host cell to be used. These methods and other suitable methods are well known to the skilled artisan, and are set forth, for example, in Sambrook, et al., supra.

The host cells containing the vector (i.e., transformed or transfected) may be cultured using standard media well known to the skilled artisan. The media will usually contain all nutrients necessary for the growth and survival of the cells. Suitable media for culturing E. coli cells are for example, Luria Broth (LB) and/or Terrific Broth (TB). Suitable media for culturing eukaryotic cells are RPMI 1640, MEM, DMEM, all of which may be supplemented with serum and/or growth factors as required by the particular cell line being cultured. A suitable medium for insect cultures is Grace's medium supplemented with yeastolate, lactalbumin hydrolysate, and/or fetal calf serum as necessary.

Typically, an antibiotic or other compound useful for selective growth of the transformed cells only is added as a supplement to the media. The compound to be used will be dictated by the selectable marker element present on the plasmid with which the host cell was transformed. For example, where the selectable marker element is kanamycin resistance, the compound added to the culture medium will be kanamycin.

The amount of VEGF-B polypeptide produced in the host cell can be evaluated using standard methods known in the art. Such methods include, without limitation, Western blot analysis, SDS-polyacrylamide gel electrophoresis, non-denaturing gel electrophoresis, HPLC separation, immunoprecipitation, and/or activity assays such as VEGFR-1 binding assays or cell stimulation assays.

C. Purification of VEGF-B Polypeptides

VEGF-B polypeptides are preferably expressed and purified as described in U.S. Pat. No. 6,331,301, incorporated herein by reference.

If the VEGF-B polypeptide is designed to be secreted from the host cells, the majority of the polypeptide will likely be found in the cell culture medium. If, however, the VEGF-B polypeptide is not secreted from the host cells, it will be present in the cytoplasm (for eukaryotic, gram positive bacteria, and insect host cells) or in the periplasm (for gram negative bacteria host cells).

For intracellular VEGF-B, the host cells are first disrupted mechanically or osmotically to release the cytoplasmic contents into a buffered solution. The polypeptide is then isolated from this solution.

Purification of VEGF-B polypeptide from solution can be accomplished using a variety of techniques. If the polypeptide has been synthesized such that it contains a tag such as Hexahistidine (VEGF-B/hexaHis) or other small peptide at either its carboxyl or amino terminus, it may essentially be purified in a one-step process by passing the solution through an affinity column where the column matrix has a high affinity for the tag or for the polypeptide directly (i.e., a monoclonal antibody specifically recognizing VEGF-B). For example, polyhistidine binds with great affinity and specificity to nickel, thus an affinity column of nickel (such as the Qiagen nickel columns) can be used for purification of VEGF-B/polyHis. (See, for example, Ausubel, et al., eds., “Current Protocols In Molecular Biology,” Section 10.11.8, John Wiley & Sons, New York (1993)).

The strong affinity of VEGF-B for its receptor VEGFR-1 permits affinity purification of VEGF-B using an affinity matrix comprising VEGFR-1 extracellular domain. In addition, where the VEGF-B polypeptide has no tag and no antibodies are available, other well known procedures for purification can be used. Such procedures include, without limitation, ion exchange chromatography, molecular sieve chromatography, HPLC, native gel electrophoresis in combination with gel elution, and preparative isoelectric focusing (“Isoprime” machine/technique, Hoefer Scientific). In some cases, two or more of these techniques may be combined to achieve increased purity. Preferred methods for purification include polyHistidine tagging and ion exchange chromatography in combination with preparative isoelectric focusing.

VEGF-B polypeptide found in the periplasmic space of the bacteria or the cytoplasm of eukaryotic cells, the contents of the periplasm or cytoplasm, including inclusion bodies (bacteria) if the processed polypeptide has formed such complexes, can be extracted from the host cell using any standard technique known to the skilled artisan. For example, the host cells can be lysed to release the contents of the periplasm by French press, homogenization, and/or sonication. The homogenate can then be centrifuged.

If the VEGF-B polypeptide has formed inclusion bodies in the periplasm, the inclusion bodies can often bind to the inner and/or outer cellular membranes and thus will be found primarily in the pellet material after centrifugation. The pellet material can then be treated with a chaotropic agent such as guanidine or urea to release, break apart, and solubilize the inclusion bodies. The VEGF-B polypeptide in its now soluble form can then be analyzed using gel electrophoresis, immunoprecipitation or the like. If it is desired to isolate the VEGF-B polypeptide, isolation may be accomplished using standard methods such as those set forth below and in Marston, et al., Meth. Enz., 182:264-275 (1990).

If VEGF-B polypeptide inclusion bodies are not formed to a significant degree in the periplasm of the host cell, the VEGF-B o polypeptide will be found primarily in the supernatant after centrifugation of the cell homogenate, and the VEGF-B polypeptide can be isolated from the supernatant using methods such as those set forth below.

In those situations where it is preferable to partially or completely isolate the VEGF-B polypeptide, purification can be accomplished using standard methods well known to the skilled artisan. Such methods include, without limitation, separation by electrophoresis followed by electroelution, various types of chromatography (immunoaffinity, molecular sieve, and/or ion exchange), and/or high pressure liquid chromatography. In some cases, it may be preferable to use more than one of these methods for complete purification.

Anti-VEGF-B Therapeutic Compounds

Anti-VEGF-B therapies, as discussed below, include but are not limited to antibody, aptamer, antisense and interference RNA techniques and therapies. These agents are contemplated for numerous therapeutic contexts described herein, such as therapies directed to reducing lipid accumulation, and stimulating glucose metabolism and other parameters associated with metabolic syndrome in a mammalian subject.

A. Therapeutic Anti-VEGF-B Antibodies

Anti-VEGF-B antibodies as described in U.S. Pat. No. 6,331,301 are also contemplated for use in practicing the present invention. Such antibodies can be used for VEGF-B purification, or therapeutically where inhibition of VEGF-B is desired. See also WO 2005/087812, WO 2005/087808, U.S. Patent Application Publication No. 2005-0282233, U.S. Patent Application Publication No. 2006-0030000, U.S. Patent Application Publication No. 2008-0260729, and Leonard et al. (2008) Journal of Molecular Biology 384:1203-1217, the disclosures of which are incorporated herein by reference in their entireties.

Polyclonal or monoclonal therapeutic anti-VEGF-B antibodies useful in practicing this invention may be prepared in laboratory animals or by recombinant DNA techniques using the following methods. Polyclonal antibodies to the VEGF-B molecule or a fragment thereof containing the target amino acid sequence generally are raised in animals by multiple subcutaneous (sc) or intraperitoneal (ip) injections of the VEGF-B molecule in combination with an adjuvant such as Freund's adjuvant (complete or incomplete). To enhance immunogenicity, it may be useful to first conjugate the VEGF-B molecule or a fragment containing the target amino acid sequence of to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOC₁, or R¹ N═C═NR, where R and R¹ are different alkyl groups. Alternatively, VEGF-B-immunogenic conjugates can be produced recombinantly as fusion proteins.

Animals are immunized against the immunogenic VEGF-B conjugates or derivatives (such as a fragment containing the target amino acid sequence) by combining about 1 mg or about 1 microgram of conjugate (for rabbits or mice, respectively) with about 3 volumes of Freund's complete adjuvant and injecting the solution intradermally at multiple sites. Approximately 7 to 14 days later, animals are bled and the serum is assayed for anti-VEGF-B titer. Animals are boosted with antigen repeatedly until the titer plateaus. Preferably, the animal is boosted with the same VEGF-B molecule or fragment thereof as was used for the initial immunization, but conjugated to a different protein and/or through a different cross-linking agent. In addition, aggregating agents such as alum are used in the injections to enhance the immune response.

Monoclonal antibodies may be prepared by recovering spleen cells from immunized animals and immortalizing the cells in conventional fashion, e.g. by fusion with myeloma cells. The clones are then screened for those expressing the desired antibody. In some embodiments, the monoclonal antibody is specific for VEGF-B. In other embodiments, the monoclonal antibody cross-reacts with other VEGF/PDGF family members.

Preparation of antibodies using recombinant DNA methods, such as the phagemid display method, may be accomplished using commercially available kits, as for example, the Recombinant Phagemid Antibody System available from Pharmacia (Uppsala, Sweden), or the SurfZAP™ phage display system (Stratagene Inc., La Jolla, Calif.).

Preferably, antibodies for administration to humans, although prepared in a laboratory animal such as a mouse, will be “humanized”, or chimeric, i.e. made to be compatible with the human immune system such that a human patient will not develop an immune response to the antibody. Even more preferably, human antibodies which can now be prepared using methods such as those described for example, in Lonberg, et al., Nature Genetics, 7:13-21 (1994) are preferred for therapeutic administration to patients.

1. Humanization of Anti-VEGF-B Monoclonal Antibodies

VEGF-B-neutralizing antibodies comprise one class of therapeutics useful as VEGF-B antagonists. Following are protocols to improve the utility of anti-VEGF-B monoclonal antibodies as therapeutics in humans, by “humanizing” the monoclonal antibodies to improve their serum half-life and render them less immunogenic in human hosts (i.e., to prevent human antibody response to non-human anti-VEGF-B antibodies).

The principles of humanization have been described in the literature and are facilitated by the modular arrangement of antibody proteins. To minimize the possibility of binding complement, a humanized antibody of the IgG₄ isotype is preferred.

For example, a level of humanization is achieved by generating chimeric antibodies comprising the variable domains of non-human antibody proteins of interest, such as the anti-VEGF-B monoclonal antibodies described herein, with the constant domains of human antibody molecules. (See, e.g., Morrison and Oi, Adv. Immunol., 44:65-92 (1989)). The variable domains of VEGF-B neutralizing anti-VEGF-B antibodies are cloned from the genomic DNA of a B-cell hybridoma or from cDNA generated from mRNA isolated from the hybridoma of interest. The V region gene fragments are linked to exons encoding human antibody constant domains, and the resultant construct is expressed in suitable mammalian host cells (e.g., myeloma or CHO cells).

To achieve an even greater level of humanization, only those portions of the variable region gene fragments that encode antigen-binding complementarity determining regions (“CDR”) of the non-human monoclonal antibody genes are cloned into human antibody sequences. (See, e.g., Jones, et al., Nature, 321:522-525 (1986); Riechmann, et al., Nature, 332:323-327 (1988); Verhoeyen, et al., Science, 239:1534-36 (1988); and Tempest, et al., Bio/Technology, 9:266-71 (1991)). If necessary, the beta-sheet framework of the human antibody surrounding the CDR3 regions also is modified to more closely mirror the three dimensional structure of the antigen-binding domain of the original monoclonal antibody. (See, Kettleborough, et al., Protein Engin., 4:773-783 (1991); and Foote, et al., J. Mol. Biol., 224:487-499 (1992)).

In an alternative approach, the surface of a non-human monoclonal antibody of interest is humanized by altering selected surface residues of the non-human antibody, e.g., by site-directed mutagenesis, while retaining all of the interior and contacting residues of the non-human antibody. See Padlan, Molecular Immunol., 28(4/5):489-98 (1991).

The foregoing approaches are employed using VEGF-B-neutralizing anti-VEGF-B monoclonal antibodies and the hybridomas that produce them to generate humanized VEGF-B-neutralizing antibodies useful as therapeutics to treat or palliate conditions wherein VEGF-B expression is detrimental.

2. Human VEGF-B-Neutralizing Antibodies from Phage Display

Human VEGF-B-neutralizing antibodies are generated by phage display techniques such as those described in Aujame, et al., Human Antibodies, 8(4):155-168 (1997); Hoogenboom, TIBTECH, 15:62-70 (1997); and Rader, et al., Curr. Opin. Biotechnol., 8:503-508 (1997), all of which are incorporated by reference. For example, antibody variable regions in the form of Fab fragments or linked single chain Fv fragments are fused to the amino terminus of filamentous phage minor coat protein pIII. Expression of the fusion protein and incorporation thereof into the mature phage coat results in phage particles that present an antibody on their surface and contain the genetic material encoding the antibody. A phage library comprising such constructs is expressed in bacteria, and the library is panned (screened) for VEGF-B-specific phage-antibodies using labeled or immobilized VEGF-B respectively as antigen-probe.

3. Human VEGF-B-Neutralizing Antibodies from Transgenic Mice

Human VEGF-B-neutralizing antibodies are generated in transgenic mice essentially as described in Bruggemann and Neuberger, Immunol. Today, 17(8):391-97 (1996) and Bruggemann and Taussig, Curr. Opin. Biotechnol., 8:455-58 (1997). Transgenic mice carrying human V-gene segments in germline configuration and that express these transgenes in their lymphoid tissue are immunized with VEGF-B composition using conventional immunization protocols. Hybridomas are generated using B cells from the immunized mice using conventional protocols and screened to identify hybridomas secreting anti-VEGF-B human antibodies (e.g., as described above).

4. Bispecific Antibodies

Bispecific antibodies that specifically bind to one protein (e.g., VEGF-B) and that specifically bind to other antigens relevant to pathology and/or treatment are produced, isolated, and tested using standard procedures that have been described in the literature. See, e.g., Pluckthun & Pack, Immunotechnology, 3:83-105 (1997); Carter, et al., J. Hematotherapy, 4: 463-470 (1995); Renner & Pfreundschuh, Immunological Reviews, 1995, No. 145, pp. 179-209; Pfreundschuh U.S. Pat. No. 5,643,759; Segal, et al., J. Hematotherapy, 4: 377-382 (1995); Segal, et al., Immunobiology, 185: 390-402 (1992); and Bolhuis, et al., Cancer Immunol. Immunother., 34: 1-8 (1991), all of which are incorporated herein by reference in their entireties. For example, in one embodiment, bispecific antibodies that specifically bind to VEGF-B and specifically bind to VEGF-A or PlGF is contemplated.

B. Anti-VEGFR-1 Antibodies

Anti-VEGFR-1 antibodies that inhibit binding to and activation of VEGF-B are also contemplated as another inhibitor of VEGF-B that is useful for practicing the invention. Suitable antibodies include Avastin, mAb 6.12, and MF1. Likewise, monoclonal, polyclonal, human, humanized and bispecific antibodies are also contemplated.

C. VEGFR-1 Antagonists

VEGFR-1 antagonists that inhibit binding to and activation of VEGF-B are also contemplated as another inhibitor of VEGF-B that is useful for practicing the invention. Suitable VEGFR-1 antagonists include Sutent, SU5416, and those disclosed in WO 2005/087808 and US Patent Publication No. 2006-0030000, the disclosure of which are incorporated herein by reference in their entireties.

D. Anti-VEGF-B Aptamers

Recent advances in the field of combinatorial sciences have identified short polymer sequences with high affinity and specificity to a given target. For example, SELEX technology has been used to identify DNA and RNA aptamers with binding properties that rival mammalian antibodies, the field of immunology has generated and isolated antibodies or antibody fragments which bind to a myriad of compounds and phage display has been utilized to discover new peptide sequences with very favorable binding properties. Based on the success of these molecular evolution techniques, ligands can be created which bind to VEGF-B. Curiously, in each case, a loop structure is often involved with providing the desired binding attributes as in the case of: aptamers which often utilize hairpin loops created from short regions without complimentary base pairing, naturally derived antibodies that utilize combinatorial arrangement of looped hyper-variable regions and new phage display libraries utilizing cyclic peptides that have shown improved results when compare to linear peptide phage display results. Thus, sufficient evidence has been generated to suggest that high affinity ligands can be created and identified by combinatorial molecular evolution techniques. For the present invention, molecular evolution techniques can be used to isolate ligands specific for VEGF-B, to be used in a manner analogous to that discussed above for anti-VEGF-B antibodies. For more on aptamers, see generally, Gold, L., Singer, B., He, Y. Y., Brody. E., “Aptamers As Therapeutic And Diagnostic Agents,” J. Biotechnol. 74:5-13 (2000).

E. Anti-Sense Molecules and Therapy

Another class of VEGF-B inhibitors useful in the present invention is isolated antisense nucleic acid molecules that can hybridize to, or are complementary to, the nucleic acid molecule comprising the VEGF-B nucleotide sequence, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). (See, for example, Uhlmann, et al. Antisense oligonucleotides: A new therapeutic principle. Chemical Reviews 1990, 90: 543-584; Crooke, et al. “Antisense Research and Applications”, CRC Press (1993); Mesmaekar, et al. “Antisense oligonucleotides,” Acc. Chem. Res. 1995, 28: 366-374; Stein. “The experimental use of antisense oligonucleotides: a guide for the perplexed.” J. Clin. Invest. 2001, 108, 641-644, and U.S. Pat. Nos. 6,117,992; 6,127,121; 6,235,887; 6,232,463; 6,579,704; 5,596,091; 6,031,086 and 6,117,992, the disclosures of which are incorporated herein by reference in their entireties). In specific aspects, antisense nucleic acid molecules are provided that comprise a sequence complementary to at least about 10, 25, 50, 100, 250 or 500 nucleotides or an entire VEGF-B coding strand, or to only a portion thereof. Nucleic acid molecules encoding fragments, homologs, derivatives and analogs of VEGF-B antisense nucleic acids complementary to a VEGF-B nucleic acid sequence are additionally provided.

In one embodiment, an antisense nucleic acid molecule is antisense to a “coding region” of the coding strand of a nucleotide sequence encoding a VEGF-B protein. The term “coding region” refers to the region of the nucleotide sequence comprising codons that are translated into amino acid residues. In another embodiment, the antisense nucleic acid molecule is antisense to a “conceding region” of the coding strand of a nucleotide sequence encoding the VEGF-B protein. The term “conceding region” refers to 5′ and 3′ sequences that flank the coding region and that are not translated into amino acids (i.e., also referred to as 5′ and 3′ untranslated regions).

Given the coding strand sequences encoding the VEGF-B protein disclosed herein, antisense nucleic acids of the invention can be designed according to the rules of Watson and Crick or Hoogsteen base pairing. The antisense nucleic acid molecule can be complementary to the entire coding region of VEGF-B mRNA, but more preferably is an oligonucleotide that is antisense to only a portion of the coding or noncoding region of VEGF-B mRNA. For example, the antisense oligonucleotide can be complementary to the region surrounding the translation start site of VEGF-B mRNA. An antisense oligonucleotide can be, for example, about 5, 10, 15, 20, 25, 30, 35, 40, 45, or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridin-e, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiour-acil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following section).

The antisense nucleic acid molecules of the invention are typically administered to a subject or generated in situ such that they hybridize with or bind to cellular mRNA and/or genomic DNA encoding VEGF-B to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an alpha-anomeric nucleic acid molecule. An alpha-anomeric nucleic acid molecule forms specific double-stranded hybrids with complementary RNA in which, contrary to the usual alpha-units, the strands run parallel to each other. See, e.g., Gaultier, et al., Nucl. Acids Res., 15:6625-6641 (1987). The antisense nucleic acid molecule can also comprise a 2′-o-methylribonucleotide (see, e.g., Inoue, et al. Nucl. Acids Res., 15:6131-6148 (1987)) or a chimeric RNA-DNA analogue (see, e.g., Inoue, et al., FEBS Lett., 215:327-330 (1987)).

Production and delivery of antisense molecules are facilitated by providing a vector comprising an anti-sense nucleotide sequence complementary to at least a part of the VEGF-B DNA sequence. According to a yet further aspect of the invention such a vector comprising an anti-sense sequence may be used to inhibit, or at least mitigate, VEGF-B expression. The use of a vector of this type to inhibit VEGF-B expression is favored in instances where VEGF-B expression is associated with a particular disease state.

F. Anti-VEGF-B RNA Interference

Use of RNA Interference to inactivate or modulate VEGF-B expression is also contemplated as part of this invention. RNA interference is described in U.S. Patent Appl. No. 2002-0162126, and Hannon, G., J. Nature, 11:418:244-51 (2002). “RNA interference,” “post-transcriptional gene silencing,” “quelling”—these terms have all been used to describe similar effects that result from the over-expression or misexpression of transgenes, or from the deliberate 5′ introduction of double-stranded RNA into cells (reviewed in Fire, A., Trends Genet 15:358-363 (1999); Sharp, P. A., Genes Dev., 13:139-141 (1999); Hunter, C., Curr. Biol., 9:R440-R442 (1999); Baulcombe, D. C., Curr. Biol. 9:R599-R601 (1999); Vaucheret, et al. Plant J. 16:651-659 (1998), all incorporated by reference. RNA interference, commonly referred to as RNAi, offers a way of specifically and potently inactivating a cloned gene.

RNAi is a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of as few as 21 to 22 base pairs in length. Accordingly, RNAi may be effected by introduction or expression of relatively short homologous dsRNAs. Indeed the use of relatively short homologous dsRNAs may have certain advantages as discussed below.

Mammalian cells have at least two pathways that are affected by double-stranded RNA (dsRNA). In the RNAi (sequence-specific) pathway, the initiating dsRNA is first broken into short interfering (si) RNAs, as described above. The siRNAs have sense and antisense strands of about 21 nucleotides that form approximately 19 nucleotide si RNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length.

The nonspecific effects occur because dsRNA activates two enzymes: PKR, which in its active form phosphorylates the translation initiation factor eIF2 to shut down all protein synthesis, and 2′,5′ oligoadenylate synthetase (2′,5′-AS), which synthesizes a molecule that activates RNase L, a nonspecific enzyme that targets all mRNAs. The nonspecific pathway may represents a host response to stress or viral infection, and, in general, the effects of the nonspecific pathway are preferably minimized. Significantly, longer dsRNAs appear to be required to induce the nonspecific pathway and, accordingly, dsRNAs shorter than about 30 bases pairs are preferred to effect gene repression by RNAi (see Hunter et al. (1975) J. Biol. Chem. 250: 409-17; Manche et al. (1992) Mol. Cell Biol. 12: 5239-48; Minks et al. (1979) J. Biol. Chem. 254: 10180-3; and Elbashir et al. (2001) Nature 411: 494-8). RNAi has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see Bass (2001) Nature 411: 428-9). In mammalian cells, siRNAs are effective at concentrations that are several orders of magnitude below the concentrations typically used in antisense experiments (Elbashir et al. (2001) Nature 411: 494-8).

The double stranded oligonucleotides used to affect RNAi are preferably less than 30 base pairs in length and, more preferably, comprise about 25, 24, 23, 22, 21, 20, 19, 18 or 17 base. pairs of ribonucleic acid. Optionally the dsRNA oligonucleotides may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashi et al. (2001) Nature 411: 494-8). Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernible to the skilled artisan. Exemplary dsRNAs may be synthesized chemically or produced in vitro or in vivo using appropriate expression vectors. Exemplary synthetic RNAs include 21 nucleotide RNAs chemically synthesized using methods known in the art. Synthetic oligonucleotides are preferably deprotected and gel-purified using methods known in the art (see e.g. Elbashir et al. (2001) Genes Dev. 15: 188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence. Any of the above RNA species will be designed to include a portion of nucleic acid sequence represented in a VEGF-B nucleic acid.

Similar to the antisense molecules discussed above, RNAi suitable for the present invention may also be made VEGF-B isoform-specific.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference.

Although mRNAs are generally thought of as linear molecules containing the information for directing protein synthesis within the sequence of ribonucleotides, most mRNAs have been shown to contain a number of secondary and tertiary structures. Secondary structure elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see e.g. Jaeger et al. (1989) Proc. Natl. Acad. Sci. USA 86:7706 (1989); and Turner et al. (1988) Annu. Rev. Biophys. Biophys. Chem. 17:167). The rules are useful in identification of RNA structural elements and, in particular, for identifying single stranded RNA regions which that represent preferred segments of the mRNA to target for silencing RNAi, ribozyme or antisense technologies. Accordingly, preferred segments of the mRNA target can be identified for design of the RNAi mediating dsRNA oligonucleotides.

The dsRNA oligonucleotides may be introduced into the cell by transfection with a heterologous target gene using carrier compositions such as liposomes, which are known in the art—e.g. Lipofectamine 2000 (Life Technologies) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP-encoding pAD3 (Kehlenback et al., 1998, J. Cell Biol. 141: 863-74). The effectiveness of the RNAi may be assessed by any of a number of assays following introduction of the dsRNAs. These include Western blot analysis using antibodies which recognize the VEGF-B gene product following sufficient time for turnover of the endogenous pool after new protein synthesis is repressed, reverse transcriptase polymerase chain reaction and Northern blot analysis to determine the level of existing VEGF-B target mRNA.

Further compositions, methods and applications of RNAi technology are provided in U.S. Pat. Nos. 6,278,039, 5,723,750 and 5,244,805, which are incorporated herein by reference.

Therapeutic Uses for the Anti-VEGF-B Compounds

The present invention provides for both prophylactic and therapeutic methods of treating subjects (e.g., humans or other animals). In one aspect, the invention provides preventing or treating a disease or a disorder in a subject through prophylactic or therapeutic methods.

Administration of a therapeutic agent in a prophylactic method can occur prior to the manifestation of symptoms of an undesired disease or disorder, such that the disease or disorder is prevented or, alternatively, delayed in its progression.

As used herein, the terms “treating” or “treatment” includes the application or administration of a therapeutic agent to a subject who is afflicted with a disease, a symptom of disease or a predisposition toward an undesired disease or disorder, with the goal of curing, healing, alleviating, relieving, altering, remedying, ameliorating, improving or affecting the disease, the symptoms of disease or disorder or the predisposition toward the disease or disorder, or delaying its onset or progression.

The methods and anti-VEGF-B compounds of the present invention are useful for treating any mammalian subject that has been diagnosed with or is at risk of having a metabolic disorder. A subject in whom the development of a metabolic disorder (e.g., diabetes, obesity, metabolic syndrome, etc.) is being prevented may or may not have received such a diagnosis. One in the art will understand that the subjects may have been subjected to standard tests or may have been identified, without examination, as one at high risk due to the presence of one or more risk factors, which are described below.

Diagnosis of metabolic disorders may be performed using any standard method known in the art, such as those described herein. Methods for diagnosing diabetes are described, for example, in U.S. Pat. No. 6,537,806, incorporated herein by reference. Diabetes may be diagnosed and monitored using, for example, urine tests (urinalysis) that measure glucose and ketone levels (products of the breakdown of fat); tests that measure the levels of glucose in blood; glucose tolerance tests; and assays that detect molecular markers characteristic of a metabolic disorder in a biological sample (e.g., blood, serum, or urine) collected from the mammal (e.g., measurements of Hemoglobin Alc (HbAlc) levels in the case of diabetes).

Patients may be diagnosed as being at risk or as having diabetes if a random plasma glucose test (taken at any time of the day) indicates a value of 200 mg/dL or more, if a fasting plasma glucose test indicates a value of 126 mg/dL or more (after 8 hours), or if an oral glucose tolerance test (OGTT) indicates a plasma glucose value of 200 mg/dL or more in a blood sample taken two hours after a person has consumed a drink containing 75 grams of glucose dissolved in water. The OGTT measures plasma glucose at timed intervals over a 3-hour period. Desirably, the level of plasma glucose in a diabetic patient that has been treated according to the invention ranges between 160 to 60 mg/dL, between 150 to 70 mg/dL, between 140 to 70 mg/dL, between 135 to 80 mg/dL, and preferably between 120 to 80 mg/dL.

Optionally, a hemoglobin Alc (HbAlc) test, which assesses the average blood glucose levels during the previous two and three months, may be employed. A person without diabetes typically has an HbAlc value that ranges between 4% and 6%. For every 1% increase in HbAlc, blood glucose levels increases by approximately 30 mg/dL and the risk of complications increases. Preferably, the HbAlc value of a subject being treated according to the present invention is reduced to less than 9%, less than 7%, less than 6%, and most preferably to around 5%. Thus, the HbAlc levels of the subject being treated are preferably lowered by 10%, 20%, 30%, 40%, 50%, or more relative to such levels prior to treatment.

Gestational diabetes is typically diagnosed based on plasma glucose values measured during the OGTT. Since glucose levels are normally lower during pregnancy, the threshold values for the diagnosis of diabetes in pregnancy are lower than in the same person prior to pregnancy. If a woman has two plasma glucose readings that meet or exceed any of the following numbers, she has gestational diabetes: a fasting plasma glucose level of 95 mg/dL, a 1-hour level of 180 mg/dL, a 2-hour level of 155 mg/dL, or a 3-hour level of 140 mg/dL.

Ketone testing may also be employed to diagnose type I diabetes. Because ketones build up in the blood when there is not enough insulin, they eventually accumulate in the urine. High levels of blood ketones may result in a serious condition called ketoacidosis.

The use of any of the above tests or any other tests known in the art may be used to monitor the efficacy of the present treatment. Since the measurements of hemoglobin Alc (HbAlc) levels is an indication of average blood glucose during the previous two to three months, this test may be used to monitor a patient's response to diabetes treatment.

Abnormalities of fatty-acid metabolism are increasingly recognized as key components of the pathogenesis of the metabolic syndrome and type-II diabetes (McGarry, J. Science, 258:765-770, 1992). Fat-feeding and raised levels of circulating FFAa are clearly sufficient to induce peripheral and hepatic insulin resistance. Accumulation of lipids inside muscle cells (Krssak et al., Diabetologin., 42:113-116, 1999) and specific increases in muscle long-chain fatty-acyl-CoA content (Ruderman et al., Am. J. Physiol., 276:E1-E18, 1999) have been implicated in causing insulin resistance. In additional, lipid accumulation within pancreatic islets has been proposed to impair insulin secretion (Unger, R., Diabetes, 44:863-870, 1993). Thus, it is contemplated that he anti-VEGF-B compounds of the invention can be used to treat or prevent insulin resistance associated with diabetes, obesity and metabolic syndrome or symptoms associated therewith.

One category of subjects to be treated according to the invention are subjects with metabolic syndrome. Metabolic syndrome (also referred to as syndrome X) is a cluster of risk factors that is responsible for increased cardiovascular morbidity and mortality. Metabolic syndrome is typically characterized by a group of metabolic risk factors that include 1) central obesity; 2) atherogenic dyslipidemia (blood fat disorders comprising mainly high triglycerides (“TG”) and low HDL-cholesterol (interchangeably referred to herein as “HDL”) that foster plaque buildups in artery walls); 3) raised blood pressure; 4) insulin resistance or glucose intolerance (the body can't properly use insulin or blood sugar); 5) prothrombotic state (e.g., high fibrinogen or plasminogen activator inhibitor in the blood); and 6) a proinflammatory state (e.g., elevated high-sensitivity C-reactive protein in the blood). The National Cholesterol Education Program (NCEP) Adult Treatment Panel (ATP) III guidelines define metabolic syndrome by the following five clinical parameters: a) a waist circumference greater than 102 cm for men, and greater than 88 cm for women; b) a triglyceride level greater than 150 mg/dl; c) an HDL-cholesterol less than 40 mg/dl for men, and less than 50 mg/dl for women; d) a blood pressure greater than or equal to 130/85 mmHG; and e) a fasting glucose greater than 110 mg/dl.

Another category of subjects to be treated according to the invention are subjects with dyslipidemias. As used herein, dyslipidemia is an abnormal serum, plasma, or blood lipid profile in a subject. An abnormal lipid profile may be characterized by total cholesterol, low density lipoprotein (LDL)-cholesterol, triglyceride, apolipoprotein (apo)-B or Lp(a) levels above the 90^{th} percentile for the general population or high density lipoprotein (HDL)-cholesterol or apo A-1 levels below the 10^{th} percentile for the general population. Dyslipidemia can include hypercholesterolemia and/or hypertriglyceridemia. Hypercholesterolemic human subjects and hypertriglyceridemic human subjects are associated with increased incidence of cardiovascular disorders. A hypercholesterolemic subject has an LDL cholesterol level of >160 mg/dL, or >130 mg/dL and at least two risk factors selected from the group consisting of male gender, family history of premature coronary heart disease, cigarette smoking, hypertension, low HDL (<35 mg/dL), diabetes mellitus, hyperinsulinemia, abdominal obesity, high lipoprotein, and personal history of a cardiovascular event. A hypertriglyceridemic human subject has a triglyceride (TG) level of >200 mg/dL.

Dyslipidemias encompassed by this invention include dyslipidemias caused by single gene defects, dyslipidemias that are multifactorial or polygenic in origin, as well as dyslipidemias that are secondary to other disease states or secondary to pharmacological agents. Examples of genetic dyslipidemias include Familial Hypercholesterolemia, Familial Defective Apo B 100, Familial Hypertriglyceridemia, Familial Apoprotein CII deficiency, Hepatic Lipase Deficiency, Familial Combined Hyperlipidemia, Dysbetalipoproteinemia, and Familial Lipoprotein Lipase Deficiency.

Another category of subjects to be treated according to the invention are subjects with cardiovascular disorders. “Cardiovascular disorder”, as used herein, includes elevated blood pressure, atherosclerosis, heart failure or a cardiovascular event such as acute coronary syndrome, myocardial infarction, myocardial ischemia, chronic stable angina pectoris, unstable angina pectoris, angioplasty, stroke, transient ischemic attack, claudication(s), or vascular occlusion(s).

Risk factors for a cardiovascular disorder include dyslipidemia, obesity, diabetes mellitus, pre-hypertension, elevated level(s) of a marker of systemic inflammation, age, a family history of cardiovascular disorders, and cigarette smoking. The degree of risk of a cardiovascular disorder or a cardiovascular event depends on the multitude and the severity or the magnitude of the risk factors demonstrated by the subject. Risk charts and prediction algorithms are available for assessing the risk of cardiovascular disorders and cardiovascular events in a human subject based on the presence and severity of risk factors.

The anti-VEGF-B compounds of the invention can be administered alone or in combination with one or more further pharmacologically active substances which have, for example, favorable effects on metabolic disturbances or disorders frequently associated therewith. Examples of such medicaments are medicaments which lower blood glucose, anti-diabetics, active ingredients for the treatment of dyslipidemias, anti-atherosclerotic medicaments, anti-obesity agents, anti-inflammatory active ingredients, active ingredients for the treatment of malignant tumors, anti-thrombotic active ingredients, active ingredients for the treatment of high blood pressure, active ingredients for the treatment of heart failure and active ingredients for the treatment and/or prevention of complications caused by diabetes or associated with diabetes.

Furthermore, the anti-VEGF-B compounds may be administered in combination with one or more anti-hypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, alatriopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. Any suitable combination of the compounds according to the invention with one or more of the above-mentioned anti-VEGF-B compounds and optionally one or more further pharmacologically active substances are considered to be within the scope of the present invention.

At present, therapy for diabetes (i.e., type II diabetes) relies mainly on several approaches intended to reduce the hyperglycaemia itself: sulphonylureas (and related insulin secretagogues), which increase insulin release from pancreatic islets; metformin, which acts to reduce hepatic glucose production; peroxisome proliferators-activated receptor-γ (PPAR-γ) agonists (thiazolidinediones), which enhance insulin action; α-glucosidase inhibitors, which interfere with gut glucose absorption; and insulin itself, which suppresses glucose production and augments glucose utilization.

The one or more further pharmacologically active substances can be combined with the anti-VEGF-B compounds of the invention in particular for a synergistic improvement in the effect. Administration of the active ingredient combination can take place either by separate administration of the active ingredients to the patient or in the form of combination products.

The anti-VEGF compounds can also be administered in combination with selective serotonin uptake inhibitors or anti-psychotic drugs, which often induce obesity and diabetes. Treatment with anti-VEGF compounds can also be combined with a regimen of exercise.

Therapeutic Formulations

Therapeutic formulations of the compositions useful for practicing the present invention such as VEGF-B inhibitors may be prepared for storage by mixing the selected composition having the desired degree of purity with optional physiologically pharmaceutically-acceptable carriers, excipients, or stabilizers (Remington's Pharmaceutical Sciences, 18th edition, A. R. Gennaro, ed., Mack Publishing Company (1990)) in the form of a lyophilized cake or an aqueous solution. Acceptable carriers, excipients or stabilizers are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

The composition to be used for in vivo administration should be sterile. This is readily accomplished by filtration through sterile filtration membranes, prior to or following lyophilization and reconstitution. The composition for parenteral administration ordinarily will be stored in lyophilized form or in solution.

Therapeutic compositions generally are placed into a container having a sterile access port, for example, an intravenous solution bag or vial having a stopper pierceable by a hypodermic injection needle. The route of administration of the composition is in accord with known methods, e.g. oral, injection or infusion by intravenous, intraperitoneal, intracerebral, intramuscular, intraocular, intraarterial, or intralesional routes, or by sustained release systems or implantation device. Where desired, the compositions may be administered continuously by infusion, bolus injection or by implantation device.

Suitable examples of sustained-release preparations include semi-permeable polymer matrices in the form of shaped articles, e.g. films, or microcapsules. Sustained release matrices include polyesters, hydrogels, polylactides (U.S. Pat. No. 3,773,919, EP 58,481), copolymers of L-glutamic acid and gamma ethyl-L-glutamate (Sidman, et al., Biopolymers, 22: 547-556 (1983)), poly(2-hydroxyethyl-methacrylate) (Langer, et al., J. Biomed. Mater. Res., 15:167-277 (1981) and Langer, Chem. Tech., 12:98-105 (1982)), ethylene vinyl acetate (Langer, et al., supra) or poly-D(−)-3-hydroxybutyric acid (EP 133,988). Sustained-release compositions also may include liposomes, which can be prepared by any of several methods known in the art (e.g., DE 3,218,121; Epstein, et al., Proc. Natl. Acad. Sci. USA, 82:3688-3692 (1985); Hwang, et al., Proc. Natl. Acad. Sci. USA, 77:4030-4034 (1980); EP 52,322; EP 36,676; EP 88,046; EP 143,949).

An effective amount of the compositions to be employed therapeutically will depend, for example, upon the therapeutic objectives, the route of administration, and the condition of the patient. Accordingly, it will be necessary for the therapist to titer the dosage and modify the route of administration as required to obtain the optimal therapeutic effect. A typical daily dosage may range from about 1 μg/kg to up to 100 mg/kg or more, depending on the factors mentioned above. Typically, a clinician will administer the composition until a dosage is reached that achieves the desired effect. The progress of this therapy is easily monitored by conventional assays designed to evaluate blood glucose levels or other particular conditions of interest in a particular subject.

Pharmaceutical compositions may be produced by admixing a pharmaceutically effective amount of a VEGF-B inhibitor with one or more suitable carriers or adjuvants such as water, mineral oil, polyethylene glycol, starch, talcum, lactose, thickeners, stabilizers, suspending agents, etc. Such compositions may be in the form of solutions, suspensions, tablets, capsules, creams, salves, ointments, or other conventional forms.

VEGF-B inhibitors can be used directly to practice materials and methods of the invention, but in preferred embodiments, the compounds are formulated with pharmaceutically acceptable diluents, adjuvants, excipients, or carriers. The phrase “pharmaceutically or pharmacologically acceptable” refers to molecular entities and compositions that do not produce adverse, allergic, or other untoward reactions when administered to an animal or a human, e.g., orally, topically, transdermally, parenterally, by inhalation spray, vaginally, rectally, or by intracranial injection. (The term parenteral as used herein includes subcutaneous injections, intravenous, intramuscular, intracisternal injection, or infusion techniques. Administration by intravenous, intradermal, intramusclar, intramammary, intraperitoneal, intrathecal, retrobulbar, intrapulmonary injection and/or surgical implantation at a particular site is contemplated as well.) Generally, this will also entail preparing compositions that are essentially free of pyrogens, as well as other impurities that could be harmful to humans or animals. The term “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Routes of Administration

The therapeutic compositions are administered by any route that delivers an effective dosage to the desired site of action, with acceptable (preferably minimal) side-effects. Numerous routes of administration of agents are known, for example, oral, rectal, transmucosal, or intestinal administration; parenteral delivery, including intramuscular, subcutaneous, intramedullary injections, as well as intrathecal, intraperitoneal, intranasal, cutaneous or intradermal injections; inhalation, and topical application. However, localized routes of administration directed to the tissue(s) or organ(s) that are the target of treatment is preferred. This may be the brain, or spine in certain neurodegenerative disorders; the muscles and/or heart in other disorders; the liver or kidneys, the intestine, etc.

Therapeutic dosing is achieved by monitoring therapeutic benefit in terms of any of the parameters outlined herein (speed of wound healing, reduced edema, reduced complications, etc.) and monitoring to avoid side-effects. Preferred dosage provides a maximum localized therapeutic benefit with minimum local or systemic side-effects. Suitable human dosage ranges for the polynucleotides or polypeptides can be extrapolated from these dosages or from similar studies in appropriate animal models. Dosages can then be adjusted as necessary by the clinician to provide maximal therapeutic benefit for human subjects.

Compositions and Formulations

Compositions for use in accordance with the present invention may be formulated in a conventional manner using one or more physiologically acceptable carriers comprising excipients and auxiliaries that facilitate processing of a therapeutic composition into preparations which can be used pharmaceutically. These pharmaceutical compositions may be manufactured in a manner that is itself known, e.g., by means of conventional mixing, dissolving, granulating, dragee-making, levigating, emulsifying, encapsulating, entrapping or lyophilizing processes. Proper formulation is dependent upon the route of administration chosen.

When a therapeutically effective amount of a composition of the present invention is administered by e.g., intradermal, cutaneous or subcutaneous injection, the composition is preferably in the form of a pyrogen-free, parenterally acceptable aqueous solution. The preparation of such parenterally acceptable protein or polynucleotide solutions, having due regard to pH, isotonicity, stability, and the like, is within the skill in the art. A preferred composition should contain, in addition to protein or other active ingredient of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The composition of the present invention may also contain stabilizers, preservatives, buffers, antioxidants, or other additives known to those of skill in the art. The agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compositions can be formulated readily by combining the active compounds with pharmaceutically acceptable carriers well known in the art. Such carriers enable the compounds of the invention to be formulated as tablets, pills, dragees, powders, capsules, liquids, solutions, gels, syrups, slurries, suspensions and the like, for oral ingestion by a patient to be treated.

For administration by inhalation, the compositions for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas.

Pharmaceutical formulations for parenteral administration include aqueous solutions of the compositions in water-soluble form. Optionally, the suspension may also contain suitable stabilizers or agents which increase the solubility of the compositions to allow for the preparation of highly concentrated solutions. Alternatively, the active ingredient may be in powder form for constitution with a suitable vehicle, e.g., sterile pyrogen-free water, before use.

The compositions may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

The compositions also may comprise suitable solid or gel phase carriers or excipients.

The compositions of the invention may be in the form of a complex of the protein(s) or other active ingredient of present invention along with protein or peptide antigens.

The compositions may include a matrix capable of delivering the protein-containing or other active ingredient-containing composition to the site of tissue damage, providing a structure for the developing bone and cartilage and optimally capable of being resorbed into the body. Such matrices may be formed of materials presently in use for other implanted medical applications. The choice of matrix material is based on biocompatibility, biodegradability, mechanical properties, cosmetic appearance and interface properties.

The composition may further contain other agents which either enhance the activity of the protein or other active ingredient or complement its activity or use in treatment. Such additional factors and/or agents may be included in the pharmaceutical composition to produce a synergistic effect with protein or other active ingredient of the invention, or to minimize side effects.

Techniques for formulation and administration of the therapeutic compositions of the instant application may be found in “Remington's Pharmaceutical Sciences,” Mack Publishing Co., Easton, Pa., latest edition. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

All references cited herein are incorporated herein in their entirety.

The following examples serve to further illustrate the present invention.

### EXAMPLE 1

### Materials and Methods

Bioinformatics. Microarray expression data from 44 datasets, all generated using the Affymetrix U74A platform, were downloaded from the GEO database (Barrett et al. (2007) Nucleic Acids Res 35, D760-765). Arrays were intensity normalized and merged into one 708 array dataset. A hierarchical clustering procedure (average group linkage, Pearson correlation, threshold r>0.8) was applied to identify groups of coexpressed genes.

The GNF SymAtlas database (Su et al. (2006) Proc Natl Acad Sci USA 99, 4465-4470) was used to find mouse transcripts correlating (cut-off value ≧0.80) with the expression of mouse Vegfb, Pgf (termed Plgf in the manuscript for clarity) or Vegfa transcripts. In order to classify the correlated transcripts according to their subcellular localization, GeneOntology Component information from NCBI Entrez Gene was used.

Animals. C57BL/6N-VEGF-B^{-/-} mice have previously been described by Aase et al. (2001) Circulation 104:358-364. The mice were housed in a 12 hrs light/dark cycle on normal mouse chow. All animal work was conducted in accordance to the Swedish Animal Research Committee at Karolinska Institutet, Stockholm, Sweden.

Quantitative RT-PCR (qPCR) analysis. Tissues from 8-12 week male wt and VEGF-B^{-/-} mice were dissected, frozen in liquid nitrogen and kept at −80° C. Oxidative muscle (M. soleus) was used in all studies, apart from where glycolytic muscle (the quadriceps femoris muscle group) was also analyzed. RNA was isolated with TRIzol reagent (Invitrogen) and QIAGEN RNeasy (Qiagen) according to the manufacturers' instructions. For expression studies in cell cultures (see below), MS-1 and bEnd3 cells were washed with PBS, lysed with RLT buffer (Qiagen) and passed trough a shredder column (Qiagen) and RNA was isolated using RNAeasy (Qiagen).

Total RNA (1 μg) was reverse transcribed according to the manufacturer's instructions (iScript cDNA synthesis kit, Bio-Rad). qPCR was performed using Platinum SYBR green SuperMix (Invitrogen) and 25 ng cDNA per reaction. Primer sequences are listed in Table 3. Expression levels were normalized to the expression of L19 and/or β₂-microglobulin.

Mitochondrial DNA copy number determination. Total DNA from hearts of male wt and VEGF-B^{-/-} mice was phenol-chloroform extracted. The final aqueous phase was used to assess mtDNA content relative to nDNA content by qPCR, using 2 ng of total DNA as template and primers for cytochrome c oxidase II (Cox II, mitochondrial genome) and Gapdh (nuclear genome). Primer sequences are listed in Table 3.

Fatty acid uptake in vivo. 8-12 week male wt and VEGF-B^{-/-} mice were given a bolus dose of 2 μCi (36.6 nmol) of 1-¹⁴C—OA (PerkinElmer) dissolved in olive oil by oral gavage. Blood samples were drawn from the tail vein at 15, 30, 60 and 120 min post-gavage, plasma was extracted by centrifugation for 1 min at 16,000 g, and solubilized 1:6 in NCS-II Tissue Solubilizer (Amersham). BCS-NA Counting Scintillant (Amersham) was added and total radioactivity was measured by liquid scintillation. For analysis of lipid uptake into organs, the mice were anesthetized with Avertin 2 or 24 hrs post-gavage, perfused with PBS, and organs were dissected. The organs were dissolved o/n at 50° C. in Tissue Solubilizer (1 ml/100 mg tissue), neutralized with 30 □l/ml glacial acetic acid and BCS-NA Counting Scintillant was added. Total radioactivity was measured by liquid scintillation using a Tri-Carb 1600 TR Liquid Scintillator (Packard).

Plasma analysis. Glucose was measured from the tail vein of normally fed or over-night fasted age-matched 8-12 week old male mice using a Contour Blood Glucose Meter (Ascensia). For all the other analysis, blood was collected from hearts of Avertin-anesthetized mice using EDTA syringes, and centrifuged for 1 min at 16,000 g. TG and NEFA content in plasma was determined using the Serum Triglyceride Determination Kit (Sigma), and the Wako NEFA C test kit (Wako Diagnostics), respectively, according to the manufactures' instructions. Plasma β-hydroxybutyrate was analyzed using the β-hydroxybutyrate LiquiColor Assay from Stanbio Laboratory (Boerne, Tex., USA). Plasma leptin was assayed using the Mouse Leptin Quantikine Immunoassay (R&D Systems).

Oil red O staining. Heart, oxidative (M. soleus) muscle and liver from age and sex matched wt and VEGF-B^{-/-} mice were dissected, rinsed in PBS and imbedded in Tissue-Tek (Sakura). Cryosections (12 μm) were immersed 5-15 min in oil red O working solution (2.5 g oil red O (Sigma-Aldrich), dissolved in 400 ml 99% isopropanol, further diluted 6:10 in H₂O, and finally filtered through a 0.45 μm filter (Corning). Sections were rinsed in H₂O, counterstained with Mayer's Hematoxylin (Histolab), immersed in a 1:5 dilution of saturated Li₂CO₃ and rinsed 10 min under running tap water before they were mounted. Stained sections were examined by light field microscopy and >5 randomly selected fields were photographed in each section. Lipid droplets in heart were quantified by counting the number of stained droplets in each frame. For quantification of droplets in muscle and liver the amount of red pixels in each frame was calculated using images imported into Adobe Photoshop. All experiments were repeated three times.

Indirect calorimetry, MRI and fat pad weight. MRI and indirect calorimetry of 16-18 week old male wt and VEGF-B^{-/-} mice was performed at the Centre for mouse physiology and imaging core facility at Göteborg University, Sweden, essentially as previously described. (Faldt et al (2004) Endocrinology 145, 2680-2686; Garcia et al (2006) Diabetes 55, 1205-1213). Indirect calorimetry measuring VO₂ and VCO₂ was performed at 21° C. for 23 hrs using a Somedic INCA system (Hörby, Sweden). Experiment time was between 11 am to 10 am (lights on: 11:00-19:00 and 07:00-10:00, lights off: 19:00-07:00). During the measurements, the animal had free access normal chow and tap water. Two animals from each group were analyzed in parallel. The statistics was performed on data obtained during three periods (Afternoon; 13:00-19:00, Night; 19:00-07:00 and Morning; 07:00-10:00). The first two experiment hours (11:00-13:00) were excluded from the analysis. For oxygen consumption and carbon dioxide output, data is shown as normalized to body weight^{̂0.75} (VO₂^{0.75} and VCO₂O^{0.75}), the unit for both measurements being ml/mg/kg. Respiratory Quotient (RQ) was calculated as the ratio of VCO₂ to VO₂. Mean metabolic rate (expressed as kcal/h/kĝ0.75) during the 23 hrs was calculated using Weir's equation (Weir (1949) J Physiol 109, 1-9).

For MR imaging, mice were anesthetized and placed in the centre of a 7 Tesla MR system (Burker BioSpin). 18 axial and coronal spin-echo measurements per mouse (1 mm slice thickness with 3.5 mm slice distance) were obtained covering all regions of the body, and the measurements were subsequently repeated with fat suppression to eliminate fast-flow regions. MR images were post-processed using the ImageJ software, and body fat percentage was calculated from the axial images (n=18/mouse) as previously described by Garcia et al. (2006) Diabetes 55, 1205-1213. Fat suppressed images were used to eliminate false positive white signals. The percentage was calculated as total area of true fat, normalized to total mouse area. To confirm the MRI results regarding fat mass, 16-18 week old male mice were killed and four fat pads (eWAT, rWAT (both intra-abdominal), iWAT and scWAT (both subcutaneous)) were dissected and weighed.

Voluntary activity measurements. Activity measurements were done as described by Aase et al. (2001) Circulation 104, 358-364 and Johansson et al. (1997) Acta Physiol Scand 160, 133-138

Lipolysis assay. Glycerol release from cells was monitored as a measure of triglyceride lysis and performed as described by Polak et al. (2008) Cell Metab 8, 399-410. Freshly isolated eWAT pieces from wt and VEGF-B^{-/-} mice were washed in PBS, weighted and incubated for 14 hrs in DMEM. The WAT was removed and glycerol content of the medium was measured as described for plasma TGs.

β-oxidation. The ex vivo β-oxidation assay of both C2C12 cells and tissue lysates was adopted from previous publications (Fruebis et al. (2001) Proc Natl Acad Sci USA 98, 2005-2010; Mao et al. (2006) Nat Cell Biol 8, 516-523) and measures the total amount of carbon dioxide released from completely oxidizing the first carbon of the fatty acid used, 1-C¹⁴-oleic acid, irrespective of if this is preformed by the cell mitochondria or peroxisomes. Minor modifications were made for tissue β-oxidation whereas the C2C12 assay was carried out exactly as described by Mao et al. (2006) Nat Cell Biol 8, 516-523. Globular adiponectin (R&D Systems) was used as a positive control as described in the reference. For measuring tissue oxidation, heart, oxidative muscle, iBAT, liver and eWAT from PBS-perfused 18 week old male wild-type and VEGF-B^{-/-} mice were dissected, weighted and minced, and there after pre-incubated at 37° C. in a 5% CO₂ atmosphere for 1.5 hrs in 24 well plates together with 500 μl β-oxidation buffer (DMEM (Gibco), containing 12 mM glucose, 4 mM glutamine, 25 mM Hepes, 1% fatty acid free BSA and 0.25 mM oleate). For the oxidation reaction, 0.5 μCu (1 μCu/ml final concentration or 9.15 nmol) 1-¹⁴C-oleic acid was added to the wells. The wells were then covered with 2×2 cm membranes (Whatman filter paper #3) and incubated an additional 1.5 hrs in 37° C. Formed ¹⁴CO₂ was collected 1 h in RT onto the membranes by first alkalinizing them with 3M NaOH, and then adding 500 μl 70% perchloric acid to the wells underneath. Membranes were dried o/n, and radioactivity of the membranes was assessed by liquid scintillation using a water-soluble scintillation liquid, Emulsifier Safe Liquid Scintillation Coctail (PerkinElmer).

PET scan. Positron emission tomography (PET) was performed on a Focus120 MicroPET® (CTI Concorde Microsystems). The PET data were processed with MicroPET manager and evaluated using the ASIPro VM software (CTI Concorde Microsystems). 8-12 week old non-starved male wt and VEGF-B^{-/-} mice were anaesthetized with isoflurane (5% initially and 1.5% to maintain anesthesia) and placed on the camera bed on a heating pad (37° C.). Under these experimental conditions all [¹⁸F]FDG will be taken up by heart, or excreted to the bladder. [¹⁸F]FDG was obtained from batches used for clinical PET imaging, which had passed radiochemical and pharmacological analysis. Mice received tail vein i.v. injections of 2.8-7.6 MBq [¹⁸F]FDG in a volume of 150 to 200 μl. Two animals from each group were analyzed in parallel. The 60 min PET data acquisition started at injection. At the end of the scan mice were euthanized and the hearts were dissected and measured in a dose calibrator (Capintec). All data were normalized by the specific dose at the time of injection for each mouse.

Immunoblotting. Heart and liver from 8-12 week old male wt and VEGF-B^{-/-} mice were frozen in liquid nitrogen and kept at −80° C. Frozen tissues were homogenized in tissue lysis buffer (50 mM Tris-HCl pH 8.0, 150 mM NaCl₂, 1% Triton-X100, 0.5% deoxycholic acid and 0.1% SDS) supplemented with Complete Protease Inhibitor Cocktail (Roche). Tissue lysates were incubated on ice for 30 min followed by centrifugation at 16,000 g at 4° C. for 20 min where after the lysates were subjected to SDS-PAGE on NuPage 12% Bis-Tris gels (Invitrogen) under reducing conditions. Separated proteins were blotted onto filters using the iBlot system (Invitrogen). FATP3 was detected with a rabbit antiserum raised against a peptide corresponding to amino acid residues 24-42 of mFATP3, whereas an affinity purified polyclonal rabbit anti-mFATP4 antibody (Santa Cruz), and goat anti-mCD36 antibody (R&D systems) were used to detect FATP4 and CD36. Blotting with goat polyclonal anti-mouse calnexin antibody (Santa Cruz) was used as loading control. Bound antibodies were detected with Enhanced Chemillumisence Plus reagent (Amersham).

For protein analysis of MS-1 and bEnd3 cells (see below), cells were washed in PBS and incubated in cell lysis buffer (20 mM Tris-HCl pH 7.5, 150 mM NaCl, 5 mM EDTA, 0.5% deoxycholic acid and 0.5% Triton-X100) on ice for 10 min. Cells were detached by scraping, and centrifuged for 10 min at 10,000 g at 4° C. For immunoprecipitation, 2 μg of affinity purified α-mFATP4 antibody (Santa Cruz) was added to the suspension and incubated end-over-end for 90 min at 4° C. Protein-A beads were added, and the mixture was further incubated for 30 min. Precipitated proteins were subjected to SDS-PAGE and immunoblotting as described above.

Isolation of endothelial cells (ECs) from mouse heart. Male age-matched wild-type and VEGF-B^{-/-} mice were anesthetized with Avertin and perfused with cold PBS. Hearts were dissected, diced and incubated for 50 min at 37° C. under shaking with EC-buffer (HBSS (Invitrogen), 1% BSA, 25 mM Hepes and 5% Cell Dissociation buffer (GIBCO)) supplemented with 5 mg/ml Collagenase A (Roche Diagnostics) and 100 U/ml DNase I (Invitrogen). The cell suspension was filtered 2× through a 70 μm nylon mesh (Falcon) followed by 2× filtrations trough a 40 μm nylon mesh. The meshes were washed with ice-cold EC-buffer supplemented with 100 U/ml DNase I, and the suspension was centrifuged at 200 g for 10 min at 4° C. The cell pellets were resuspended in 1 ml EC-buffer and incubated for 30 min at 4° C. with 50 μl Dynabeads Biotin Binder (Dynal), which had been pre-coated according to the manufacturer's instructions with 3 mg biotinylated anti-CD31 antibody (BD Biosciences, Pharingen). ECs bound to the beads were isolated using a magnetic particle collector, and the remaining cell suspension (non-EC fraction) was collected, centrifuged for 2 min at 200 g, and the cell pellets were saved. EC-beads were washed 10 times with EC-buffer and both EC and non-EC cells were lysed in RLT buffer (Qiagen) and passed trough a Shredder column (Qiagen). RNA was isolated and reverse transcribed as described above, using 100-250 ng RNA per reaction. The purity of the isolated cell fractions (EC and non-EC) were analyzed by conventional RT-PCR of marker genes (primer sequences in shown in Table 3) using 5 ng of EC, or non-EC, cDNA per reaction. Tissue cDNA from heart in a 3-5 times excess was used as positive controls. The relative expression levels of the FATPs in EC and non-EC fractions were determined by qPCR as described above.

HE staining and Immunohistochemistry. Heart, muscle, iBAT and eWAT from male age-matched wt and VEGF-B^{-/-} mice were PFA-perfused, dissected and post-fixated in 4% PFA o/n, and subsequently processed for paraffin imbedding using standard procedures. iBAT and eWAT sections were HE stained, mounted with Sakura mounting medium before analysis by bright field microscopy. For quantification of the amount of lipid vacuoles in HE stained iBAT sections, the number of pink pixels from the eosin staining in each frame was calculated using images imported into Adobe Photoshop, and subtracted from the total number of pixels in each frame, giving the amount of non-stained pixels. These quantifications were done on 3 fields/section from 3 independent experiments (n=7 mice per genotype).

Immunolocalization of FATP3, FATP4, and slow-twitch myosin in heart and muscle was performed using Elite ABC Vectastain and Vector M.O.M. Immunodetection kit (Vector laboratories) using a mouse anti-FATP3 mAb (Abcam), an affinity-purified polyclonal rabbit anti-FATP4 antibody (Santa Cruz), and a mouse mAb against type I (slow) skeletal myosin (GeneTex Inc.). Antigen retrieval was obtained by heating tissue slides in 0.01 M citrate buffer, pH 6.0, at 95° C. for 20 min followed by treatment with 0.25 mg/ml trypsin for 30 min at 37° C. Negative controls were stained simultaneously using only the secondary antibodies. Sections were counterstained with hematoxylin and mounted with Sakura mounting medium before analysis by bright field microscopy.

Adenoviral transductions. 10-13 week old male C57Bl/6J mice were used. The mice were housed in standard housing conditions in The National Laboratory Animal Center of Kuopio University, and all animal procedures were carried out in accordance with the guidelines of the Experimental Animal Committee of Kuopio University. Adenoviral construct encoding hVEGF-B₁₈₆, hVEGF-A₁₆₅, mPlGF2 or LacZ were used in this study. The human clinical grade first generation serotype 5 replication deficient (E1, partially E3 deleted) adenovirus stocks were produced in 293 cells as described by Rissanen et al. (2003) Circ Res 92, 1098. Adenoviral constructs (6 to 17 μl of 1×10¹²viral particles/ml) were injected to the anterior wall of the left ventricle by ultrasound guidance. The injections were carried out as described by Springer et al. (2005) Am J Physiol Heart Circ Physiol 289, H1307-1314. Analgesic (carprofen 50 mg/ml, Rimadyl) was given after the injections. The mice were sacrificed 6 days post injection by cervical dislocation. The hearts were perfused with PBS, the left ventricle was dissected and frozen in liquid nitrogen. RNA expression was analyzed as described above.

Cell culture. bEnd3 cells (ATCC) were cultured in high glucose DMEM (Gibco) supplemented with 10% FCS, 3.7 g/l NaHCO₃, 2 mM glutamine, 100 U/ml penicillin and 100 μg/ml streptomycin according to the manufacturers instructions. Mile Sven-1 cells (MS-1, kind gift from Dr. Theresa Vincent) were grown on 1% gelatin coated plates in Endothelial Cell basal Medium MV2 (Promo Cell) supplemented with MV2 Supplement pack growth factors (Promo Cell), 100 U/ml penicillin and 100 μg/ml streptomycin according to the manufacturers instructions. mVEGF-B₁₈₆, mVEGF-B₁₆₇, mPlGF2, mVEGF-A₁₆₄, soluble hVEGFR1 and neutralizing antibodies against mVEGFR1, mVEGFR2 and rat NRP-1 were purchased from R&D systems. The anti-VEGF-B mAb 2H10 (Scotney et al. (2002) Clin Exp Pharmacol Physiol 29, 1024-1029), was a kind gift from Dr. Andrew Nash, Commonwealth Serum Laboratories (Melbourne, Australia). For treatment of MS-1 and bEnd3 cells with VEGF-B, PlGF2, VEGF-A or mock treatment, cells were starved in growth medium containing 0.5% FCS for 4-12 hrs followed by addition of 300 ng/ml of the growth factors, or 1 μg/ml of sVEGFR1 or anti-VEGF-B mAb, or only new medium for mock treatment, and further incubated for 20 hrs. In another set of experiments, cells were pre-incubated 2 hrs with neutralizing antibodies against VEGFR1 (4 μg/ml), VEGFR2 (0.3 μg/ml), or NRP-1 (1 μg/ml), and VEGF-B₁₈₆ was then added to the medium as described above. As a control, cells were also treated with antibodies alone, which showed not to have an effect on FATP expression (data not shown). For competition experiments 1 μg/ml of PlGF-2 was added together with 100 ng/ml VEGF-B₁₈₆. Total RNA fractions or protein lysates (see above) were generated as described before. All experiments were performed in triplicates, and were repeated at least three times.

Cloning of hFATP3 and mFATP4 plasmids. The hFATP3-pOTB7 plasmid containing the entire, sequenced hFATP3 mRNA was purchased from GeneService. The EST clone mFATP4-pYX-Asc (IMAGp998O2014352Q1) was purchased from Rzpd, Germany. Both cDNAs were cut out (EcoRI/XhoI for hFATP3; EcoRI/NotI for mFatp4), the inserts were gel-purified using DNA gel Extraction Kit (Genomics), and directly ligated into the expression vector pcDNA3.1 (Invitrogen). The plasmids were verified by sequencing.

BODIPY-uptake assay. bEnd3 cells were seeded in 24 well plates and cultured as described above using growth medium containing 1% fatty acid free BSA (FF-BSA) and no FCS for the experiments. Cells were transfected with hFATP3-pcDNA3.1, mFATP4-pcDNA3.1, with both plasmids simultaneously, or with empty vector (mock) using the Lipofectamin LTX transfection system together with Plus-reagent (both from Invitrogen) according to the manufacturer's instructions.

In another set of experiments, to explore the VEGF-B mediated effect on lipid uptake, cells were treated with VEGF-B₁₈₆ (300 ng/ml) or anti-VEGF-B mAb (2 μg/ml) for 27 hrs in FF-BSA growth medium. 27 hrs post-transfection, or after growth factor treatment, cells were washed with PBS-1% FF-BSA, and incubated 3 min in 37° C. with PBS-1% FF-BSA-20 μM C₁-BODIPY-C₁₂ (#3823, Sigma). Cells were washed vigorously, fixed with 4% PFA 10 min in RT, and analyzed by inverted microscopy. For each experiment, 3 or more wells were photographed diagonally using a 20× objective and equal exposure times, and all frames (n>10) were used for computer-assisted quantitation of green fluorescent pixels using Photoshop. The experiments were repeated at least 3 times in triplicates.

Targeting FATP expression with siRNAs. Dharmacon specific targeting Accell siRNA towards mFatp3 and mFatp4, as wells as a Red-fluorescence control mouse siRNA kit (including red fluorescent, and non-labeled non-targeting siRNA, and mGapdh targeting siRNA, Dharmacon) was used for this assay. bEnd3 cells were seeded in 24 well plates and transfected with 1 nmol siRNA/well according to the manufacturer's instructions. 48 hrs after transfection the transfection medium was exchanged for growth medium containing 1% FF-BSA and either VEGF-B₁₈₆ or anti-VEGF-B mAb as described above, and the cells were additionally incubated for 24 hrs. Cells were assayed for BODIPY-FA uptake as described above, but they were not fixed. After microscopic analysis the cells were trypsinized, lysed in RLT lysis buffer (Qiagen), and RNA was extracted as described above. The experiment was repeated twice with a total of 6-10 wells analyzed per siRNA transfection.

In vitro lipid transport assay. bEnd3 cells were seeded with a density of 60,000 cells/well on 24-well cell culture inserts (0.4 μm pore size, Falcon). Cells were grown as described above. After treatment of the cells with VEGF-B₁₈₆, PlGF2, VEGF-A, anti-VEGF-B mAb, anti-NRP1 Ab or sVEGFR1 for 30 hrs, 0.5 μCi/well of ¹⁴C-inulin (Amersham) or ¹C-oleic acid (PerkinElmer) was added to the apical chamber, and the cells were further incubated for 4 hrs. To assess the non-saturable background transport, some parallel experiments were performed in the presence of a 10× molar excess (70 μg/ml) sodium oleate (Sigma) together with the ¹⁴C-oleic acid as described by Rohrer et al. (2006) Biochim Biophys Acta 1761, 186-194. For kinetic studies 50 μl samples were collected from the lower compartment every 20 min during the 4 hrs ¹⁴C-oleic acid incubation period, and replaced with 50 μl medium. After 4 hrs, the whole basal liquid compartment was collected, suspended in Emulsifier Safe Liquid Scintillation Cocktail (PerkinElmer), and total radioactivity was measured by liquid scintillation. All experiments were performed in quadruplicates, and repeated at least three times.

Statistics. In all figures data is presented as mean±SEM from pooled data of 2-5 independent experiments. P-values were calculated with one-way ANOVA and two-tailed Students t-test, and p<0.05 was considered significant.

TABLE 1
List of genes included in the mitochondrial/respiration co-expression cluster.

| | |
|---|---|
| 3110001M13Rik | RIKEN cDNA 3110001M13 gene |
| Atp5c1 | ATP synthase, H+ transporting, mitochondrial F1 complex, gamma polypeptide 1 |
| Atp5c1 | ATP synthase, H+ transporting, mitochondrial F1 complex, gamma polypeptide 1 |
| Ndufa8 | NADH dehydrogenase (ubiquinone) 1 alpha subcomplex, 8 |
| Ndufs1 | NADH dehydrogenase (ubiquinone) Fe—S protein 1 |
| Sdhc | succinate dehydrogenase complex, subunit C, integral membrane protein |
| Ndufab1 | NADH dehydrogenase (ubiquinone) 1, alpha/beta subcomplex, 1 |
| — | Mus musculus cDNA clone IMAGE: 5694421, partial cds |
| Atp5o | ATP synthase, H+ transporting, mitochondrial F1 complex, O subunit |
| Ndufb10 | NADH dehydrogenase (ubiquinone) 1 beta subcomplex, 10 |
| 1500032D16Rik | RIKEN cDNA 1500032D16 gene |
| Ndufs3 | NADH dehydrogenase (ubiquinone) Fe—S protein 3 |
| Ogdh | oxoglutarate dehydrogenase (lipoamide) |
| Ndufa9 | NADH dehydrogenase (ubiquinone) 1 alpha subcomplex, 9 |
| Sdhb | succinate dehydrogenase complex, subunit B, iron sulfur (Ip) |
| Ndufa3 | NADH dehydrogenase (ubiquinone) 1 alpha subcomplex, 3 |
| 0710008D09Rik | RIKEN cDNA 0710008D09 gene |
| Ndufc1 | NADH dehydrogenase (ubiquinone) 1, subcomplex unknown, 1 |
| Ndufv1 | NADH dehydrogenase (ubiquinone) flavoprotein 1 |
| Ndufs2 | NADH dehydrogenase (ubiquinone) Fe—S protein 2 |
| Uqcrc1 | ubiquinol-cytochrome c reductase core protein 1 |
| 2310005O14Rik | RIKEN cDNA 2310005O14 gene |
| Ndufb7 | NADH dehydrogenase (ubiquinone) 1 beta subcomplex, 7 |
| Ndufb2 | NADH dehydrogenase (ubiquinone) 1 beta subcomplex, 2 |
| Ndufb9 | NADH dehydrogenase (ubiquinone) 1 beta subcomplex, 9 |
| Np15 | nuclear protein 15.6 |
| Ndufb2 | NADH dehydrogenase (ubiquinone) 1 beta subcomplex, 2 |
| Atp5e | ATP synthase, H+ transporting, mitochondrial F1 complex, epsilon subunit |
| — | Mus musculus cDNA clone IMAGE: 6772417, with apparent retained intron |
| Atp51 | ATP synthase, H+ transporting, mitochondrial F0 complex, subunit g |
| 2610041P16Rik | RIKEN cDNA 2610041P16 gene |
| Cox5b | cytochrome c oxidase, subunit Vb |
| Ndufb8 | NADH dehydrogenase (ubiquinone) 1 beta subcomplex 8 |
| Ndufv2 | NADH dehydrogenase (ubiquinone) flavoprotein 2 |
| Ndufs5 | NADH dehydrogenase (ubiquinone) Fe—S protein 5 |
| Fh1 | fumarate hydratase 1 |
| 2010107E04Rik | RIKEN cDNA 2010107E04 gene |
| Ech1 | enoyl coenzyme A hydratase 1, peroxisomal |
| Etfb | electron transferring flavoprotein, beta polypeptide |
| Dci | dodecenoyl-Coenzyme A delta isomerase (3,2 trans-enoyl-Coenyme A isomerase) |
| Hadhb | hydroxyacyl-Coenzyme A dehydrogenase/3-ketoacyl-Coenzyme A thiolase/enoyl- |
| | Coenzyme A hydratase (trifunctional protein), beta subunit |
| 2610207I16Rik | RIKEN cDNA 2610207I16 gene |
| 2610207I16Rik | RIKEN cDNA 2610207I16 gene |
| Etfdh | electron transferring flavoprotein, dehydrogenase |
| Cpt2 | carnitine palmitoyltransferase 2 |
| Pdha1 | pyruvate dehydrogenase E1 alpha 1 |
| 1110018B13Rik | RIKEN cDNA 1110018B13 gene |
| Cox7a1 | cytochrome c oxidase, subunit VIIa 1 |
| Idh3g | isocitrate dehydrogenase 3 (NAD+), gamma |
| Cs | citrate synthase |
| 1620401E04Rik | RIKEN cDNA 1620401E04 gene |
| AI837181 | expressed sequence AI837181 |
| Vegfb | vascular endothelial growth factor B |
| 0610041L09Rik | RIKEN cDNA 0610041L09 gene |
| Uqcrb | ubiquinol-cytochrome c reductase binding protein |
| 1110007A04Rik | RIKEN cDNA 1110007A04 gene |
| Mor1 | malate dehydrogenase, mitochondrial |

The clustering of co-expressed genes (r > 0.80) was performed as described in Experimental Procedures. Vegfb is emphasized in bold.

TABLE 2
Analysis of mRNA expression of metabolic genes in wt and VEGF-B^{-/-} organs.

| | Organ | | | | |
|---|---|---|---|---|---|
| Gene | Heart | Muscle | iBAT | Liver | eWAT |
| Cpt1a/b | 0.57 ± 0.07 * | 0.61 ± 0.15 * | 0.72 ± 0.04 * | 0.67 ± 0.08 * | 3.60 ± 0.13 *** |
| Cpt2 | 0.76 ± 0.06 ns | 0.75 ± 0.05 * | 0.75 ± 0.11 ns | 0.62 ± 0.10 * | 2.40 ± 0.15 * |
| Slc25a20 | 0.75 ± 0.13 ns | 0.71 ± 0.12 * | 0.63 ± 0.07 ** | 0.60 ± 0.15 * | 1.88 ± 0.12 * |
| Pgc1a | 0.98 ± 0.12 ns | 0.92 ± 0.07 ns | 1.08 ± 0.1 ns | 0.48 ± 0.08 ** | 2.46 ± 0.16 *** |
| Ppara | 1.13 ± 0.08 ns | 0.81 ± 0.08 ns | — | 0.62 ± 0.08 * | 2.83 ± 0.11 *** |
| Pparg | — | — | 0.90 ± 0.12 ns | 0.45 ± 0.08 * | 1.88 ± 0.28 * |
| Acsl | 0.98 ± 0.12 ns | 0.95 ± 0.15 ns | 0.85 ± 0.07 ns | 0.80 ± 0.12 ns | 2.23 ± 18 ** |
| Mcad (Acadm) | 1.01 ± 0.08 ns | 0.93 ± 0.02 ns | 1.14 ± 0.12 ns | 0.52 ± 0.05 ** | 1.50 ± 0.11 * |
| Acox1 | 0.86 ± 0.11 ns | 0.78 ± 0.09 ns | 1.00 ± 0.11 ns | 0.59 ± 0.08 * | 1.15 ± 0.05 ns |
| Ucp1 | — | — | 0.98 ± 0.16 ns | — | 1.43 ± 0.07 * |
| Ucp3 | — | — | 1.34 ± 0.37 ns | — | 2.10 ± 0.1 ** |
| Srebp1c | 1.27 ± 0.06 ns | 1.02 ± 0.08 ns | 2.13 ± 0.20 ** | 0.37 ± 0.25 *** | 0.55 ± 0.01 *** |
| Srebp2 | 1.44 ± 0.03 * | 1.21 ± 0.05 * | 1.28 ± 0.09 * | 0.53 ± 0.16 * | 0.69 ± 0.10 * |
| Acc | 1.58 ± 0.09 * | 1.65 ± 0.12 * | 1.68 ± 0.13 ** | 0.73 ± 0.17 ns | 0.64 ± 0.08 * |
| Glut4 | 1.79 ± 0.12 ** | 1.12 ± 0.07 ns | 1.42 ± 0.07 * | — | — |
| Lpl | 0.63 ± 0.11 * | 0.91 ± 0.09 ns | 1.27 ± 0.10 ns | 0.84 ± 0.12 ns | 1.59 ± 0.12 * |
| Hsl | — | — | — | — | 1.26 ± 0.17 ns |

mRNA levels of genes in lipid metabolism determined by qPCR in tissues from VEGF-B^{-/-} mice, normalized by the mean value of wt mice set to 1 unit (n = 3 mice).
* P < 0.05,
** P < 0.01,
*** P < 0.001.
ns = non significant. Cpt1/2, carnitine palmitoyltransferase, Slc25a20, solute carrier family 25 (mitochondrial carnitine/acylcarnitine translocase), member 20; Pgc1a, PPAR gamma coactivator 1□; Acsl, acyl-CoA synthetase long-chain; Acadm, acyl-Coenzyme A dehydrogenase medium chain; Acox1, acyl-Coenzyme A oxidase; Ucp1/3, Uncoupling protein; Srebp, sterol regulatory element binding transcription factor; Acc, acetyl-CoA carboxylase; Glut4, facilitated glucose transporter 4.

TABLE 3
Primer sequences. All sequences are written 5′ → 3′

| | Fwd primer | Rev primer |
|---|---|---|
| | (SEQ ID NOs 10-56) | (SEQ ID NOs 57-103) |
| mVegfb | TCTGAGCATGGAACTCATGG | TCTGCATTCACATTGGCTGT |
| mNdufa5 | ATCACCTTCGAGAAGCTGGA | ACTTCACCACCCTGAAGCAA |
| mCycs | CCAAATCTCCACGGTCTGTT | CCAGGTGATGCCTTTGTTCT |
| mPlgf | CCCACACCCAGCTCACGTATTTA | TCCCCTCTACATGCCTTCAATGC |
| mVegfa | CAGGCTGCTGTAACGATGAA | TATGTGCTGGCTTTGGTGAT |
| mL19 | GGTGACCTGGATGAGAAGGA | TTCAGCTTGTGGATGTGCTC |
| mBeta actin | ACTCTTCCAGCCTTCCTTC | ATCTCCTTCTGCATCCTGTC |
| mB2m | CTGACCGGCCTGTATGCTAT | CCGTTCTTCAGCATTTGGAT |
| mCpt1a | CATGTCAAGCCAGAGGAAGA | TGGTAGGAGAGCAGCACCTT |
| mCpt1b | CCCATGTGCTCCTACCAGAT | CCTTGAAGAAGCGACCTTTG |
| mCpt2 | CAGCATATGATGGCTGAGTG | GTGGTTTATCCGCTGGTATG |
| mSlc25a20 | TTTGCAGGGATCTTCAACTG | CCCTTTGTACAAGGAGGTGA |
| mPgc1a | GGAGCCGTGACCACTGACA | TGGTTTGCTGCATGGTTCTG |
| mPpara | GACAAGGCCTCAGGGTACCA | GCCGAATAGTTCGCCGAAA |
| mPparg | CCATTCTGGCCCACCAAC | AATGCGAGTGGTCTTCCATCA |
| mAcsl | ATATCTACCTGCGGAGTGAAG | CCTTCCCAAGTTTCAACAAGTC |
| mAcadm | TCGGAGGCTATGGATTCAAC | CAGCCTCTGAATTTGTGCAG |
| mAcox1 | ACTTGTTTGAGTGGGCCAAG | AGAGATTCGGCCTCTCTGTG |
| mHsd17b4 | CGGTTTTGAGAAGCCCATATT | ATTCTGTTTCCTTCCTTCCACA |
| mHmgcs1 | CAGTACTCACCTCAGCAGTTG | CACACAAGTTCTCGAGTCAAG |
| mUcp1 | CTGCCAGGACAGTACCCAAG | GCCACAAACCCTTTGAAAAA |
| mUcp2 | GCCACAAACCCTTTGAAAAA | TACAAGGGGTTCATGCCTTC |
| mUcp3 | AGCCCTCTGCACTGTATGCT | CAGAAAGGAGGGCACAAATC |
| mSREBP1c* | GGCACTAAGTGCCCTCAACCT | GCCACATAGATCTCTGCCAGTGT |
| mSREBP2** | GGATCATCCAGCAGCCTTTGA | ACCGGGACCTGCACCTGT |
| mAcc* | CCCAGCAGAATAAAGCTACTTTGG | TCCTTTTGTGCAACTAGGAACGT |
| mGlut4 | ACTCTTGCCACACAGGCTCT | CCTTGCCCTGTCAGGTATGT |
| mFatp1 | TCAATGTACCAGGAATTACAGAAGG | GAGTGAGAAGTCGCCTGCAC |
| mFatp2 | CATGAGCTAAACCACCAGGG | TTCCTGAGGATACAAGATACCATTG |
| mFatp3 | CGCAGGCTCTGAACCTGG | TCGAAGGTCTCCAGACAGGAG |
| mFatp4 | GCAAGTCCCATCAGCAACTG | GGGGGAAATCACAGCTTCTC |
| mFatp5 | GTGGTCAGAGATTCCAGGTTCC | GCTATACCAGCATGTCCGCTC |
| mFatp6 | TACAACCAAGTGGTGACATCTCTG | AATCTCTTCGGTCAATGGGAC |
| mCd36 | GATGAGCATAGGACATACTTAGATGTG | CACCACTCCAATCCCAAGTAAG |
| mFabp1 | CCAGAAAGGGAAGGACATCA | GTCTCCAGTTCGCACTCCTC |
| mFabp3 | TTCAGCTGGGAATAGAGTTCG | CTGCACATGGATGAGTTTGC |
| mFabp4 | GATGGTGACAAGCTGGTGGT | AATTTCCATCCAGGCCTCTT |
| mFabp5 | GGAAGATGGCGCCTGGTGGA | CCGAGTACAGGTGACATTGT |
| mLpl | CTTCACCCTGCCCGAGG | AACACTGCTGAGTCCTTTCCC |
| mPecam1 | AGAGACGGTCTTGTCGCAGT | TACTGGGCTTCGAGAGCATT |
| mVegfr1 | GGAGGAGTACAACACCACGG | TTGAGGAGCTTTCACCGAAC |
| mMyh6 | AGCTGGAGAATGAGCTGGAG | GCAGCCGCATTAAGTTCTTC |
| mTnnt2 | CGTGAGGAGGAGGAGAACAG | TCCTCTCTGCCAGGATCTTC |
| mVegfr2 | AGCACCTCTCTCGTGATTTCC | AGTAAAAGCAGGGAGTCTGTGG |
| mNrp1 | GGAGCTACTGGGCTGTGAAG | CCTCCTGTGAGCTGGAAGTC |
| CoxII*** | TCTCCCCTCTCTACGCATTCTA | ACGGATTGGAAGTTCTATTGGC |
| Gapdh | TGCGACTTCAACAGCAACTC | GCCTCTCTTGCTCAGTGTCC |

The following primer sequences were taken from these publications:
*Primer sequence from Jiang et al (2005) J Biol Chem 280, 32317-32325
**Primer sequence from Zhou et al (2004) Circ Res 95, 471-478
***Primer sequence from Villena et al. (2007) Proc Natl Acad Sci USA 104, 1418-1423

### EXAMPLE 2

### Role of VEGF-B in Endothelial Targeting of Lipids to Peripheral Tissues

Blood vessels deliver oxygen and nutrients to peripheral tissues. Dietary lipids present in circulation have to be transported through the vascular endothelium in order to be metabolized by tissue cells, a mechanism that has been poorly understood to date. The following examples demonstrate that Vascular Endothelial Growth Factor (VEGF)-B has an unexpected role in endothelial targeting of lipids to peripheral tissues. Bioinformatic analysis showed a tight co-expression of Vegfb and nuclear encoded mitochondrial genes, pointing to a role of VEGF-B in metabolism. VEGF-B specifically controlled the uptake of fatty acids via regulation of fatty acid transport proteins (FATPs) expressed by the endothelium. VEGF-B^{-/-} mice had less accumulation of lipids in their peripheral tissues, and instead accumulated lipids in adipose tissue. The co-expression of VEGF-B and mitochondrial proteins discovered in accordance with the present invention introduces a novel regulatory mechanism, whereby endothelial lipid uptake and mitochondrial lipid utilization by β-oxidation are tightly coordinated. The involvement of a VEGF-family member as a metabolic integrator in peripheral tissues is a novel function for this class of growth factors.

### EXAMPLE 3

### Coordinated Expression of Vegfb and Mitochondrial Genes

Previous studies have shown that Vegfb is highly expressed in metabolically active tissues (Olofsson et al. (1996) Proc Natl Acad Sci USA 93, 2576-2581).

Microarray data from public repositories were used to identify genes that are co-expressed with Vegfb, in order to investigate possible links to established metabolic networks, cellular processes or signaling pathways. A compendium of 708 microarrays was assembled using mouse data from the NCBI GEO database (Barrett et al. (2007) Nucleic Acids Res 35, D760-765). Genes were divided into clusters based on their expression similarity across the arrays in this compendium. Vegfb unexpectedly clustered among a large co-expression group containing nuclear genes coding for components of the respiratory chain (Table 1). All annotated genes in this cluster, apart from Vegfb, encoded mitochondrial proteins. Analysis of expression of Vegfb versus the mean signal of the co-expression cluster generated a Pearson correlation coefficient of r=0.90. A similar analysis for Plgf, Vegfa and Vegfc generated considerably lower correlation coefficients (r≦0.30), as did the myocyte marker Myh7 (r=0.63). In an independent analysis, using the SymAtlas mouse dataset (Su et al. (2002) Proc Natl Acad Sci USA 99, 4465-4470), 31 genes were identified with expression patterns similar to Vegfb. Out of these, 30 genes encoded mitochondrial proteins, mainly components of the respiratory chain, and in particular of respiratory complex I. Identical analyses of Plgf and Vegfa identified 111 and 139 genes respectively, of which none or very few encoded mitochondrial proteins.

To validate the bioinformatic data, the expression of Vegfb, Plgf, Vegfa and two markers for the mitochondrial cluster, Ndufa5 and Cycs, was analyzed in seven mouse tissues by real-time quantitative RT-PCR (qPCR). The data showed that the expression of Vegfb, Ndufa5, and Cycs correlated strongly, with high expression in heart and oxidative skeletal muscle, while Plgf and Vegfa showed different expression profiles. Treatment of cultured C2C12 myotubes, which express VEGFR1, with VEGF-B did not alter the respiratory efficiency of the cells. There was no difference in mitochondrial DNA copy number, or in expression of Ndufa5 and Cycs in hearts from VEGF-B^{-/-} and wild-type (wt) mice, showing that VEGF-B per se does not influence β-oxidation, mitochondrial biogenesis or the expression of the genes in the mitochondrial cluster. The tightly regulated co-expression of Vegfb and transcripts coding for components of the respiratory chain indicated a unique role for VEGF-B in energy metabolism.

### EXAMPLE 4

### VEGF-B^{-/-} Mice have Lower Peripheral Uptake of LCFAs

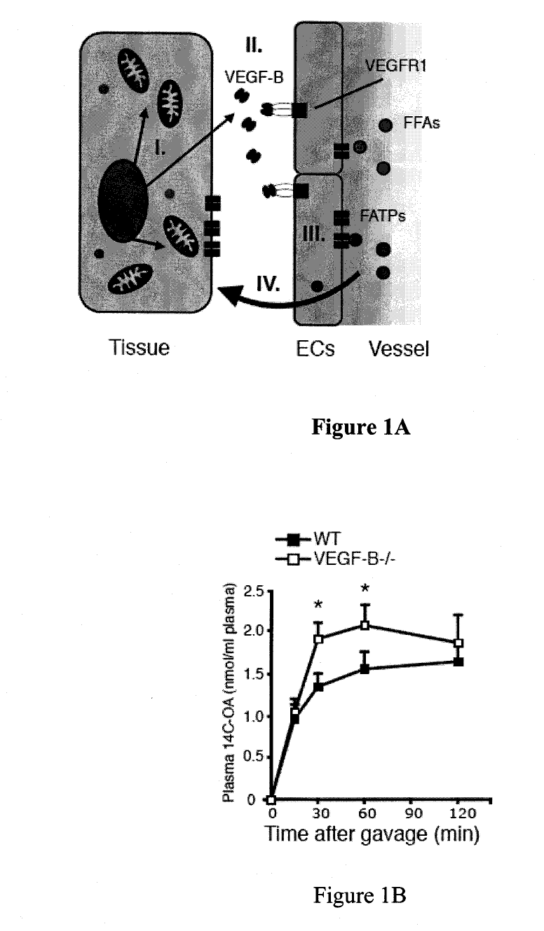

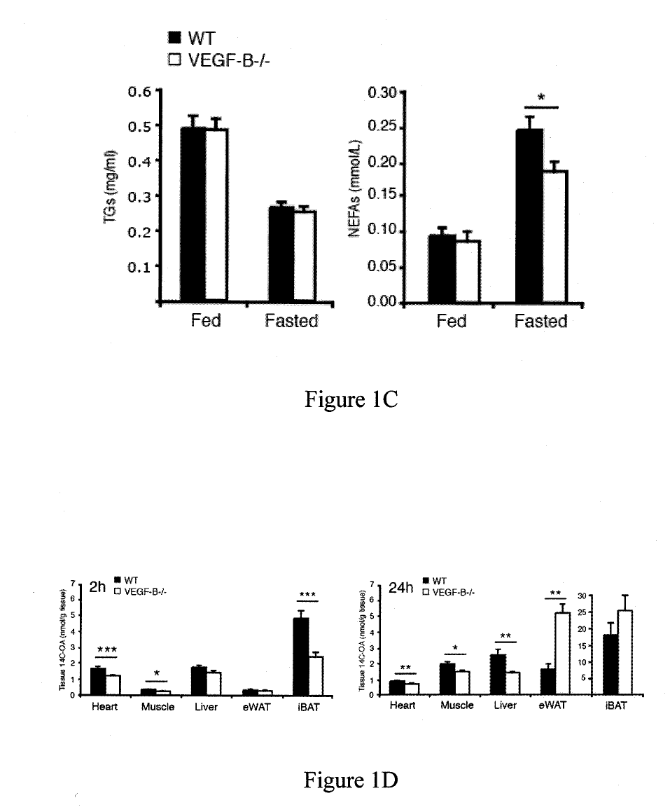

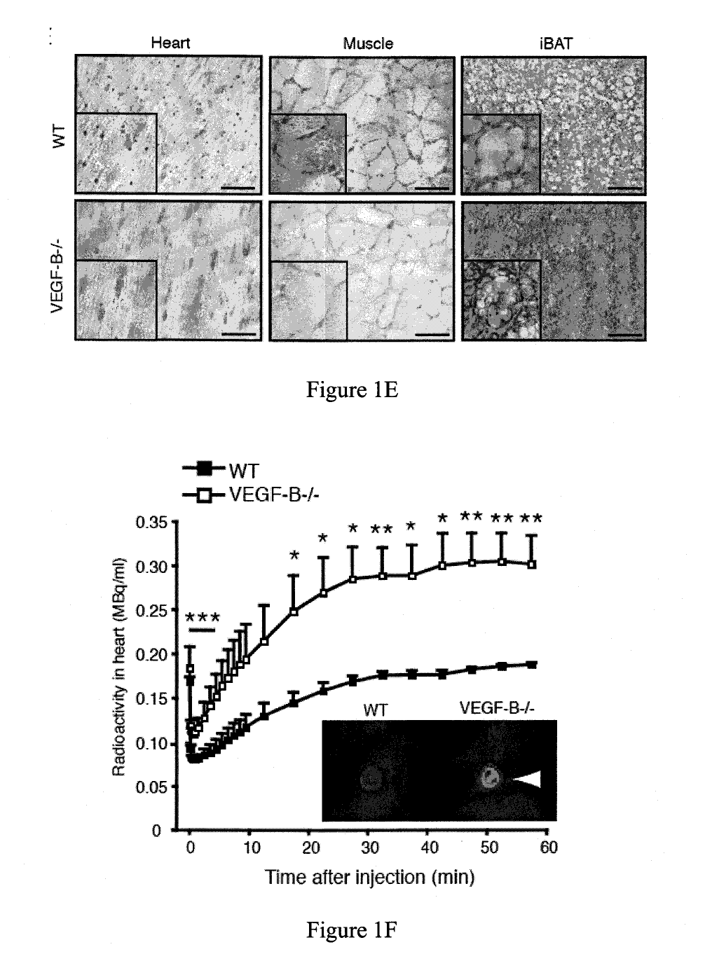

Peripheral tissues with abundant expression of VEGF-B, such as heart, oxidative skeletal muscle and interscapular BAT (iBAT), are known to utilize LCFAs as their main energy source. VEGFR1 is strongly expressed in the vascular endothelium in vivo (Breier et al. (1995) Dev Dyn 204, 228-239). The following example addresses the hypothesis that VEGF-B might effect lipid metabolism by instructing the ECs to take up LCFAs from the circulation, for further transfer to the surrounding tissue for mitochondrial β-oxidation, thus creating a metabolic circuit (FIG. 1A).

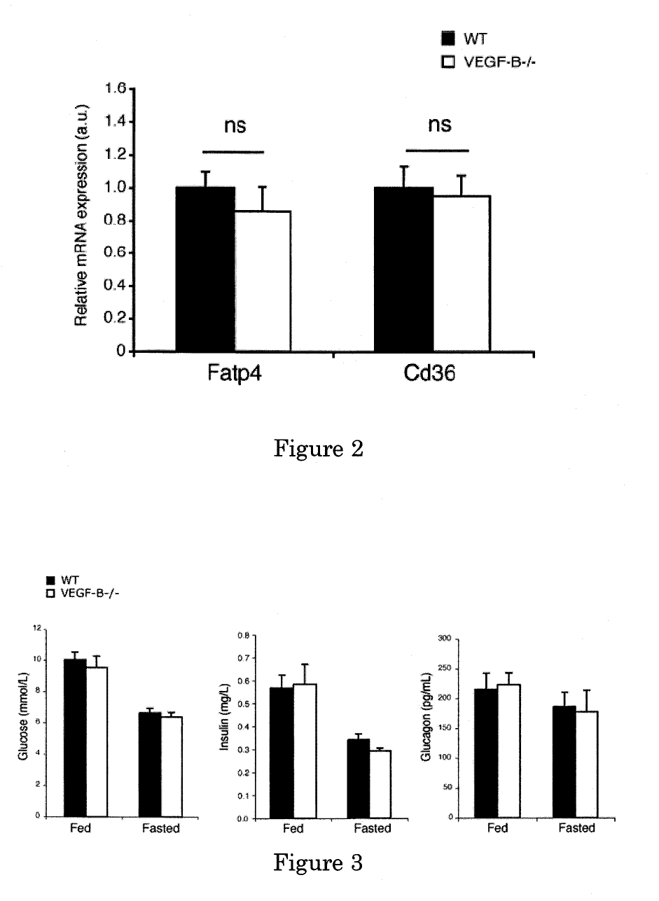

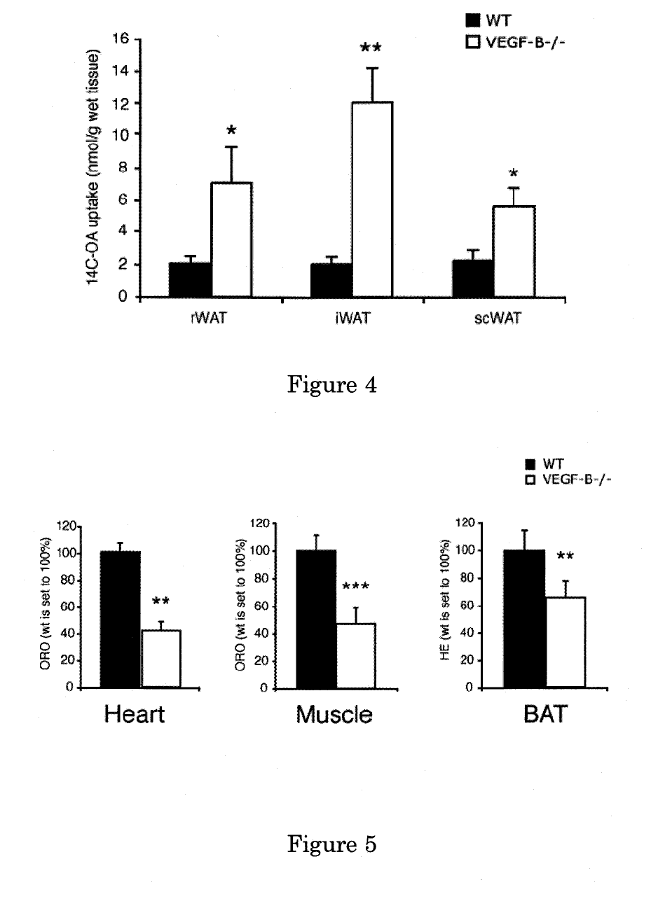

To directly address if VEGF-B^{-/-} mice display changes in LCFA-uptake in peripheral organs, normally fed animals were given radioactively labelled 1-¹⁴C-oleic acid (¹⁴C—OA) by oral gavage, and uptake of the tracer was measured in plasma, and in heart, muscle, liver, epididymal white adipose tissue (eWAT) and iBAT following extensive perfusion. Plasma ¹⁴C-levels were transiently higher in VEGF-B^{-/-} animals compared to wt controls (FIG. 1B). The data further suggested that VEGF-B^{-/-} animals have normal intestinal lipid adsorption since the initial raise in radioactivity was identical between the two genotypes. Animals of both genotypes also expressed equal levels of Cd36 and Fatp4, the major intestinal fatty acid handling proteins, in their small intestines (FIG. 2) (Nauli et al. (2006) Gastroenterology 131, 1197-1207; Stahl et al. (1999) Mol Cell 4, 299-308). A basal difference in plasma lipids was not the cause of the genotype-associated differences in clearance of ¹⁴C—OA from plasma, as wt and VEGF-B^{-/-} animals had similar plasma levels of triglycerides (TGs) and non-esterified fatty acids (NEFAs) in the fed state (FIG. 1C). Fasted VEGF-B^{-/-} mice had lower plasma NEFAs, indicating less fatty acid release from WAT after fasting. Plasma glucose, insulin and glucagon were unaltered both in the fed and fasted state (FIG. 3).

Measuring the uptake of the LCFA-tracer in wt and VEGF-B^{-/-} organs 2 hrs after oral gavage showed that significantly less ¹⁴C—OA accumulated in VEGF-B^{-/-} heart, muscle, and iBAT as compared to wt organs (FIG. 1D, left panel), and the difference was already detectable 30 minutes post-gavage (data not shown). Extending the post-gavage period to 24 firs revealed a significantly higher accumulation of ¹⁴C—OA in VEGF-B^{-/-} WAT-pads as compared to wt animals, whereas less ¹⁴C—OA was detected in VEGF-B^{-/-} heart, muscle and liver (FIG. 1D right panel, and 4). There was no detectable difference in ¹⁴C—OA in iBAT between the two genotypes after 24 hrs. These results showed that VEGF-B^{-/-} mice have a defect in uptake of LCFAs into their peripheral organs, and instead accumulate excess lipids in various fat pads.

LCFAs taken up by peripheral organs, that are not catabolised by mitochondria or peroxisomes, are stored in cells as neutral lipid droplets. Accumulation of these neutral lipids was analyzed by oil red O (ORO) staining of tissue sections from heart and oxidative muscle, or haematoxylin-eosin (HE) staining of iBAT (FIG. 1E). The results showed a striking difference between the genotypes with smaller and less abundant intracellular lipid droplets and vacuoles in all examined VEGF-B^{-/-} tissues. Computer-assisted quantitation showed a 60% reduction in lipid staining in VEGF-B^{-/-} heart and muscle, and 40% smaller lipid vacuoles in iBAT (FIG. 5). Analysis of the expression level of a large number of regulators and enzymes in fatty acid metabolism showed only minor expressional changes (Table 2), including reduced expression of Cpt1 and slightly increased expression of Srebps. The analysis therefore indicated an induction of lipogenesis instead of β-oxidation, and showed that the genotype-associated differences in lipid staining was not due to higher β-oxidation in VEGF-B^{-/-} organs.

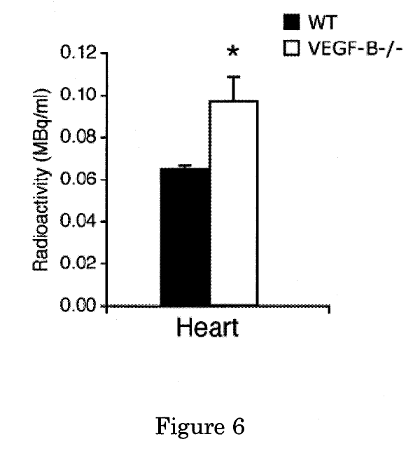

Reduced intra-myocellular lipid accumulation has been proposed to enhance glucose utilization and insulin sensitivity of myocytes (Peterson et al (2002) J R Soc Med 95 Suppl 42, 8-13: Savage et al (2007) Physiol Rev 87, 507-520). To explore whether the lower lipid uptake, due to VEGF-B deficiency, resulted in increased glucose uptake to the heart, the uptake of ¹⁸F-deoxyglucose ([¹⁸F]FDG) was analyzed by positron emission tomography (PET). VEGF-B^{-/-} hearts accumulated significantly more [¹⁸F]FDG compared to wt controls (FIG. 1F), and direct measurements of the radioactivity in dissected hearts confirmed the results from image analysis (FIG. 6). The expression level of Glut4 was also increased in VEGF-B^{-/-} hearts compared to wt hearts (Table 2). Taken together, the molecular machinery for accumulation of [¹⁸F]FDG from the blood was more active in the VEGF-B^{-/-} hearts as compared to controls, indicating a compensatory increase in carbohydrate utilization for energy production.

### EXAMPLE 5

### VEGF-B^{-/-} Mice have a Complex Metabolic Phenotype Including Increased Ketosis, Accumulation of WAT and Lower Plasma Leptin

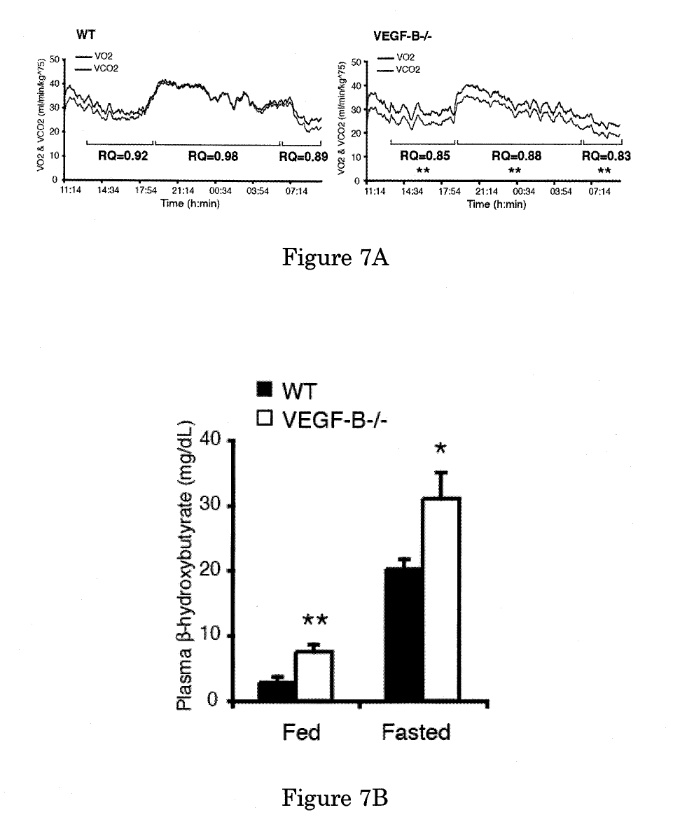

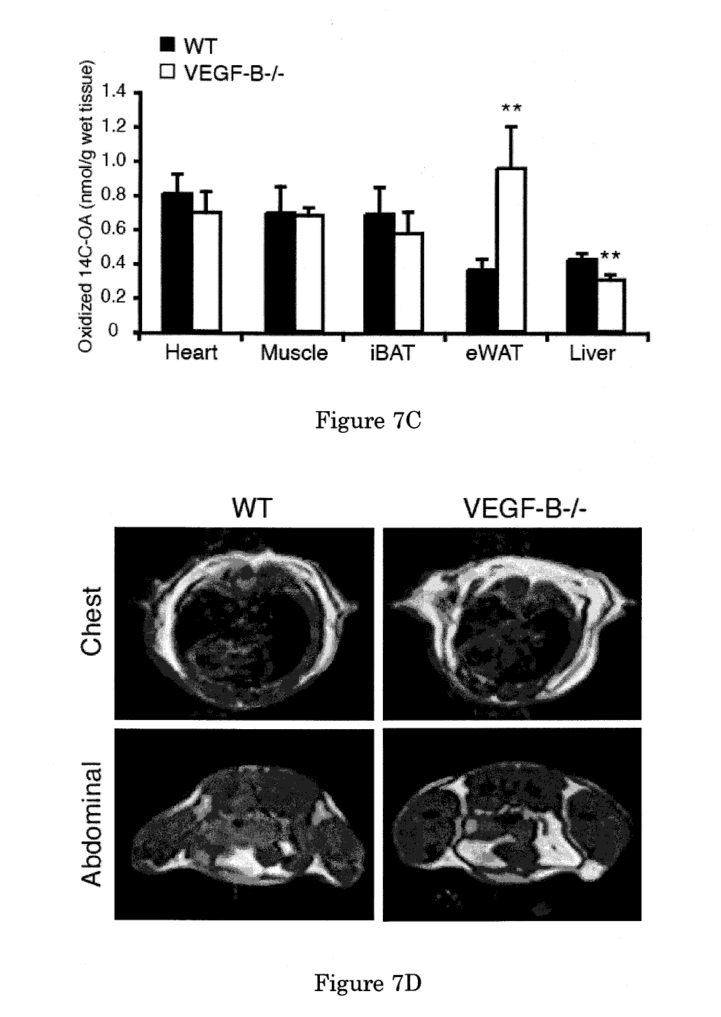

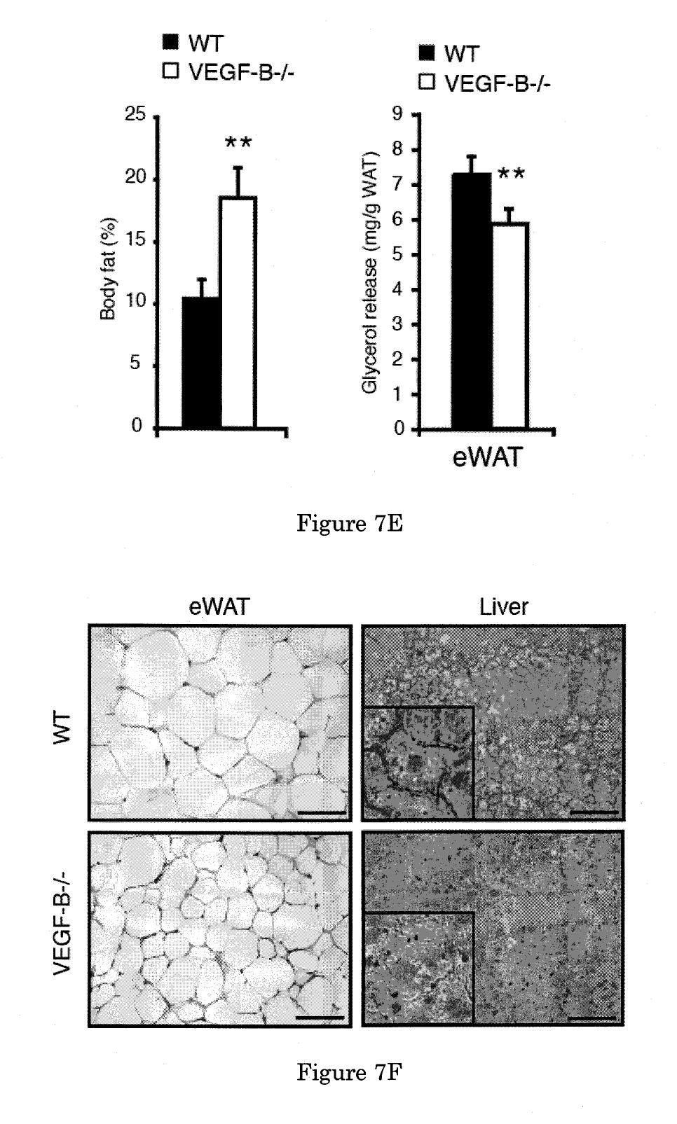

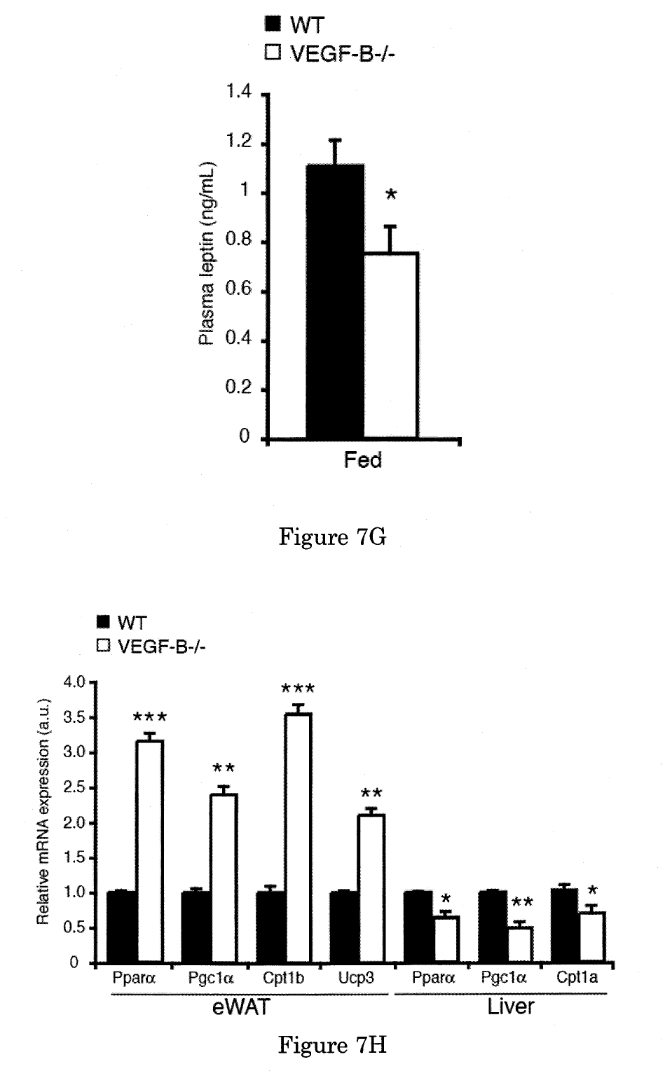

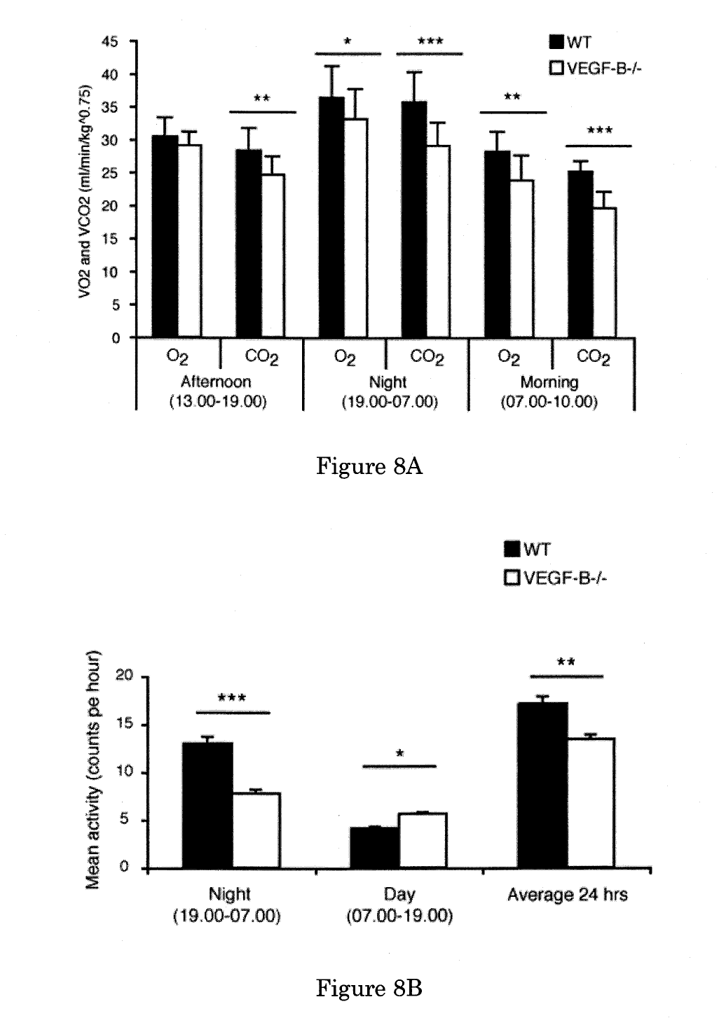

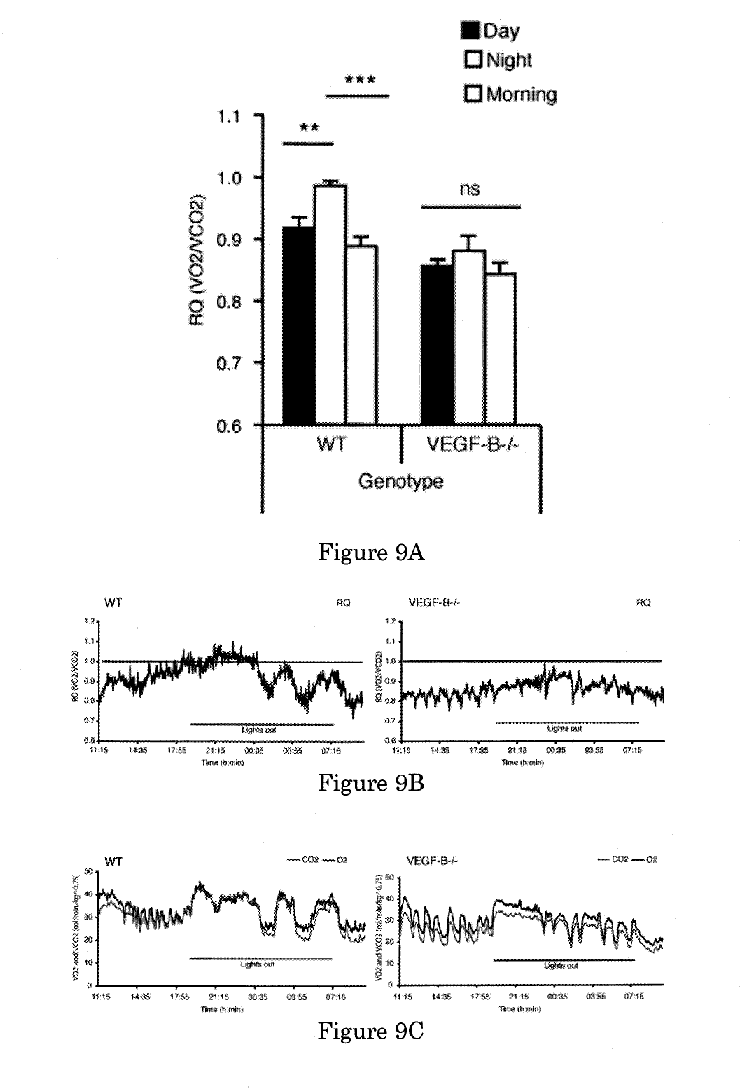

Because VEGF-B^{-/-} heart, oxidative muscle and BAT, which normally metabolize LCFAs, had lower uptake of ¹⁴C—OA, the secondary effects on whole body metabolism were examined. 18-week-old VEGF-B^{-/-} and control animals were analyzed using indirect calorimetry, measuring VO₂ and VCO₂ during 23 hrs. The VO₂ utilization and the respiratory quotient (RQ) were lower for the VEGF-B^{-/-} animals (FIGS. 7 and 8A), yielding a lower metabolic rate (VEGF-B^{-/-} 6.72±0.15 kcal/h/kg, wt 7.44±0.11 kcal/h/kg, n=6-7, p<0.01). The VEGF-B^{-/-} mice moreover had reduced voluntary activity during night time (FIG. 8B), in line with the mice having similar RQ values and oxygen usage patterns during all times of the 23-hour indirect calorimetry analysis (FIG. 9A-C). Taken together, the results indicated that VEGF-B^{-/-} mice have a less adaptable metabolism as compared to wt mice.

A low RQ indicates either direct lipid oxidation by muscles and heart, or in the fasted state, a conversion of LCFAs to ketone bodies in liver or kidney, and subsequent transport of the ketones to various organs for further oxidation. Significantly higher levels of β-hydroxybutyrate, a marker for increased ketosis, in plasma from the VEGF-B^{-/-} mice, both in the fed state and after fasting (FIG. 7B). Performing ex-vivo ¹⁴C—OA β-oxidation assays confirmed that LCFA oxidation was unaltered in VEGF-B^{-/-} heart, oxidative muscle and iBAT, which means that the lower RQ values of the VEGF-B^{-/-} mice could not be a results of increased β-oxidation of LCFAs in these organs, and probably is due to ketosis (FIG. 7C).

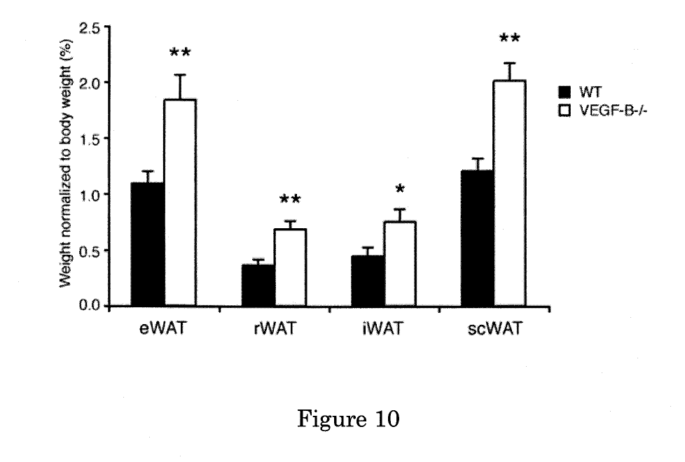

In vivo lipid uptake experiment and plasma analyses (FIG. 1C-D) both indicated that VEGF-B^{-/-} mice accumulate excess lipids in their fat pads due to increased lipid uptake and decreased fatty acid release. Accordingly, it was found that 16-18 week old male VEGF-B^{-/-} mice weighted approximately 15% more as compared to matched wt controls (VEGF-B^{-/-} 29.9±1.5 g, wt 26.1±1.0 g, n=11-15, p<0.001). This weight difference developed with increasing age, and was not detectable in young mice (<12 weeks) (Aase et al. (2001) Circulation 104, 358-364; Bellomo et al. (2000) Circ Res 86, E29-35). Using magnetic resonance imaging (MRI) it was demonstrated that VEGF-B^{-/-} animals have higher body fat mass (FIG. 7D), and that fat pads dissected from VEGF-B^{-/-} mice are 60-90% larger then control pads (FIG. 10). It was confirmed that VEGF-B^{-/-} WAT pads have reduced lipolysis by measuring glycerol release ex vivo (FIG. 7E), but in contrast qPCR analysis of WAT mRNA showed no change in hormone sensitive lipase (Hsl) expression (Table 2). This could be due to post-transcriptional regulation of the enzyme activity. Lipoprotein lipase (Lpl) expression was somewhat upregulated, in line with increased uptake of lipids to VEGF-B^{-/-} WAT (Table 2). Taken together, the accumulation of lipids in adipose tissue of the VEGF-B^{-/-} mice instead of in peripheral tissues is due to multiple mechanisms: shunting of the unabsorbed lipids to WAT, reduced lipolysis and NEFA release from WAT, and an overall lower metabolic rate.

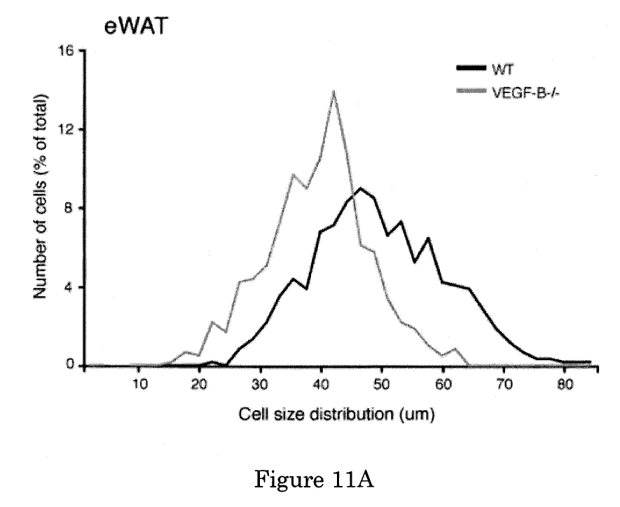

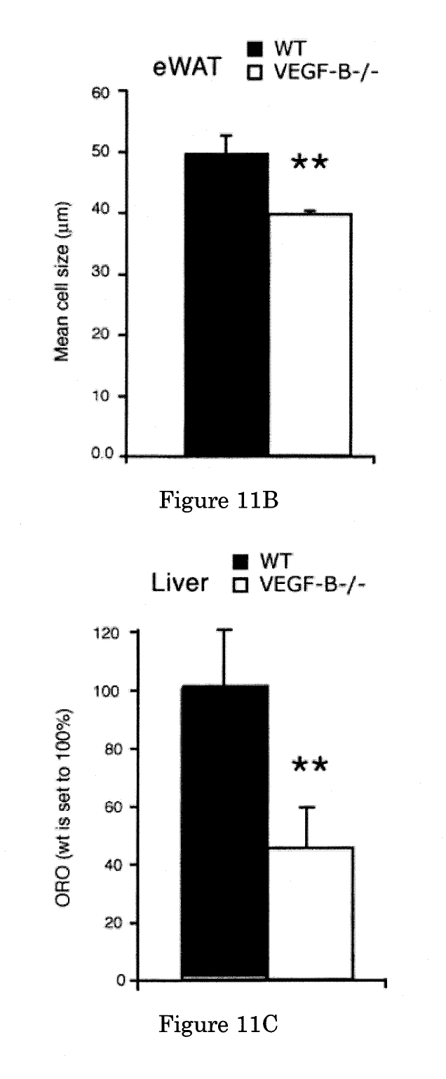

The WAT and liver are lipid-buffering organs that function to maintain whole body lipid homeostasis. The incorporation of TGs and neutral lipids was analyzed in VEGF-B^{-/-} WAT and liver by HE and ORO staining, respectively. VEGF-B^{-/-} mice had small hyperplastic adipocytes instead of the expected large hypertrophic fat cells that are typical of decreased lipolysis and increased lipid uptake (FIGS. 7F and 11). ORO staining of liver similarly showed less abundant lipid droplets in VEGF-B^{-/-} animals. Adipocytes secrete several adipokines, including leptin, in relationship to adipocyte cell size and WAT mass (Skurk et al. (2007) J Clin Endocrinol Metab 92, 1023-1033). Leptin regulates feeding behaviour by acting on the hypothalamus, but also strongly affects both WAT and liver lipid homeostasis and metabolism through its central hypothalamic actions (Holness (2007) Endocrinology 148, 5601-560). It was found that VEGF-B^{-/-} mice, in line with their smaller adipocytes, had significantly lower plasma leptin levels as compared to wt mice (FIG. 7G). qPCR analysis of the expression of PPARα and other metabolic genes in WAT and liver showed upregulation of most genes in VEGF-B^{-/-} WAT and conversely downregulation in liver (FIG. 7H and Table 22). This is in agreement with the known organ-specific influence of leptin on PPARα expression through its hypothalamus mediated actions (Gallardo et al. (2007) Endocrinology 148, 5604-5610). In line with the altered PPARα expression, the ex-vivo β-oxidation assays demonstrated that VEGF-B^{-/-} WAT had a higher metabolic rate then controls (FIG. 7C), whereas VEGF-B^{-/-} livers had a lower oxidative capacity.

In summary, VEGF-B deficiency leads primarily to lower peripheral LCFA-uptake and lipid accumulation in organs where VEGF-B normally is expressed at high levels. This LCFA-uptake defect induces a number of secondary alternations on whole body metabolism, including increased ketone and glucose utilization, lower plasma leptin levels, lower metabolic activity and increased weight gain.

### EXAMPLE 6

### VEGF-B^{-/-} Animals have Reduced Expression of Fatty Acid Transport Proteins

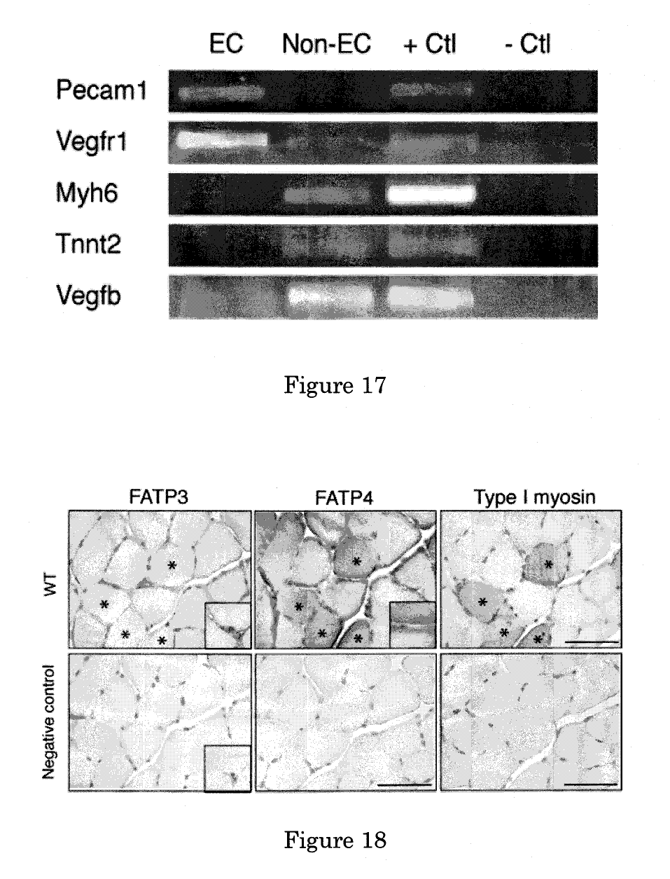

Dietary LCFAs are transported in plasma as albumin-bound NEFAs, or as lipoprotein-associated TGs that are hydrolysed by LPL upon reaching their target organs. The subsequent transcytosis of LCFAs across the endothelium is however poorly characterized. The hypothesis that the molecular mechanism behind the LCFA-uptake defect of the VEGF-B^{-/-} mice is due to changes in lipid handling proteins expressed by ECs, as these cells, but not cardiomyocytes or myocytes, express VEGFR1 in vivo (FIGS. 2A and 17) (Breier et al. (1995) Dev Dyn 204, 228-239) was investigated. Wt and VEGF-B^{-/-} organs were analyzed for mRNA expression levels of proteins that previously have been shown to facilitate LCFA-uptake, including the six Fatty Acid Transport Proteins (FATPs), the Fatty Acid Binding Proteins (FABPs), CD36 and LPL (Stahl (2004) Pflugers Arch 447, 722-727; van der Vusse (2000) Cardiovasc Res 45, 279-293).

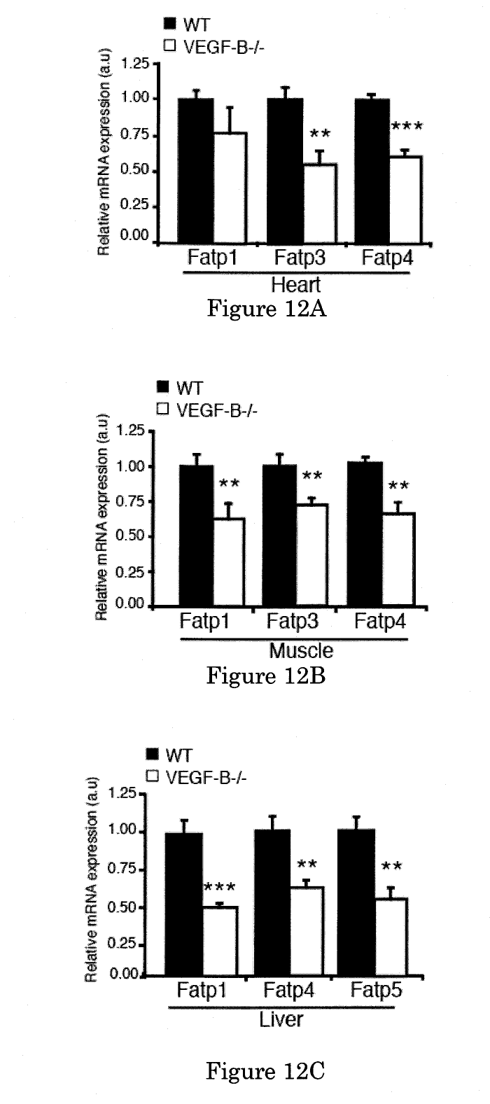

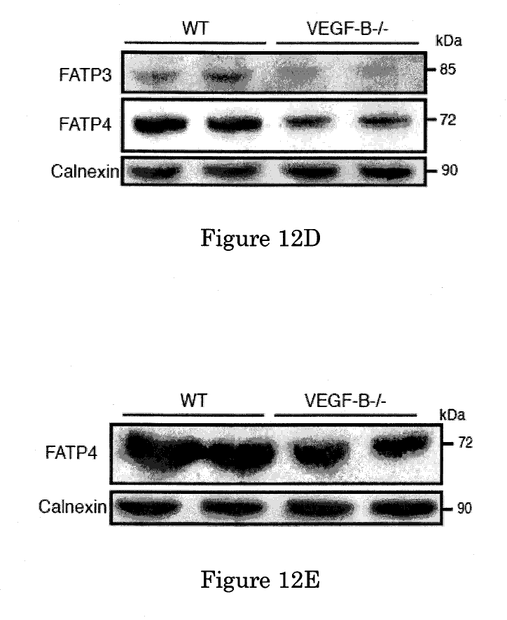

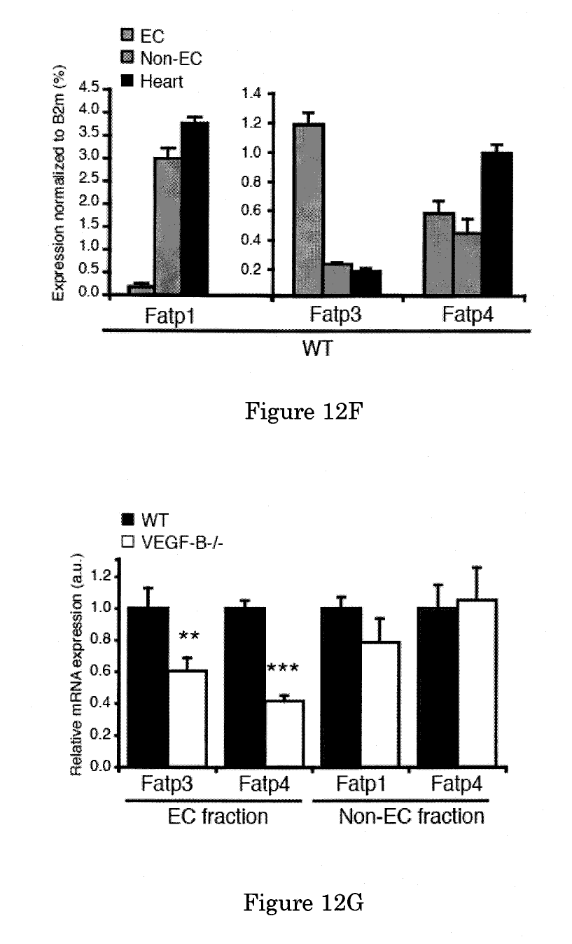

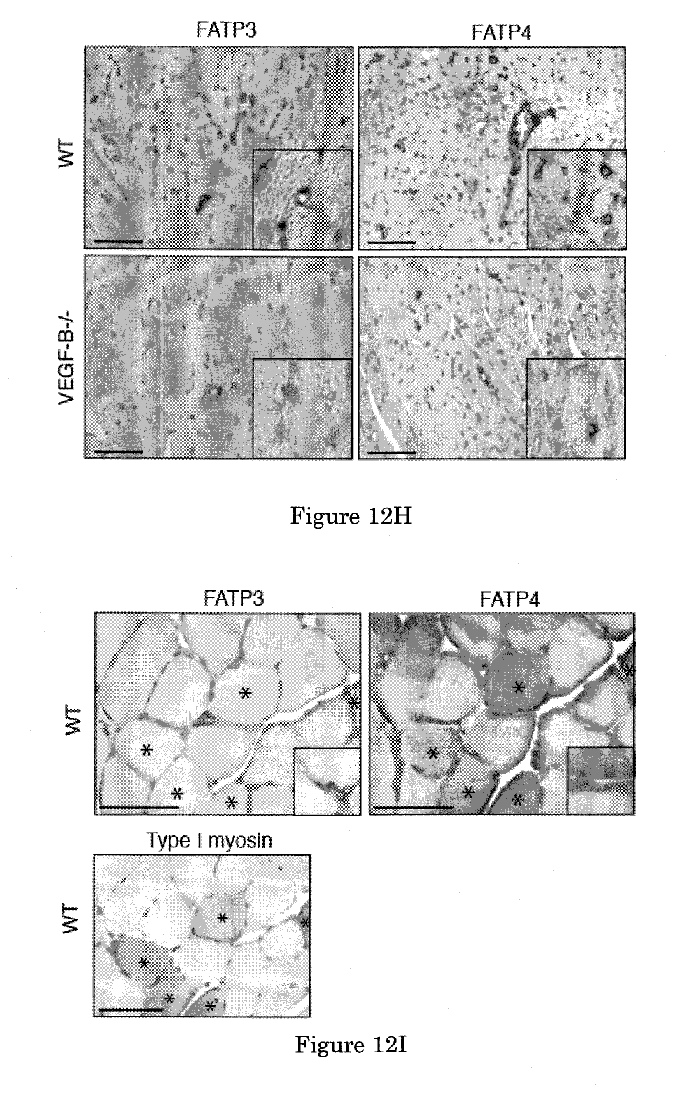

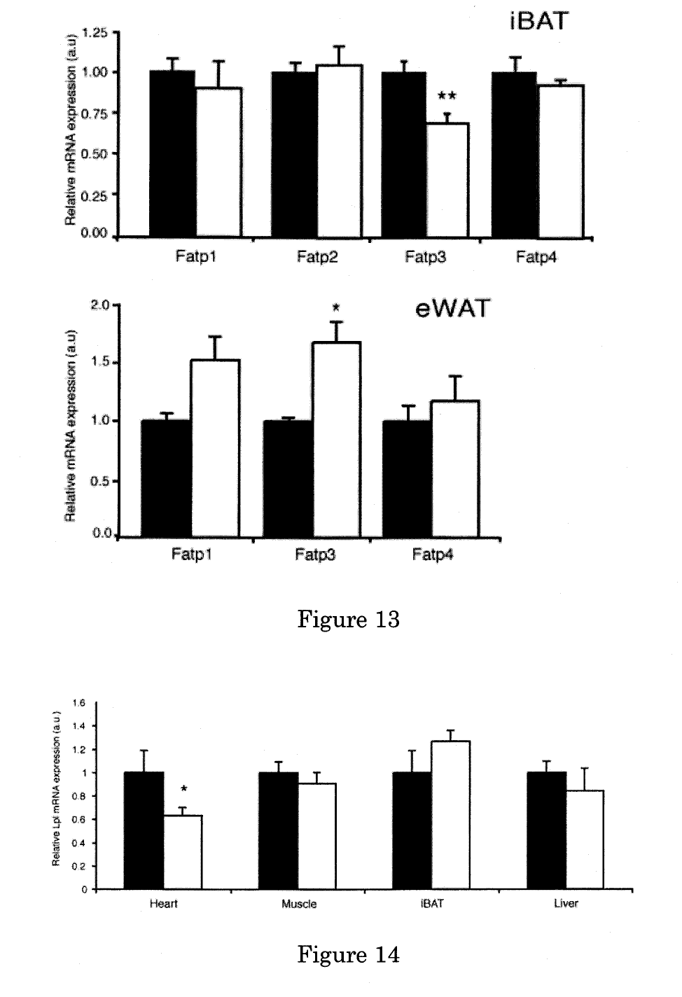

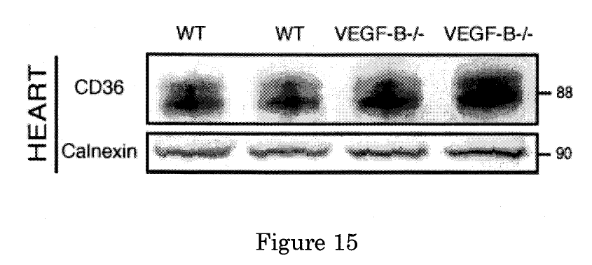

Expression analysis of these transcripts revealed a downregulation of several FATP mRNAs in most peripheral VEGF-B^{-/-} tissues analyzed except eWAT (FIGS. 12 and 13). Similar analysis of the mRNA expression of Lpl showed reduced expression in VEGF-B^{-/-} hearts, but not in VEGF-B^{-/-} muscle, iBAT, liver or eWAT (FIG. 14 and Table 2). The expression levels of FABPs and Cd36 showed no genotype-associated alterations in any organ analyzed (data not shown). Immunoblotting of tissue lysates from wt and VEGF-B^{-/-} animals confirmed the reduced expression of FATPs in heart (FIG. 12D: FATP3, 38% of wt levels set to 100%, p<0.05; FATP4, 42%, p<0.05) and liver (FIG. 12E: FATP4, 58%, p<0.05). CD36 protein levels were higher in VEGF-B^{-/-} hearts as compared to controls, excluding CD36 as the mediator of the VEGF-B dependent effect on LCFA-uptake (FIG. 15).

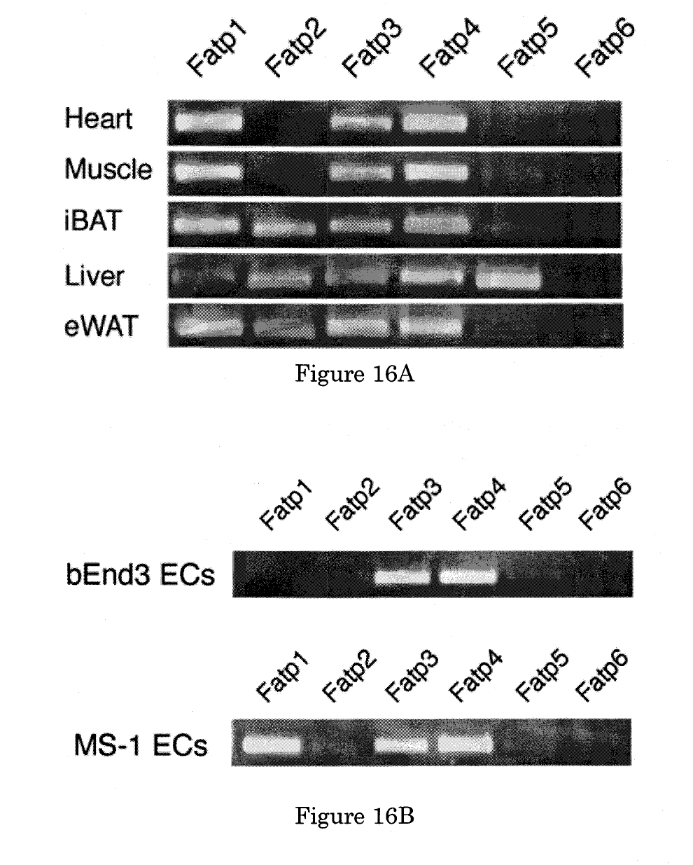

It had not previously been established which of the six FATPs are expressed by ECs, or if ECs express any FATPs at all (Stahl (2004) Pflugers Arch 447, 722-727). It was determined herein that Fatp1, Fatp3 and Fatp4 were ubiquitously expressed in all mouse tissues analyzed while Fatp2, 5 and 6 were expressed in a more tissue-restricted fashion (FIG. 16A). Similarly, two cultured mouse EC lines, bEnd3 and MS-1, expressed combinations of Fatp1, Fatp3 and Fatp4 (FIG. 16B). Endothelial cells were isolated from hearts of wt and VEGF-B^{-/-} animals to analyze whether FATPs are expressed in the endothelium in vivo, and if the endothelial FATPs are transcriptionally regulated by VEGF-B. RT-PCR analyses of marker genes for the EC-fraction and non-EC fraction showed that the isolation procedure generated EC-fractions highly enriched in ECs (FIG. 17). In wt hearts, Fatp1 was mostly expressed in the non-EC fraction, Fatp3 in the EC fraction, while Fatp4 was expressed in both (FIG. 12F). When comparing the expression of the FATPs in cell fractions isolated from animals of both genotypes, it was observed that both Fatp3 and Fatp4 were significantly downregulated in the VEGF-B^{-/-} EC-fractions, while expression of FATPs was unchanged in the non-EC fractions (FIG. 12G).

The vascular expression of FATP3 and FATP4 was confirmed by immunohistochemistry. In wt mouse hearts, both FATPs were highly expressed in capillaries and some larger vessels (FIG. 12H), with weaker staining in VEGF-B^{-/-} hearts. In addition, staining for FATP4, but not for FATP3, was also detected in cardiomyocytes of both genotypes. Similarly in skeletal muscle, FATP3 was only expressed by ECs, while FATP4 was also detected in muscle fibres (FIGS. 12I and 18). The highest FATP4 expression was found in type I fibres, as confirmed by staining for the marker protein type I myosin. It was concluded that FATP3 is an endothelial-specific FATP, and that VEGF-B controls the expression of endothelial Fatp3 and Fatp4 in several metabolically active peripheral tissues.

### EXAMPLE 7

### VEGF-B Signaling Induces FATP Expression in Vivo and in Vitro

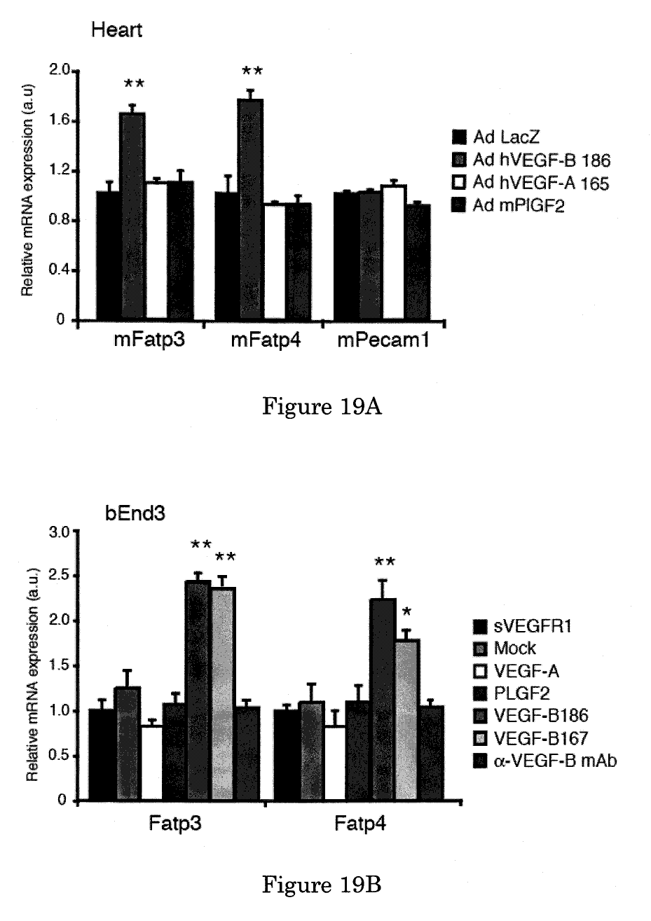

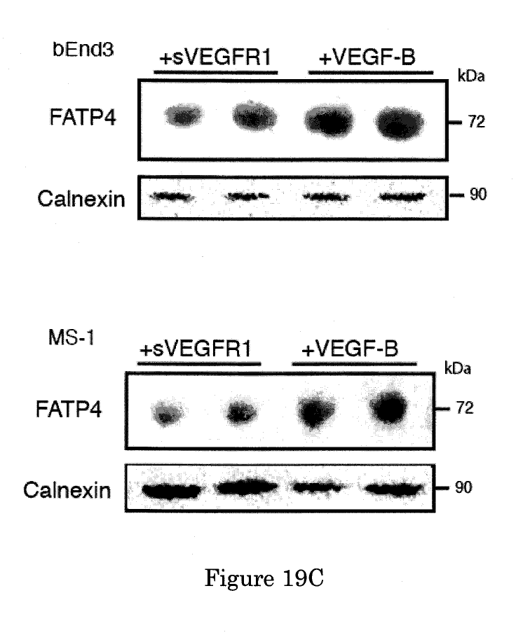

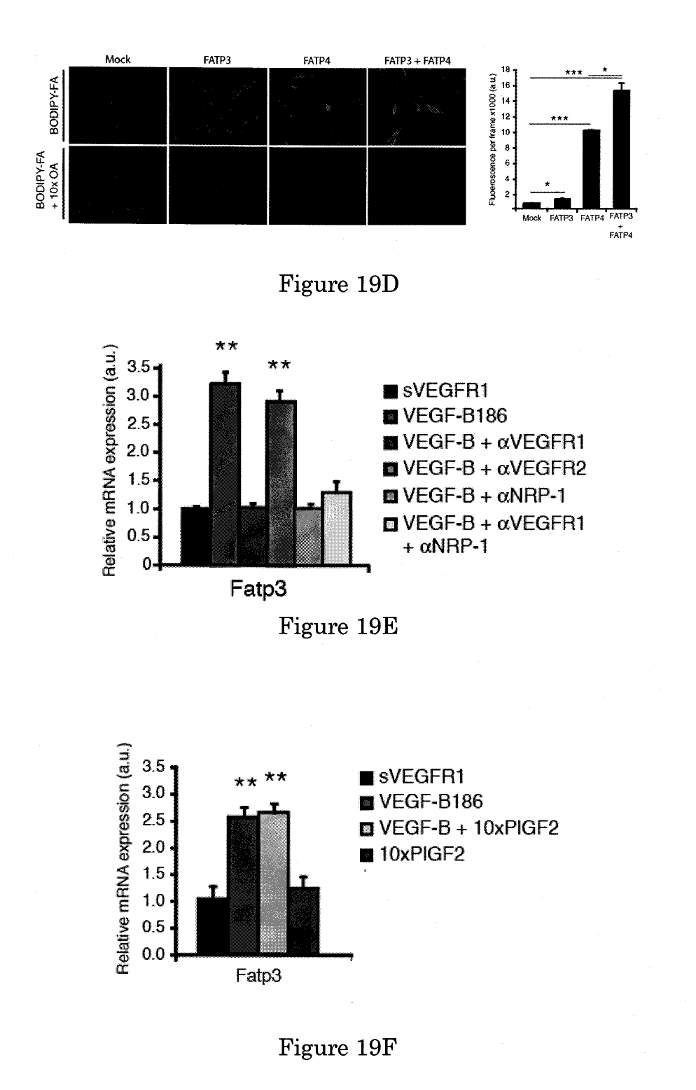

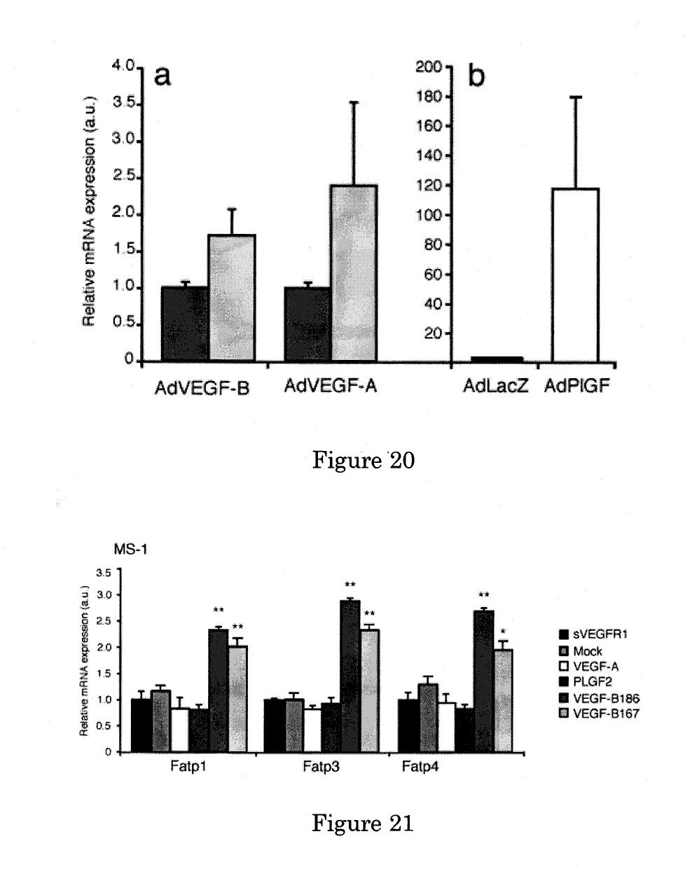

To analyze whether VEGF-B could induce FATP and possibly also LPL expression in hearts in vivo, adenoviruses (Ad) encoding hVEGF-B₁₈₆, hVEGF-A₁₆₅, mPlGF2 or LacZ were directly injected into mouse hearts by echo-guided ventricular injections. After six days, the hearts were recovered and mRNA expression of Fatp3, Fatp4, Lpl, h VEGFB, h VEGFA and total mPlgf was determined by qPCR (FIGS. 19A and 20). Strikingly, AdVEGF-B transduced hearts displayed a 50% increase in Fatp3 and Fatp4 mRNA expression that was not detected in AdVEGF-A, AdPlGF or AdLacZ transduced tissues. There was no change in Lpl expression in any of the transduced hearts (data not shown). The transcriptional induction of FATPs was not due to proliferation of ECs as expression of the endothelial marker Pecam1 was not increased (FIG. 19A). Taken together, the data showed that VEGF-B does regulate the expression of the vascular FATPs in vivo, but not that of Lpl.

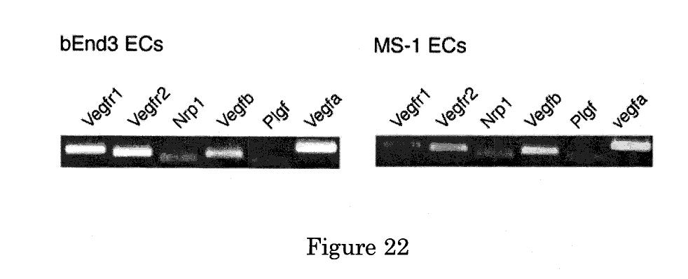

The role of the FATPs in EC-mediated lipid uptake was further characterized. To examine whether VEGF-B could modulate FATP expression in ECs in vitro, bEnd3 and MS-1 cells were treated with different VEGFs before transcript analysis (FIGS. 19B and 21). Both cell lines expressed the receptors Vegfr1 and 2, Nrp1, as well as Vegfb, Plgf and Vegfa (FIG. 22), and therefore control cultures were treated with soluble VEGFR1 (sVEGFR1), or a neutralizing anti-VEGF-B mAb. Analysis by qPCR revealed that treatment with both VEGF-B-isoforms increased the expression of the endothelial FATPs 2-3 fold, with the more diffusible VEGF-B₁₈₆ isoform being somewhat more efficient. Other VEGFR1 ligands could not induce FATP expression in either cell line, and VEGF-B treatment did not alter the expression of any FABPs analyzed (data not shown). Cd36 was not expressed by any of the EC lines (data not shown). The VEGF-B₁₈₆ mediated induction of FATP4 protein expression in both EC lines was confirmed by immunoblotting (FIG. 19C, in bEnd3 cells 2.1-fold versus sVEGFR1 levels set to 1 unit, p<0.05; in MS-1 cells 3.5-fold, p<0.05).

The bEnd3 cells were transfected with expression plasmids encoding FATP3 or FATP4, or with mock control (FIG. 19D), and computer-assisted quantitation of cellular uptake of a fluorescent BODIPY-labelled LCFA (BODIPY-FA) was performed. FATP3 transfection led to increased cellular BODIPY-FA uptake (1.8±0.3 fold). FATP4 was much more potent in inducing cellular BODIPY-FA uptake (14±0.1 fold), whereas the co-expression of both FATPs resulted in the highest accumulation of BODIPY-FA, suggesting a synergistic effect (21±1.4 fold). Adding a 10× molar excess of unlabeled oleic acid (OA) to the uptake assay abolished cellular BODIPY-FA uptake, demonstrating a saturable uptake mechanism.

To verify that the effect of VEGF-B is mediated through the endothelial VEGFR1, and/or NRP-1, bEnd3 cells were pre-incubated with neutralizing antibodies against either receptor before adding VEGF-B₁₈₆. Analysis of FATP expression showed that both VEGFR1 and NRP-1 were essential for VEGF-B mediated signal transduction, whereas a neutralizing VEGFR2 antibody had no effect (FIG. 19E). It was further explored whether excess PlGF would block the effect of VEGF-B. The results showed that a 10-fold molar excess of PlGF2 did not prevent VEGF-B induced expression of the FATPs (FIG. 19F). In conclusion, the data from the in vitro stimulations show that VEGF-B specifically induced expression of endothelial FATPs in a VEGFR1 and NRP-1 dependent manner, and that expression of FATP3 and FATP4 in ECs induced cellular LCFA-uptake.

### EXAMPLE 8

### VEGF-B Controls Trans-Endothelial LCFA Transport by Controlling FATP Expression

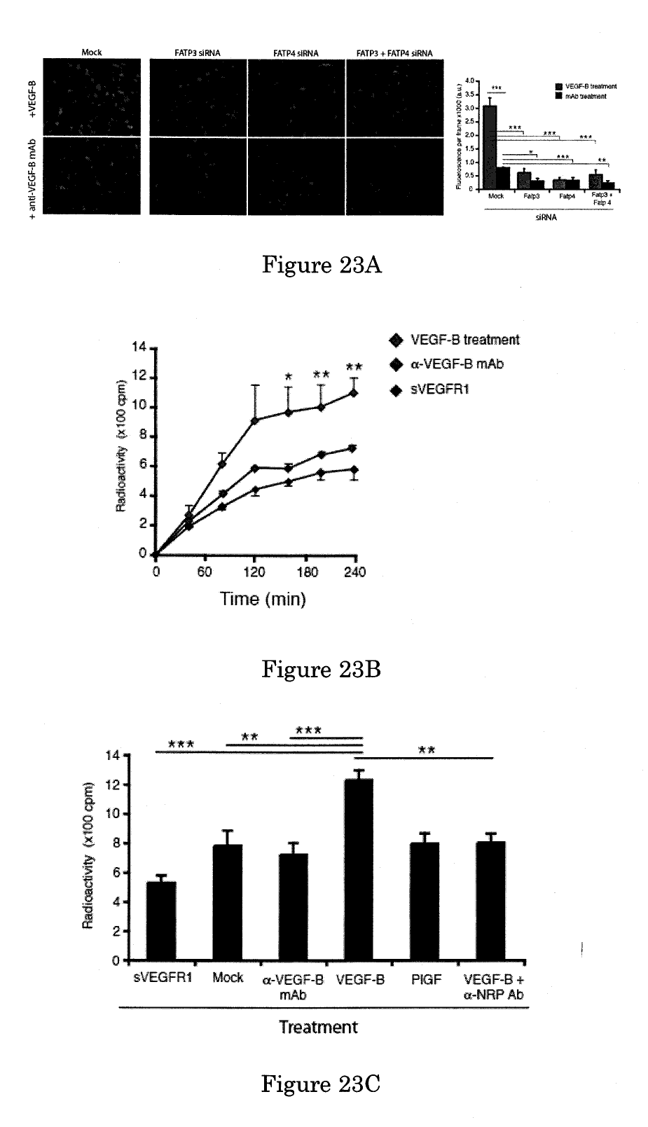

To determine whether VEGF-B per se induces higher cellular LCFA-uptake to ECs via its transcriptional induction of the FATPs, bEnd3 cells were treated with VEGF-B₁₈₆, or with anti-VEGF-B mAb, and LCFA-uptake was assessed using BODIPY-FA (FIG. 23A, 1^{st} column). VEGF-B₁₈₆ treatment induced significantly higher BODIPY-FA uptake into cells (3.9±0.4 fold) compared to the control.

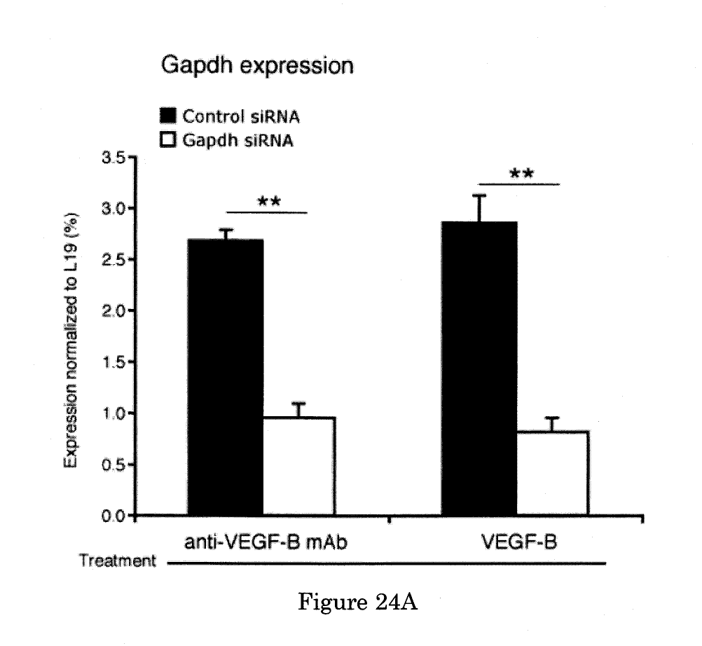

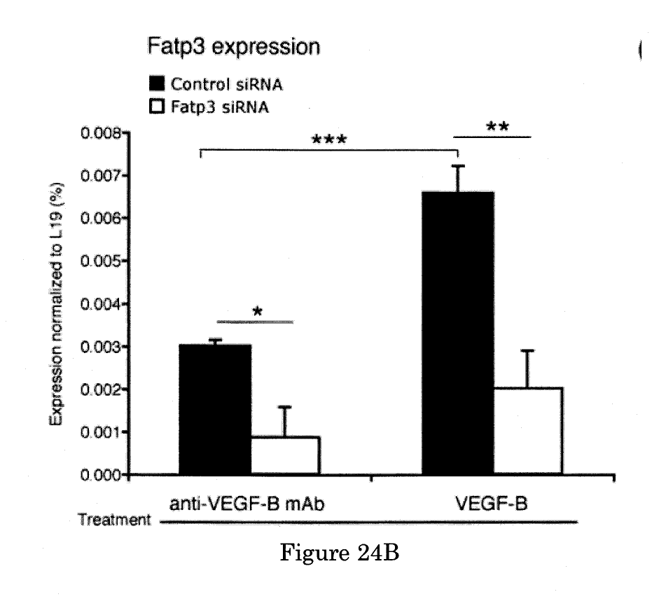

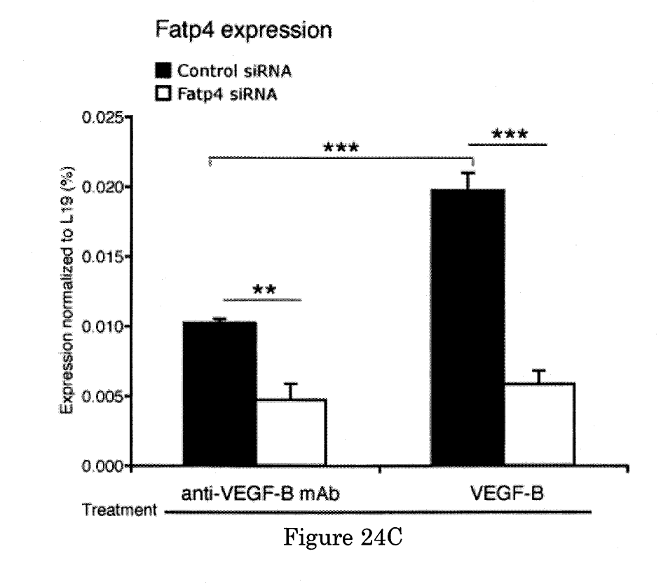

To establish that the FATPs mediated the VEGF-B induced increase in BODIPY-FA uptake, small interfering RNAs (siRNAs) were used to specifically silence Fatp3 or Fatp4 expression (FIG. 23A, 2^{nd}-4^{th} column). After siRNA transfection, the cells were treated with VEGF-B₁₈₆, or with anti-VEGF-B mAb, and their capacity to accumulate BODIPY-FA was assessed. The results showed that silencing of FATP3, FATP4 or both FATPs efficiently reduced the capacity of the cells to accumulate BODIPY-FA, and blunted the effect of VEGF-B treatment. Mock transfection or control siRNA (scrambled siRNA or siRNA targeting Gapdh) had no effect on the cellular lipid uptake (data not shown). The efficiency of the knock down was confirmed by qPCR (FIG. 24).

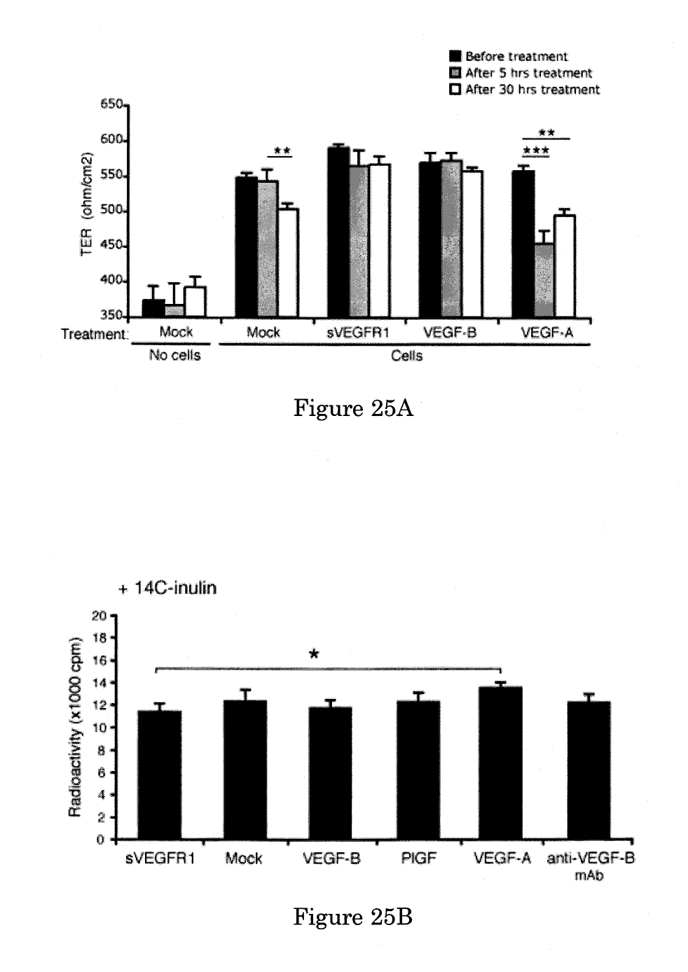

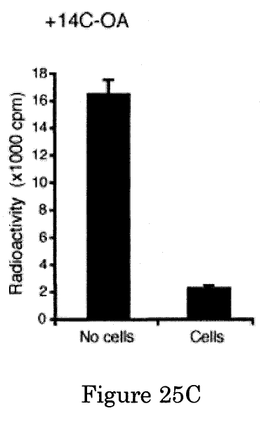

The bEnd3 cells were cultured on cell culture insert membranes, creating two liquid compartments separated by a tight monolayer of cells, thus mimicking the endothelium of blood vessels. VEGF-B treatment of the monolayers did not reduce trans-endothelial resistance (FIG. 25A). Similar results were obtained from control experiments monitoring the integrity of the monolayers in response to different VEGFs by measuring trans-endothelial leakage of ¹⁴C-inulin, an inert carbohydrate tracer (FIG. 25B). It was further found that untreated bEnd3 monolayers efficiently prevented leakage of the previously described radioactive LCFA, ¹⁴C—OA, from the apical to the basal compartment (FIG. 25C).

Kinetic studies of trans-endothelial transfer of ¹⁴C—OA showed that VEGF-B treatment significantly increased ¹⁴C—OA transcytosis (FIG. 23B). Comparing different VEGF treatments showed that increased transfer of the ¹⁴C-LCFA through the ECs was only observed in cultures treated with VEGF-B (FIG. 23C), and treating the monolayers with a neutralizing anti-NRP1 antibody in combination with VEGF-B abolished this transport. VEGF-B thus has a unique role in regulation of trans-endothelial transport of LCFAs (FIG. 1A).

### EXAMPLE 9

### Role of VEGF-B in the Pathology of Type 2 Diabetes (T2DM)

Expression of mitochondrial genes and Vegfb in hearts from control and db/db mice (Chen et al. (1996) Cell 84:491-495) was analyzed. Frozen organs from 10 week old and 35-week old male db/db mice and control db/+ mice were obtained. RNA was isolated with TRIzol reagent (Invitrogen) and QIAGEN RNeasy (Qiagen) according to the manufacturers' instructions. Total RNA (1 μg) was reverse transcribed according to the manufacturer's instructions (iScript cDNA synthesis kit, Bio-Rad). qPCR was performed using Platinum SYBR green SuperMix (Invitrogen) and 25 ng cDNA per reaction. Expression levels were normalized to the expression of L19.

Primer sequences were:

| | |
|---|---|
| mNdufa5 | |
| Fwd-ATCACCTTCGAGAAGCTGGA, | (SEQ ID NO: 104) |
| Rew-ACTTCACCACCCTGAAGCAA; | (SEQ ID NO: 105) |
| mCycs | |
| Fwd-CCAAATCTCCACGGTCTGTT, | (SEQ ID NO: 106) |
| Rew-CCAGGTGATGCCTTTGTTCT; | (SEQ ID NO: 107) |
| mVegfb | |
| Fwd-TCTGAGCATGGAACTCATGG, | (SEQ ID NO: 108) |
| Rew-TCTGCATTCACATTGGCTGT; | (SEQ ID NO: 109) |
| mFatp3 | |
| Fwd-CGCAGGCTCTGAACCTGG, | (SEQ ID NO: 110) |
| Rew-TCGAAGGTCTCCAGACAGGAG. | (SEQ ID NO: 111) |

As shown in FIG. 26, diabetic mice, like diabetic patients, have lower expression of mitochondrial genes (markers Ndufa5 and Cycs). The expression of Vegfb and its downstream target gene Fatp3 is however normal in diabetic mice, suggesting intact capacity for lipid uptake and reduced capacity for lipid oxidation.

The acute effects of anti-VEGF-B treatment were assessed by administering anti-VEGF-B mAbs to db/db animals and determining blood glucose and insulin parameters. Twenty mice were evaluated, with five males and five females in each group. The 2H10 group received the 2H10 anti-VEGF-B antibody (CSL Limited, Victoria, Australia, batch 20080307; U.S. Patent Publication No. 2008/0260729; Leonard et al. Journal of Molecular Biology 384:1203-1217). 2H10 is a neutralizing anti-VEGF-B antibody that inhibits binding of VEGF-B to VEGFR1. Control group received isotype matched C44 mouse antibody.

Treatment was started when mice were 7 weeks old (pre-diabetic) and continued for 6 weeks, with injection (400 ug antibody i.p. per mouse per injection, dissolved in 200 ul sterile PBS) and weighing 2 times/week. Blood glucose from the tail vein was measured once a week after 2 hours of food deprivation using a Bayer Ascenia Counter Blood Glucose Meter and glucose strips. After 6 weeks the mice were starved 18 hrs and intraperitoneal glucose tolerance testing (IPGTT) was performed.

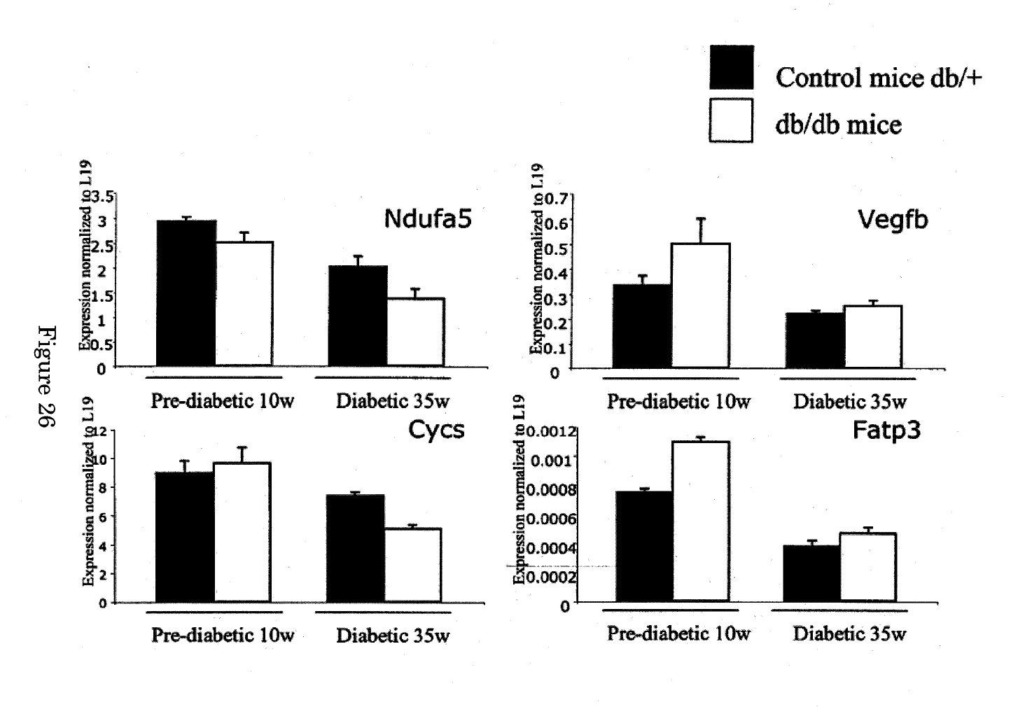

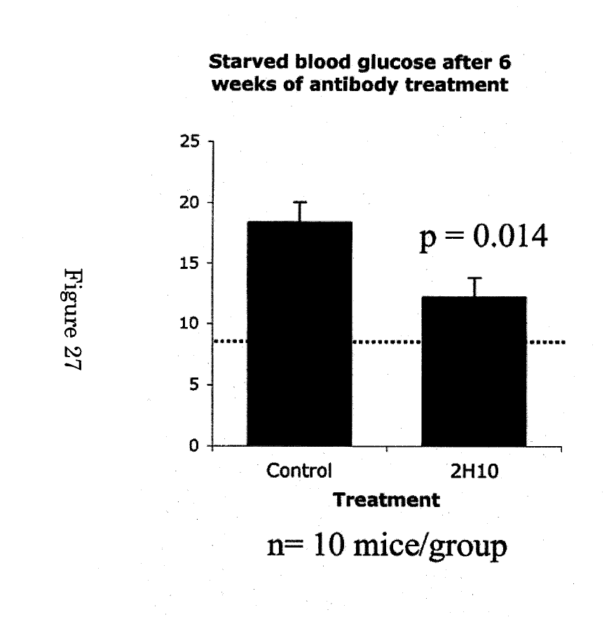

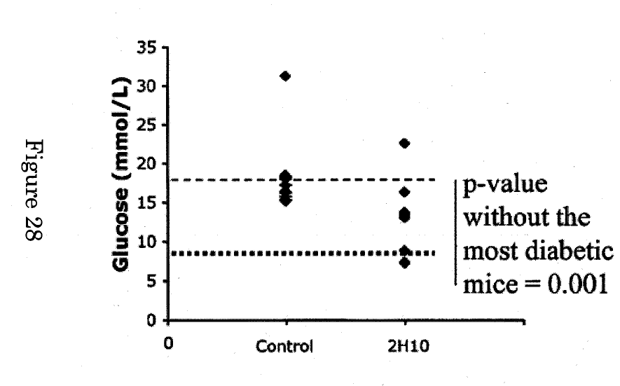

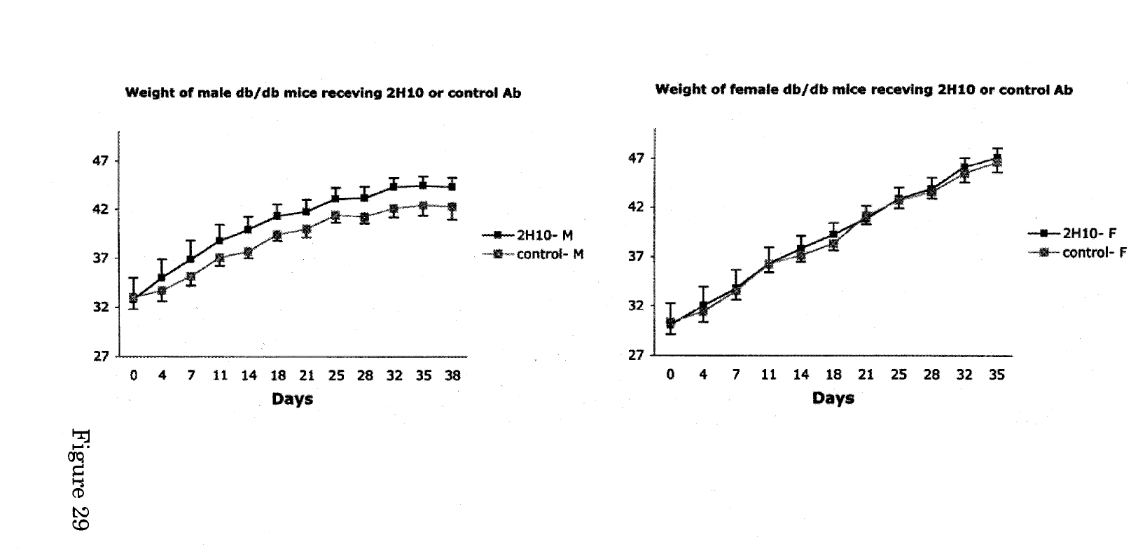

FIGS. 27 and 28 show fasting blood glucose (mmol/L) after six weeks of antibody treatment. FIG. 29 shows body weight curves, and shows that males given 2H10 gained 5% more weight (non-significant change) than control-treated males.

Plasma analysis was conducted as follows. Glucose was measured from the tail vein of normally fed or over-night fasted age-matched 8-12 week old male mice using a Contour Blood Glucose Meter (Ascensia). For all the other analysis, blood was collected from hearts of Avertin-anesthetized mice using EDTA syringes, and centrifuged for 1 min at 16,000 g. TG and NEFA content in plasma was determined using the Serum Triglyceride Determination Kit (Sigma), and the Wako NEFA C test kit (Wako Diagnostics), respectively, according to the manufactures' instructions.

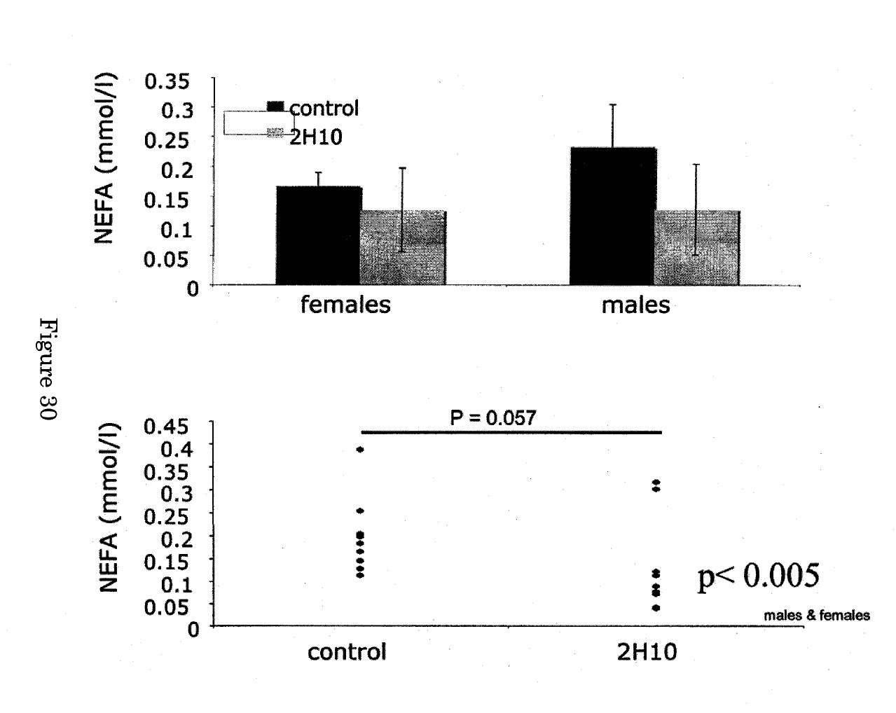

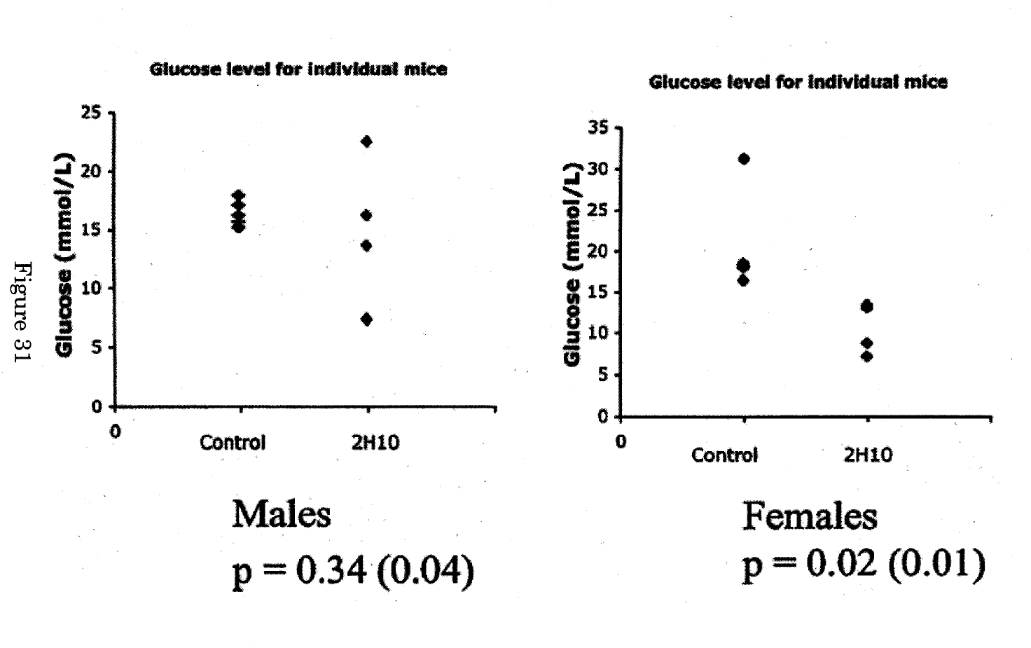

FIG. 30 shows non-esterified fatty acids (NEFA) in plasma in db/db mice treated with 2H10 or control C44 antibody. FIG. 31 shows glucose levels for individual mice, and demonstrates that females respond better to anti-VEGF-B treatment.

To determine the developmental/long term effects of VEGF-B inhibition, db/db mice were crossed onto a VEGF-B^{-/-} background. The characteristics of this phenotype include increased weight gain in male but not female mice, lower plasma levels of free fatty acids after starvation, all other plasma levels tested normal, and lower expression of FATPs and less ORO staining (neutral lipids) in heart, muscle, and liver tissue.

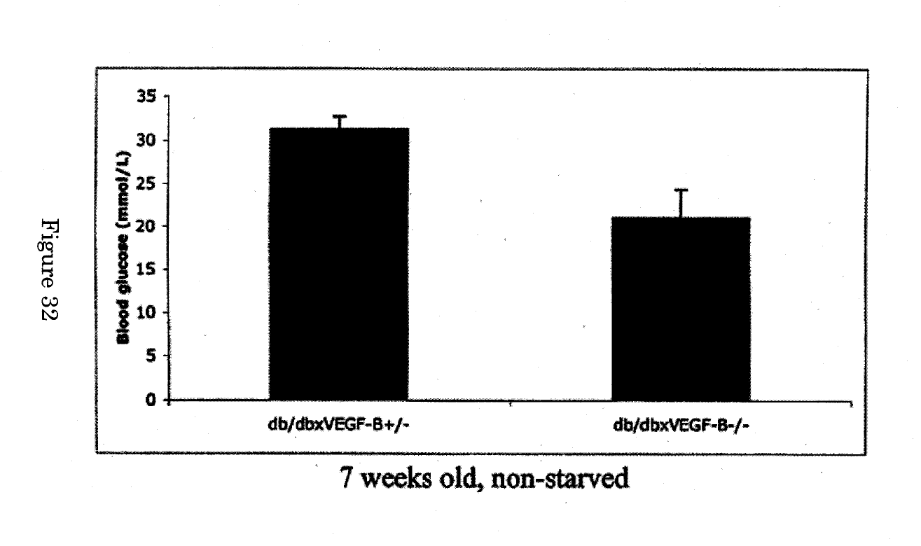

FIG. 32 shows blood glucose levels in VEGF-B^{-/-} mice on a db/db background and littermate controls (2 mice and 3 littermate controls). Db/db mice on a VEGF-B^{-/-} background have lower basal plasma glucose.

1. A method of reducing lipid accumulation in a mammalian subject, comprising:
administering to a mammalian subject in need of treatment to reduce lipid accumulation a composition that comprises a VEGF-B inhibitor, in an amount effective to reduce lipid accumulation in a tissue in the subject.

2. The method according to claim 1, further comprising monitoring lipid markers in a biological sample comprising a tissue or fluid from the subject.

3. The method of claim 1 wherein the VEGF-B inhibitor is an anti-VEGF-B antibody.

4. A method of stimulating glucose metabolism in a mammalian subject, comprising administering to a mammalian subject in need of treatment for a condition characterized by elevated blood glucose a composition that comprises a VEGF-B inhibitor, in an amount effective to stimulate glucose metabolism in the subject.

5. The method according to claim 4, further comprising monitoring glucose levels in the blood of the subject.

6. The method of any one of claim 4 wherein the VEGF-B inhibitor is an anti-VEGF-B antibody.

7. The method according to claim 4, comprising selecting for treatment a subject with at least one disease or condition selected from the group consisting of: obesity, insulin resistance, diabetes, hepatic steatosis, cardiovascular disease and metabolic syndrome.

8. The method according to claim 1, wherein the tissue is selected from the group consisting of the liver, kidney, skeletal muscle, and combinations thereof.

9. The method according to claim 1, wherein the subject has type II diabetes.

10. The method according to claim 1, wherein the VEGF-B inhibitor is selected from the group consisting of:
(a) an anti-VEGF-B₁₆₇ antibody;
(b) an anti-VEGF-B₁₈₆ antibody;
(c) an anti-VEGFR-1 antibody;
(d) a polypeptide that comprises an antigen binding domain of (a), (b) or (c) and that binds said antigen;
(e) an antisense oligonucleotide that inhibits VEGF-B transcription or translation;
(f) an aptamer that inhibits VEGF-B₁₆₇ and VEGF-B₁₈₆;
(g) an siRNA that inhibits VEGF-B translation;
(h) small molecule inhibitor to VEGF-R1, and
(i) combinations thereof.

11. (canceled)

12. (canceled)

13. (canceled)

14. (canceled)

15. (canceled)

16. (canceled)

17. A method of inhibiting expression of fatty acid transport proteins (FATPs) in a mammalian subject comprising administering to said subject a composition comprising an anti-VEGF-B antibody in an amount effective to inhibiting expression of FATPs in said subject.

18. The method according to claim 17 wherein said FATPs are selected from the group consisting of FATP1, FATP3, FATP4, FATP6, LPL, and combinations thereof.

19. The method according to claim 17 wherein said FATP is selected from the group consisting of FATP3, FATP4 and combinations thereof.

20. The method according to claim 17 wherein said FATPs are FATP3 and FATP4.

21. (canceled)

22. The method according to claim 4 wherein the subject has type II diabetes.

23. The method according to claim 4 wherein the VEGF-B inhibitor is selected from the group consisting of:
(a) an anti-VEGF-B₁₆₇ antibody;
(b) an anti-VEGF-B₁₈₆ antibody;
(c) an anti-VEGFR-1 antibody;
(d) a polypeptide that comprises an antigen binding domain of (a), (b) or (c) and that binds said antigen;
(e) an antisense oligonucleotide that inhibits VEGF-B transcription or translation;
(f) an aptamer that inhibits VEGF-B₁₆₇ and VEGF-B₁₈₆;
(g) an siRNA that inhibits VEGF-B translation;
(h) small molecule inhibitor to VEGF-R1, and
(i) combinations thereof.
